SEQUENCE LISTING (103 sequences)

SEQ ID NO: 1 - protein, 13 aa, Artificial Sequence
  Synthetic
LVILERHRQTRSS

SEQ ID NO: 2 - protein, 12 aa, Artificial Sequence
  Synthetic
LERHRQTRSSTE

SEQ ID NO: 3 - protein, 15 aa, Artificial Sequence
  Synthetic
RHRQTRSSTETFLFH

SEQ ID NO: 4 - protein, 11 aa, Artificial Sequence
  Synthetic
RHRQTRSSTET

SEQ ID NO: 5 - protein, 11 aa, Artificial Sequence
  Synthetic
RQTRSSTETFL

SEQ ID NO: 6 - protein, 10 aa, Artificial Sequence
  Synthetic
RQTRSSTETF

SEQ ID NO: 7 - protein, 11 aa, Artificial Sequence
  Synthetic
ERHRQTRSSTE

SEQ ID NO: 8 - protein, 10 aa, Artificial Sequence
  Synthetic
RHRQTRSSTE

SEQ ID NO: 9 - protein, 11 aa, Artificial Sequence
  Synthetic
RHRQTRSSTET

SEQ ID NO: 10 - protein, 15 aa, Artificial Sequence
  Synthetic
LERHRQTRSSTETFL

SEQ ID NO: 11 - protein, 8 aa, Artificial Sequence
  Synthetic
RQTRSSTE

SEQ ID NO: 12 - protein, 9 aa, Artificial Sequence
  Synthetic
RQTRSSTET

SEQ ID NO: 13 - protein, 9 aa, Artificial Sequence
  Synthetic
HRQTRSSTE

SEQ ID NO: 14 - protein, 21 aa, Artificial Sequence
  Synthetic
LAIVHAVHAYRHRRLLSIHIT

SEQ ID NO: 15 - protein, 17 aa, Artificial Sequence
  Synthetic
AIVHAVHAYRHRRLLSI

SEQ ID NO: 16 - protein, 12 aa, Artificial Sequence
  Synthetic
IVHAVHAYRHRR

SEQ ID NO: 17 - protein, 11 aa, Artificial Sequence
  Synthetic
VHAVHAYRHRR

SEQ ID NO: 18 - protein, 10 aa, Artificial Sequence
  Synthetic
HAVHAYRHRR

SEQ ID NO: 19 - protein, 14 aa, Artificial Sequence
  Synthetic
HAVHAYRHRRLLSI

SEQ ID NO: 20 - protein, 13 aa, Artificial Sequence
  Synthetic
HAVHAYRHRRLLS

SEQ ID NO: 21 - protein, 12 aa, Artificial Sequence
  Synthetic
HAVHAYRHRRLL

SEQ ID NO: 22 - protein, 11 aa, Artificial Sequence
  Synthetic
HAVHAYRHRRL

SEQ ID NO: 23 - protein, 10 aa, Artificial Sequence
  Synthetic
YRHRRLLSIH

SEQ ID NO: 24 - protein, 11 aa, Artificial Sequence
  Synthetic
RHRRLLSIHIT

SEQ ID NO: 25 - protein, 9 aa, Artificial Sequence
  Synthetic
RHRRLLSIH

SEQ ID NO: 26 - protein, 11 aa, Artificial Sequence
  Synthetic
HAYRHRRLLSI

SEQ ID NO: 27 - protein, 12 aa, Artificial Sequence
  Synthetic
VHAYRHRRLLSI

SEQ ID NO: 28 - protein, 13 aa, Artificial Sequence
  Synthetic
AVHAYRHRRLLSI

SEQ ID NO: 29 - protein, 14 aa, Artificial Sequence
  Synthetic
AAVHAYRHRRLLSI

SEQ ID NO: 30 - protein, 15 aa, Artificial Sequence
  Synthetic
HAAVHAYRHRRLLSI

SEQ ID NO: 31 - protein, 16 aa, Artificial Sequence
  Synthetic
IVHAVHAYRHRRLLSI

SEQ ID NO: 32 - protein, 15 aa, Artificial Sequence
  Synthetic
VHAVHAYRHRRLLSI

SEQ ID NO: 33 - protein, 14 aa, Artificial Sequence
  Synthetic
XAVHAYRHRRLLSI

SEQ ID NO: 34 - protein, 14 aa, Artificial Sequence
  Synthetic
HXVHAYRHRRLLSI

SEQ ID NO: 35 - protein, 13 aa, Artificial Sequence
  Synthetic
HAXAYRHRRLLSI

SEQ ID NO: 36 - protein, 14 aa, Artificial Sequence
  Synthetic
HAXHAYRHRRLLSI

SEQ ID NO: 37 - protein, 14 aa, Artificial Sequence
  Synthetic
HAVAAYRHRRLLSI

SEQ ID NO: 38 - protein, 14 aa, Artificial Sequence
  Synthetic
HAVHAARHRRLLSI

SEQ ID NO: 39 - protein, 14 aa, Artificial Sequence
  Synthetic
HAVHAYAHRRLLSI

SEQ ID NO: 40 - protein, 14 aa, Artificial Sequence
  Synthetic
HAVHAYRARRLLSI

SEQ ID NO: 41 - protein, 14 aa, Artificial Sequence
  Synthetic
HAVHAYRHARLLSI

SEQ ID NO: 42 - protein, 14 aa, Artificial Sequence
  Synthetic
HAVHAYRHRALLSI

SEQ ID NO: 43 - protein, 14 aa, Artificial Sequence
  Synthetic
HAVHAYRHRRALSI

SEQ ID NO: 44 - protein, 14 aa, Artificial Sequence
  Synthetic
HAVHAYRHRRLASI

SEQ ID NO: 45 - protein, 14 aa, Artificial Sequence
  Synthetic
HAVHAYRHRRLLAI

SEQ ID NO: 46 - protein, 14 aa, Artificial Sequence
  Synthetic
HAVHAYRHRRLLSA

SEQ ID NO: 47 - protein, 14 aa, Artificial Sequence
  Synthetic
HAVXAYRHRRLLSI

SEQ ID NO: 48 - protein, 14 aa, Artificial Sequence
  Synthetic
HAVHXYRHRRLLSI

SEQ ID NO: 49 - protein, 14 aa, Artificial Sequence
  Synthetic
HAVHAXRHRRLLSI

SEQ ID NO: 50 - protein, 14 aa, Artificial Sequence
  Synthetic
HAVHAYXHRRLLSI

SEQ ID NO: 51 - protein, 14 aa, Artificial Sequence
  Synthetic
HAVHAYRXRRLLSI

SEQ ID NO: 52 - protein, 14 aa, Artificial Sequence
  Synthetic
HAVHAYRHXRLLSI

SEQ ID NO: 53 - protein, 14 aa, Artificial Sequence
  Synthetic
HAVHAYRHRXLLSI

SEQ ID NO: 54 - protein, 14 aa, Artificial Sequence
  Synthetic
HAVHAYRHRRXLSI

SEQ ID NO: 55 - protein, 14 aa, Artificial Sequence
  Synthetic
HAVHAYRHRRLXSI

SEQ ID NO: 56 - protein, 14 aa, Artificial Sequence
  Synthetic
HAVHAYRHRRLLXI

SEQ ID NO: 57 - protein, 14 aa, Artificial Sequence
  Synthetic
HAVHAYRHRRLLFI

SEQ ID NO: 58 - protein, 14 aa, Artificial Sequence
  Synthetic
HAVHAYRHRRLLVI

SEQ ID NO: 59 - protein, 13 aa, Artificial Sequence
  Synthetic
HAVHAYRRRRLLS

SEQ ID NO: 60 - protein, 14 aa, Artificial Sequence
  Synthetic
HAVHAYRRRRLLSI

SEQ ID NO: 61 - protein, 21 aa, Artificial Sequence
  Synthetic
VVHRLRQAQRRPQRQKAVRVA

SEQ ID NO: 62 - protein, 11 aa, Artificial Sequence
  Synthetic
RQAQRRPQRQK

SEQ ID NO: 63 - protein, 17 aa, Artificial Sequence
  Synthetic
RQAQRRPQRQKAVRVAI

SEQ ID NO: 64 - protein, 13 aa, Artificial Sequence
  Synthetic
RLRQAQRRPQRQK

SEQ ID NO: 65 - protein, 13 aa, Artificial Sequence
  Synthetic
RQAQRRPQRQKAV

SEQ ID NO: 66 - protein, 17 aa, Artificial Sequence
  Synthetic
GVVHRLRQAQRRPQRQK

SEQ ID NO: 67 - protein, 23 aa, Artificial Sequence
  Synthetic
GVVHRLRQAQRRPQRQKAVRVAI

SEQ ID NO: 68 - protein, 15 aa, Artificial Sequence
  Synthetic
VHRLRQAQRRPQRQK

SEQ ID NO: 69 - protein, 16 aa, Artificial Sequence
  Synthetic
RLRQAQRRPQRQKAVR

SEQ ID NO: 70 - protein, 11 aa, Artificial Sequence
  Synthetic
RLRQAQRRPQR

SEQ ID NO: 71 - protein, 15 aa, Artificial Sequence
  Synthetic
RLRQAQRRPQRQKAV

SEQ ID NO: 72 - protein, 15 aa, Artificial Sequence
  Synthetic
RQAQRRPQRQKAVRV

SEQ ID NO: 73 - protein, 10 aa, Artificial Sequence
  Synthetic
RRPQRQKAVR

SEQ ID NO: 74 - protein, 16 aa, Artificial Sequence
  Synthetic
HFRKERIEGLRKRRRL

SEQ ID NO: 75 - protein, 12 aa, Artificial Sequence
  Synthetic
RPQRQKAVRVAI

SEQ ID NO: 76 - protein, 14 aa, Artificial Sequence
  Synthetic
RQAQRRPQRQKAVR

SEQ ID NO: 77 - protein, 15 aa, Artificial Sequence
  Synthetic
RQAQRRPQRQKAVRA

SEQ ID NO: 78 - protein, 17 aa, Artificial Sequence
  Synthetic
RLRNANRRPNRNKAVRV

SEQ ID NO: 79 - protein, 11 aa, Artificial Sequence
  Synthetic
RLRQAQRRPQR

SEQ ID NO: 80 - protein, 12 aa, Artificial Sequence
  Synthetic
RLRQAQRRPQRQ

SEQ ID NO: 81 - protein, 14 aa, Artificial Sequence
  Synthetic
RLRQAQRRPQRQKA

SEQ ID NO: 82 - protein, 17 aa, Artificial Sequence
  Synthetic
RLRQAQRRPQRQKAVRV

SEQ ID NO: 83 - protein, 17 aa, Artificial Sequence
  Synthetic
RLRTATRRPTRTKAVRV

SEQ ID NO: 84 - protein, 14 aa, Artificial Sequence
  Synthetic
GRLRQAQRRPQRQK

SEQ ID NO: 85 - protein, 12 aa, Artificial Sequence
  Synthetic
HRLRQAQRRPQR

SEQ ID NO: 86 - protein, 14 aa, Artificial Sequence
  Synthetic
HRLRQAQRRPQRQK

SEQ ID NO: 87 - protein, 20 aa, Artificial Sequence
  Synthetic
GSGRLRQAQRRPQRQKAVRV

SEQ ID NO: 88 - protein, 17 aa, Artificial Sequence
  Synthetic
AGVKFRSDLSRLLTKLG

SEQ ID NO: 89 - protein, 13 aa, Artificial Sequence
  Synthetic
RSDLSRLLTKLGS

SEQ ID NO: 90 - protein, 10 aa, Artificial Sequence
  Synthetic
RSDLSRLLTK

SEQ ID NO: 91 - protein, 13 aa, Artificial Sequence
  Synthetic
LLTKLGSTGPASL

SEQ ID NO: 92 - protein, 13 aa, Artificial Sequence
  Synthetic
LSRLLTKLGSTGP

SEQ ID NO: 93 - protein, 23 aa, Artificial Sequence
  Synthetic
VKFRSDLSRLLTKLGSTGPASLS

SEQ ID NO: 94 - protein, 20 aa, Homo Sapiens
LVILERHRQTRSSTETFLFH

SEQ ID NO: 95 - protein, 21 aa, Homo Sapiens
LAIVHAVHAYRHRRLLSIHIT

SEQ ID NO: 96 - protein, 21 aa, Homo Sapiens
VVHRLRQAQRRPQRQKAVRVA

SEQ ID NO: 97 - protein, 48 aa, Homo Sapiens
AGVKFRSDLSRLLTKLGCTGPASLCQLFPSWRRSSLSESENATSLTTF

SEQ ID NO: 98 - protein, 14 aa, Artificial Sequence
  Synthetic
HAAHAYRHRRLLSI

SEQ ID NO: 99 - protein, 14 aa, Artificial Sequence
  Synthetic
HAHXAYRHRRLLSI

SEQ ID NO: 100 - protein, 13 aa, Artificial Sequence
  Synthetic
HAXHAYRHRRLLS

SEQ ID NO: 101 - protein, 13 aa, Artificial Sequence
  Synthetic
RRPQRQKAVRVAI

SEQ ID NO: 102 - protein, 16 aa, Artificial Sequence
  Synthetic
RQAQRRPQRQKAVRVA

SEQ ID NO: 103 - protein, 16 aa, Artificial Sequence
  Synthetic
RLRQAQRRPQRQKAVR

(19) United States
(12) Patent Application Publication
(10) Pub. No.: US 2011/0300167 A1
(43) Pub. Date: Dec. 8, 2011

(54) CXCR5 RECEPTOR COMPOUNDS

(76) Inventors: Thomas J. McMurry, Winchester, MA (US); Athan Kuliopulos, Winchester, MA (US); Lidija Covic, Boston, MA (US)

(21) Appl. No.: 13/127,666

(22) PCT Filed: Nov. 4, 2009
(86) PCT No.: PCT/US09/05976

§ 371 Date: Aug. 12, 2011

(30) Foreign Application Priority Data
Nov. 4, 2008 (US) 61/198297

(51) Int. Cl. A61P 37/00; C07K 7/08; C07K 7/06; A61P 35/02; A61P 35/04; A61K 38/17; A61P 37/02; A61P 19/04; A61P 31/18; A61P 29/00; A61K 38/10; A61K 38/08; C07K 14/435; A61P 25/00

(52) U.S. Cl. 424/185.1; 530/326; 530/327; 530/324; 530/329; 530/328; 530/325; 514/19.6; 514/19.8; 514/17.9; 514/3.8; 514/16.6

(57) ABSTRACT

The invention relates generally to compounds which are allosteric modulators (e.g., negative and positive allosteric modulators, allosteric agonists, and ago-allosteric modulators) of the G protein coupled receptor CXCR5. The CXCR5 receptor compounds are derived from the intracellular loops and domains of the CXCR5 receptor. The invention also relates to the use of these CXCR5 receptor compounds and pharmaceutical compositions comprising the CXCR5 receptor compounds in the treatment of diseases and conditions associated with CXCR5 receptor modulation such as autoimmune diseases including lupus, HIV and rheumatoid arthritis, Primary Sjogren's Syndrome, chronic lymphocytic leukemia, Burkitt Lymphoma, colon and breast cancer tumor metastasis, Multiple Sclerosis and compromised immune function.

### RELATED APPLICATION

This application claims the benefit of U.S. Provisional Application No. 61/198,297, filed on Nov. 4, 2008. The entire teachings of the above application are incorporated herein by reference.

### BACKGROUND OF THE INVENTION

G protein coupled receptors (GPCRs) constitute one of the largest families of genes in the human genome. GPCRs are integral membrane signaling proteins. Hydrophobicity mapping of the amino acid sequences of G-protein coupled receptors has led to a model of the typical G-protein-coupled receptor as containing seven hydrophobic membrane-spanning regions with the amino terminal on the extracellular side of the membrane and the carboxyl terminal on the intracellular side of the membrane.

GPCRs mediate the transmission of intracellular signals (“signal transduction”) by activating guanine nucleotide-binding proteins (G proteins) to which the receptor is coupled. GPCRs are activated by a wide range of endogenous stimuli, including peptides, amino acids. hormones, light, and metal ions. The following reviews are incorporated by reference: Hill, British J. Pharm 147: s27 (2006); Palczeski, Ann. Rev. Biochemistry 75: 743-767 (2006); Dorsham & Gutkind, Nature Reviews 7: 79-94 (2007); Kobilka & Schertler, Trends Pharmacol Sci. 2: 79-83 (2008).

GPCRs are important targets for drug discovery as they are involved in a wide range of cellular signaling pathways and are implicated in many pathological conditions (e.g., cardiovascular and mental disorders, cancer, AIDS). In fact, GPCRs are targeted by 40-50% of approved drugs, illustrating the critical importance of this class of pharmaceutical targets. Interestingly, this number represents only about 30 GPCRs, a small fraction of the total number of GPCRs thought to be relevant to human disease. Over 1000 GPCRs are known in the human genome, and GPCRs remain challenging targets from a research and development perspective in part because these a membrane bound receptors with complex pharmacology.

There remains a need for the development of new pharmaceuticals that are GPCR modulators (e.g., agonists, partial agonists, inverse agonists and antagonists) and especially those that are allosteric modulators of GPCRs (e.g., negative and positive allosteric modulators, allosteric agonists, and ago-allosteric modulators).

### SUMMARY OF THE INVENTION

The invention relates generally to compounds which are allosteric modulators (e.g., negative and positive allosteric modulators, allosteric agonists, and ago-allosteric modulators) of the G protein coupled receptor CXCR5. The CXCR5 receptor compounds are derived from the intracellular loops and domains of the CXCR5 receptor. The invention also relates to the use of these CXCR5 receptor compounds and pharmaceutical compositions comprising the CXCR5 receptor compounds in the treatment of diseases and conditions associated with CXCR5 receptor modulation such as autoimmune diseases including lupus, HIV and rheumatoid arthritis, Primary Sjögren's Syndrome, chronic lymphocytic leukemia, Burkitt Lymphoma, colon and breast cancer tumor metastasis, Multiple Sclerosis and compromised immune function.

More specifically, the invention relates to compounds represented by Formula I:

TLP,

or pharmaceutically acceptable salts thereof, wherein:
P is a peptide comprising at least three contiguous amino-acid residues
of an intracellular i1, i2, i3 loop or an intracellular i4 domain of the CXCR5 receptor;
L is a linking moiety represented by C(O) and bonded to P at an N terminal nitrogen of an N-terminal amino-acid residue;
and T is a lipophilic tether moiety bonded to L.

The invention also relates to pharmaceutical compositions comprising one or more compounds of the invention and a carrier, and the use of the disclosed compounds and compositions in methods of treating diseases and conditions responsive to modulation (inhibition or activation) of the CXCR5 receptor.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1A-1D are a series of graphical representations of compounds of the invention derived from the i1 loop in a chemotaxis assay as compared with vehicle.

FIGS. 2A-2D are a series of graphical representations of compounds of the invention derived from the i2 loop in a chemotaxis assay as compared with vehicle.

FIGS. 3A-3E are a series of graphical representations of compounds of the invention derived from the i3 loop in a chemotaxis assay as compared with vehicle.

FIGS. 4A-4B are a series of graphical representations of compounds of the invention derived from the i4 domain in a chemotaxis assay as compared with vehicle.

FIG. 5 is a graphical representation of a cAMP assay that was performed in the presence of CXCR5 Compound 14 of the invention.

FIG. 6 is a graphical representation of β-arrestin data activity of CXCR5 Compound 14 of the invention.

### DETAILED DESCRIPTION OF THE INVENTION

A description of example embodiments of the invention follows.

### G Protein Coupled Receptors (GPCRs)

G protein coupled receptors (GPCRs) constitute one of the largest superfamilies of genes in the human genome;'these transmembrane proteins enable the cell the respond to its environment by sensing extracellular stimuli and initiating intracellular signal transduction cascades. GPCRs mediate signal transduction through the binding and activation of guanine nucleotide-binding proteins (G proteins) to which the receptor is coupled. Wide arrays of ligands bind to these receptors, which in turn orchestrate signaling networks integral to many cellular functions. Diverse GPCR ligands include small proteins, peptides, amino acids, biogenic amines, lipids, ions, odorants and even photons of light. The following reviews are incorporated by reference: Hill, British J. Pharm 147: s27 (2006); Dorsham & Gutkind, Nature Reviews 7: 79-94 (2007).

In addition to modulating a diverse array of homeostatic processes, GPCR signaling pathways are integral components of many pathological conditions (e.g., cardiovascular and mental disorders, cancer, AIDS). In fact, GPCRs are targeted by 40-50% of approved drugs illustrating the critical importance of this class of pharmaceutical targets. Interestingly, this number represents only about 30 GPCRs, a small fraction of the total number of GPCRs thought to be relevant to human disease. GPCRs are membrane bound receptors that exhibit complex pharmacological properties and remain challenging targets from a research and development perspective. Given their importance in human health combined with their prevalence (over 1000 known GPCRs in the human genome) GPCRs represent an important target receptor class for drug discovery and design.

GPCRs are integral membrane proteins that mediate diverse signaling cascades through an evolutionarily conserved structural motif. All GPCRs are thought to consist of seven hydrophobic transmembrane spanning α-helices with the amino terminus on the extracellular side of the membrane and the carboxyl terminus on the intracellular side of the membrane. The transmembrane helices are linked together sequentially by extracellular (e1, e2, e3) and intracellular (cytoplasmic) loops (i1, i2, i3). The intracellular loops or domains are intimately involved in the coupling and turnover of G proteins and include: il, which connects TM1-TM2; i2, connecting TM3-TM4; i3, connecting TM5-TM6; and a portion of the C-terminal cytoplasmic tail (domain 4). Due in part to the topological homology of the 7TM domains and the recent high resolution crystal structures of several GPCRs (Palczewski et al., Science 289, 739-45 (2000), Rasmussen, S. G. et al., Nature 450, 383-7 (2007)) skilled modelers are now able to predict the general boundaries of GPCR loop domains through the alignment of several related receptors. These predictions are aided in part by a number of programs used by computational biologists, including EMBOSS, ClustalW2, Kalign, and MAFFT (Multiple Alignment using Fast Fourier Transform). Importantly, many of these programs are publically available (see, for example, The European Bioinformatics Institute (EMBL-EBI) web site http://www.ebi.ac.uk/Tools/) and most have web-based interfaces.

GPCR mediated signal transduction is initiated by the binding of a ligand to its cognate receptor. In many instances GPCR ligand binding is believed to take place in a hydrophilic pocket generated by a cluster of helices near the extracellular domain. However, other ligands, such as large peptides, are thought to bind to the extracellular region of protein and hydrophobic ligands are postulated to intercalate into a receptor binding pocket through the membrane between gaps in the helices. The process of ligand binding induces conformational changes within the receptor. These changes involve the outward movement of helix 6, which in turn alters the conformations of the intracellular loops and ultimately results in a receptor form that is able to bind and activate a heterotrimeric G protein (Farrens, D., et al. Science 274, 768-770 (1996), Gether, U. and Kobilka, B., J. Biol. Chem. 273, 17979-17982 (1998)). Upon binding the receptor catalyzes the exchange of GTP for GDP in the alpha subunit of the heterotrimeric G protein, which results in a separation of the G protein from the receptor as well a dissociation of the alpha and beta/gamma subunits of the G protein itself Notably, this process is catalytic and results in signal amplification in that activation of one receptor may elicit the activation and turnover of numerous G proteins, which in turn may regulate multiple second messenger systems. Signaling diversity is further achieved through the existence of numerous G protein types as well as differing isoforms of alpha, beta and gamma subunits. Typically, GPCRs interact with G proteins to regulate the synthesis or inhibition of intracellular second messengers such as cyclic AMP, inositol phosphates, diacylglycerol and calcium ions, thereby triggering a cascade of intracellular events that eventually leads to a biological response.

GPCR signaling may be modulated and attenuated through cellular machinery as well as pharmacological intervention. Signal transduction may be ‘switched off’ with relatively fast kinetics (seconds to minutes) by a process called rapid desensitization. For GPCRs, this is caused by a functional uncoupling of receptors from heterotrimeric G proteins, without a detectable change in the total number of receptors present in cells or tissues. This process involves the phosphorylation of the receptor C terminus, which enables the protein arrestin to bind to the receptor and occlude further G protein coupling. Once bound by arrestin the receptor may be internalized into the cell and either recycled back to the cell surface or degraded. The alpha subunit of the G protein possesses intrisic GTPase activity, which attenuates signaling and promotes re-association with the beta/gamma subunits and a return to the basal state. GPCR signaling may also be modulated pharmacologically. Agonist drugs act directly to activate the receptors, whereas antagonist drugs act indirectly to block receptor signaling by preventing agonist activity through their associating with the receptor. GPCR binding and signaling can also be modified through allosteric modulation, that is by ligands that bind not at the orthosteric binding site but through binding at an allosteric site elsewhere in the receptors. Allosteric modulators can include both positive and negative modulators of orthosteric ligand mediated activity, allosteric agonists (that act in the absence of the orthosteric ligand and ago-allosteric modulators (ligands that have agonist activity on their own but that can also modulate the activity of the orthosteric ligand).

The large superfamily of GPCRs may be divided into subclasses based on structural and functional similarities. GPCR families include Class A Rhodopsin like, Class B Secretin like, Class C Metabotropic glutamate/pheromone, Class D Fungal pheromone, Class E cAMP receptors (Dictyostelium), the Frizzled/Smoothened family, and various orphan GPCRs. In addition, putative families include Ocular albinism proteins, Insect odorant receptors, Plant Mlo receptors, Nematode chemoreceptors, Vomeronasal receptors (VIR & V3R) and taste receptors.

Class A GPCRs, also called family A or rhodopsin-like, are the largest class of receptors and characteristically have relatively small extracellular loops that form the basis for selectivity vs. endogenous agonists and small-molecule drugs. In addition, Class A receptors also have relatively small intracellular loops. Class A receptors include amine family members such as dopamine and serotonin, peptide members such as chemokine and opioid, the visual opsins, odorant receptors and an array of hormone receptors.

CXCR5 is a Class A receptor belonging to the chemokine subfamily and has been implicated in conditions such as autoimmune diseases including Lupus, HIV and rheumatoid arthritis, Primary Sjögren's Syndrome, chronic lymphocytic leukemia, Burkitt Lymphoma, colon and breast cancer tumor metastasis, Multiple Sclerosis, and compromised immune function.

### Peptides

As defined herein, P is a peptide comprising at least three contiguous amino-acid residues (e.g., at least 3, 4, 5, 6, 7, 8, 9, 10, 11, 12, 13, 14, 15, 16, or 17) of an intracellular i1, i2 or i3 loop or intracellular i4 domain of the CXCR5 receptor. It is understood that, the N-terminal nitrogen of the N-terminal amino acid residue of P to which the linking moiety C(O) is bonded can be one of the at least three contiguous amino acid residues or it can be an amino acid residue distinct from the at least three contiguous amino acid residues.

Intracellular i1 loop as used herein refers to the loop which connects TM1 to TM2 and the corresponding transmembrane junctional residues.

Intracellular i2 loop as used herein refers to the loop which connects TM3 to TM4 and the corresponding transmembrane junctional residues.

Intracellular i3 loop as used herein refers to the loop which connects TM5 to TM6 and the corresponding transmembrane junctional residues.

Intracellular i4 domain as used herein refers to the C-terminal cytoplasmic tail and the transmembrane junctional residue.

In a specific embodiment, P comprises at least three, at least four, at least five, at least six, at least seven, at least eight, at least nine, at least ten, at least eleven, at least twelve, at least thirteen, at least fourteen, at least fifteen, at least sixteen, or at least seventeen contiguous amino acid residues of the intracellular i2 or i3 loop or intracellular i4 domain of the CXCR5 receptor

In a more specific embodiment, the at least three contiguous amino acids of P (e.g., at least 3, 4, 5, 6, 7, 8, 9, 10, 11, 12, 13, 14, 15, 16, or 17) are derived from the intracellular i 1, i2 or i3 loop or intracellular i4 domain of the CXCR5 receptor, wherein the amino acid sequence of each loop and the i4 domain is as defined in Table 1.

TABLE 1

| Intra- | |
|---|---|
| cellular | |
| Loop | |
| or | |
| Domain | CXCR5 Receptor |
| i1 | LVILERHRQTRSSTETFLFH (SEQ ID NO: 94) |
| i2 | LAIVHAVHAYRHRRLLSIHIT (SEQ ID NO: 95) |
| i3 | VVHRLRQAQRRPQRQKAVRVA (SEQ ID NO: 96) |
| i4 | AGVKFRSDLSRLLTKLGCTGPASLCQLFPSWRRSSL |
| | SESENATSLTTF (SEQ ID NO: 97) |

It is understood that in addition to the amino acids shown in the sequences in Table 1, the intracellular loop for the i1 loop, i2 loop, i3 loop and i4 domain can also include the transmembrane junctional residues. For example, the i1 loop can include SEQ ID NO: 1 where one or more residues from the transmembrane junctional residues are included on either the C-terminus, the N-terminus or both.

In another embodiment, P comprises at least three, at least four, at least five, at least six, at least seven, at least eight, at least nine, at least ten, at least eleven, at least twelve, at least thirteen, at least fourteen, at least fifteen, at least sixteen, or at least seventeen, contiguous amino acid residues of the i1 intracellular loop of the CXCR5 receptor.

In an even more specific embodiment, P is selected from the group consisting of SEQ ID NOS:1-13 as listed in Table 2 below.

TABLE 2

| CXCR5 | | |
|---|---|---|
| i-Loop | Sequence | SEQ ID: |
| i1 | LVILERHRQTRSS | 1 |
| i1 | LERHRQTRSSTE | 2 |
| il | RHRQTRSSTETFLFH | 3 |
| i1 | RHRQTRSSTET | 4 |
| i1 | RQTRSSTETFL | 5 |
| i1 | RQTRSSTETF | 6 |
| i1 | ERHRQTRSSTE | 7 |
| i1 | RHRQTRSSTE | 8 |
| i1 | RHRQTRSSTET | 9 |
| i1 | LERHRQTRSSTETFL | 10 |
| i1 | RQTRSSTE | 11 |
| il | RQTRSSTET | 12 |
| il | HRQTRSSTE | 13 |

In another specific embodiment, the at least three contiguous amino acids of P (e.g., at least 3, 4, 5, 6, 7, 8, 9, 10, 11, 12, 13, 14, 15, 16, or 17) are derived from the i2 intracellular loop of the CXCR5 receptor.

In a more specific embodiment, P is selected from the group consisting of SEQ ID NOS: 14-60 as listed in Table 3 below.

TABLE 3

| CXCR5 | | |
|---|---|---|
| i-Loop | Sequence | SEQ ID: |
| i2 | LAIVHAVHAYRHRRLLSIHIT | 14 |
| i2 | AIVHAVHAYRHRRLLSI | 15 |
| i2 | IVHAVHAYRHRR | 16 |
| i2 | VHAVHAYRHRR | 17 |
| i2 | HAVHAYRHRR | 18 |
| i2 | HAVHAYRHRRLLSI | 19 |
| i2 | HAVHAYRHRRLLS | 20 |
| i2 | HAVHAYRHRRLL | 21 |
| i2 | HAVHAYRHRRL | 22 |
| i2 | YRHRRLLSIH | 23 |
| i2 | RHRRLLSIHIT | 24 |
| i2 | RHRRLLSIH | 25 |
| i2 | HAYRHRRLLSI | 26 |
| i2 | VHAYRHRRLLSI | 27 |
| i2 | AVHAYRHRRLLSI | 28 |
| i2 | AAVHAYRHRRLLSI | 29 |
| i2 | HAAVHAYRHRRLLSI | 30 |
| i2 | IVHAVHAYRHRRLLSI | 31 |
| i2 | VHAVHAYRHRRLLSI | 32 |
| i2 | hAVHAYRHRRLLSI | 33 |
| i2 | HaVHAYRHRRLLSI | 34 |
| i2 | HAhAYRHRRLLSI | 35 |
| i2 | HAvHAYRHRRLLSI | 36 |
| i2 | HAVAAYRHRRLLSI | 37 |
| i2 | HAVHAARHRRLLSI | 38 |
| i2 | HAVHAYAHRRLLSI | 39 |
| i2 | HAVHAYRARRLLSI | 40 |
| i2 | HAVHAYRHARLLSI | 41 |
| i2 | HAVHAYRHRALLSI | 42 |
| i2 | HAVHAYRHRRALSI | 43 |
| i2 | HAVHAYRHRRLASI | 44 |
| i2 | HAVHAYRHRRLLAI | 45 |
| i2 | HAVHAYRHRRLLSA | 46 |
| i2 | HAVhAYRHRRLLSI | 47 |
| i2 | HAVHaYRHRRLLSI | 48 |
| i2 | HAVHAyRHRRLLSI | 49 |
| i2 | HAVHAYrHRRLLSI | 50 |
| i2 | HAVHAYRhRRLLSI | 51 |
| i2 | HAVHAYRHrRLLSI | 52 |
| i2 | HAVHAYRHRrLLSI | 53 |
| i2 | HAVHAYRHRRILSI | 54 |
| i2 | HAVHAYRHRRLISI | 55 |
| i2 | HAVHAYRHRRLLsI | 56 |
| i2 | HAVHAYRHRRLLFI | 57 |
| i2 | HAVHAYRHRRLLVI | 58 |
| i2 | HAVHAYRRRRLLS | 59 |
| i2 | HAVHAYRRRRLLSI | 60 |

In yet another specific embodiment, P comprises at least three contiguous amino (e.g., at least 3, 4, 5, 6, 7, 8, 9, 10, 11, 12, 13, 14, 15, 16, or 17) of the i3 intracellular loop of the CXCR5 receptor.

In a more specific embodiment, P is selected from the group consisting of SEQ ID NOS:61-87 as listed in Table 4 below.

TABLE 4

| CXCR5 | | |
|---|---|---|
| i-Loop | Sequence | SEQ ID: |
| i3 | VVHRLRQAQRRPQRQKAVRVA | 61 |
| i3 | RQAQRRPQRQK | 62 |
| i3 | RQAQRRPQRQKAVRVAI | 63 |
| i3 | RLRQAQRRPQRQK | 64 |
| i3 | RQAQRRPQRQKAV | 65 |
| i3 | GVVHRLRQAQRRPQRQK | 66 |
| i3 | GVVHRLRQAQRRPQRQKAVRVAI | 67 |
| i3 | VHRLRQAQRRPQRQK | 68 |
| i3 | RLRQAQRRPQRQKAVR | 69 |
| i3 | RLRQAQRRPQR | 70 |
| i3 | RLRQAQRRPQRQKAV | 71 |
| i3 | RQAQRRPQRQKAVRV | 72 |
| i3 | RRPQRQKAVR | 73 |
| i3 | HFRKERIEGLRKRRRL | 74 |
| i3 | RPQRQKAVRVAI | 75 |
| i3 | RQAQRRPQRQKAVR | 76 |
| i3 | RQAQRRPQRQKAVRA | 77 |
| i3 | RLRNANRRPNRNKAVRV | 78 |
| i3 | RLRQAQRRPQR | 79 |
| i3 | RLRQAQRRPQRQ | 80 |
| i3 | RLRQAQRRPQRQKA | 81 |
| i3 | RLRQAQRRPQRQKAVRV | 82 |
| i3 | RLRTATRRPTRTKAVRV | 83 |
| i3 | GRLRQAQRRPQRQK | 84 |
| i3 | HRLRQAQRRPQR | 85 |
| i3 | HRLRQAQRRPQRQK | 86 |
| i3 | GSGRLRQAQRRPQRQKAVRV | 87 |

In further specific embodiment, P comprises at least three contiguous amino (e.g., at least 3, 4, 5, 6, 7, 8, 9, 10, 11, 12, 13, 14, 15, 16, or 17) of the i4 intracellular domain of the CXCR5 receptor.

In a more specific embodiment, P is selected from the group consisting of SEQ ID NOS:88-93 as listed in Table 5 below.

TABLE 5

| CXCR5 | | SEQ |
|---|---|---|
| i-Loop | Sequence | ID: |
| i4 | AGVKFRSDLSRLLTKLG | 88 |
| i4 | RSDLSRLLTKLGS | 89 |
| i4 | RSDLSRLLTK | 90 |
| i4 | LLTKLGSTGPASL | 91 |
| i4 | LSRLLTKLGSTGP | 92 |
| i4 | VKFRSDLSRLLTKLGSTGPASLS | 93 |

It is understood that the sequences presented in Tables 2-5 can be optionally functionalized at the C-terminus. Functionalized at the C-terminus means that the acid moiety present at the C-terminus is replaced by some other functional group. Suitable functional groups include —C(O)N(R₂)₂, —C(O)OR₃, or C(O)NHC(O)OR₂, where R₂ is hydrogen or an alkyl group, for example, a (C₁-C₁₀) alkyl group and R₃ is an alkyl group, for example, a (C₁-C₁₀) alkyl group.

It is understood that as long as P comprises the indicated number of contiguous amino acids residues from the CXCR5 intracellular loop (i1, i2 or i3) or domain (i4) from which it is derived, the remainder of the peptide, if present, can be selected from:

(a) any natural amino acid residue, unnatural amino acid residue or a combination thereof;

(b) a peptide sequence comprising natural amino acid residues, non-natural amino acid residues and combinations thereof;

(c) a peptide sequence according to (b) comprising one or more peptide backbone modifications;

(d) a peptide sequence according to (c) comprising one or more retro-inverso peptide linkages;

(e) a peptide sequence according to (c) wherein one or more peptide bonds are replaced by

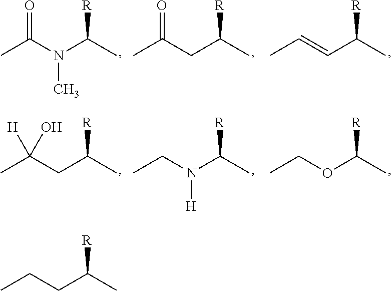

or a combination thereof;

(f) a peptide sequence according to (c) comprising one or more depsipeptide linkages, wherein the amide linkage is replaced with an ester linkage; and

(g) a peptide sequence according to (c) comprising one or more conformational restrictions; and

(h) a peptide sequence according to (c) comprising one or more of (d)-(g).

Furthermore, it is understood that even within the indicated number of contiguous amino acid residues derived from the GPCR intracellular loop (i1, i2 or i3) or domain (i4), there can be: peptide backbone modifications such as, but not limited to, those described in (e) above; retro-inverso peptide linkages; despsipeptide linkages; conformational restrictions; or a combination thereof.

It is noted that P of Formula I can be optionally functionalized at the C-terminus. Functionalized at the C-terminus means that the acid moiety present at the C-terminus is replaced by some other functional group. Suitable functional groups include —C(O)N(R₂)₂, —C(O)OR₃, or C(O)NHC(O)OR₂, where R₂ is hydrogen or an alkyl group, for example, a (C₁-C₁₀) alkyl group and R₃ is an alkyl group, for example, a (C₁-C₁₀) alkyl group. Functionalization of the C-terminus can result from the methods used to prepare.

Peptidomimetic as used herein refers to a compound comprising non-peptidic structural elements in place of a peptide sequence.

As used herein, the term “amino acid” includes both a naturally occurring amino acid and a non-natural amino acid.

As used herein, the term “naturally occurring amino acid” means a compound represented by the formula NH₂—CHR—COOH, wherein R is the side chain of a naturally occurring amino acids such as lysine, arginine, serine, tyrosine etc. as shown in the Table below.

Table of Common Naturally Occurring Amino Acids

| | Amino acid | Three letter code | One letter code |
|---|---|---|---|
| Non-polar; | alanine | Ala | A |
| neutral at | isoleucine | Ile | I |
| pH 7.4 | leucine | Leu | L |
| | methionine | Met | M |
| | phenylalanine | Phe | F |
| | proline | Pro | P |
| | tryptophan | Trp | W |
| | valine | Val | V |
| Polar, | asparagine | Asn | N |
| uncharged | cysteine | Cys | C |
| at pH 7.0 | glycine | Gly | G |
| | glutamine | Gln | Q |
| | serine | Ser | S |
| | threonine | Thr | T |
| | tyrosine | Tyr | Y |
| Polar; | glutamic acid | Glu | E |
| charged at | arginine | Arg | R |
| pH 7 | aspartic acid | Asp | D |
| | histidine | His | H |
| | lysine | Lys | K |

“Non-natural amino acid” means an amino acid for which there is no nucleic acid codon. Examples of non-natural amino acids include, for example, the D-isomers of the natural α-amino acids such as D-proline (D-P, D-Pro) as indicated above; natural α-amino acids with non-natural side chains (e.g.,

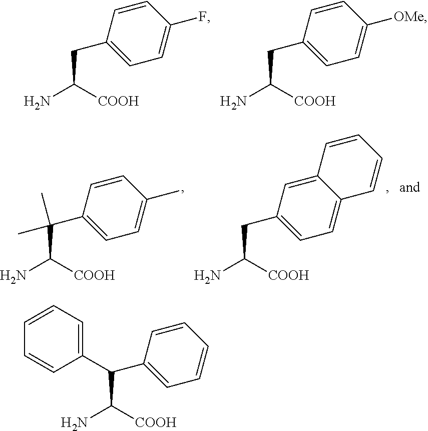

related to phenylalanine); Aib (aminobutyric acid), bAib (3-aminoisobutyric acid), Nva (norvaline), β-Ala, Aad (2-aminoadipic acid), bAad (3-aminoadipic acid), Abu (2-aminobutyric acid), Gaba (γ-aminobutyric acid), Acp (6-aminocaproic acid), Dbu (2,4-diaminobutryic acid), α-aminopimelic acid, TMSA (trimethylsilyl-Ala), alle (allo-isoleucine), Nle (norleucine), tert-Leu, Cit (citrulline), Orn (ornithine, O), Dpm (2,2′-diaminopimelic acid), Dpr (2,3-diaminopropionic acid), α or β-Nal, Cha (cyclohexyl-Ala), hydroxyproline, Sar (sarcosine), and the like.

Unnatural amino acids also include cyclic amino acids; and amino acid analogs, for example, N^{α}-alkylated amino acids such as MeGly (N^{α}-methylglycine), EtGly (N^{α}-ethylglycine) and EtAsn (N^{α}-ethylasparagine); and amino acids in which the α-carbon bears two side-chain substituents. As with the natural amino acids, the residues of the unnatural amino acids are what are left behind when the unnatural amino acid becomes part of a peptide sequence as described herein.

Amino acid residues are amino acid structures as described above that lack a hydrogen atom of the amino group or the hydroxyl moiety of the carboxyl group or both resulting in the units of a peptide chain,being amino-acid residues.

### Tethers (T)

T of Formula I is a lipohilic tether moiety which imparts lipophilicity to the CXCR5 receptor compounds of the invention. The lipophilicity which T imparts, can promote penetration of the CXCR5 receptor compounds into the cell membrane and tethering of the CXCR5 receptor compounds to the cell membrane. As such, the lipophilicity imparted by T can facilitate interaction between the CXCR5 receptor compounds of the invention and the cognate receptor.

The relative lipophilicity of compounds suitable for use as the lipophilic tether moiety of Formula I can be quantified by measuring the amount of the compound that partitions into an organic solvent layer (membrane-like) vs. an aqueous solvent layer (analogous to the extracellular or cytoplasmic environment). The partition coefficient in a mixed solvent composition, such as octanol/water or octanol/PBS, is the ratio of compound found at equilibrium in the octanol vs. the aqueous solvent (Partition coeff P=[compound]_{octnol}/[compound]_{aqueous}). Frequently, the partition coefficient is expressed in logarithmic form, as the log P. Compounds with greater lipophilicity have a more positive log P than more hydrophilic compounds and tend to interact more strongly with membrane bilayers.

Computational programs are also available for calculating the partition coefficient for compounds suitable for use as the lipophilic tether moiety (T). In situations where the chemical structure is being varied in a systematic manner, for example by adding additional methylene units (—CH₂—) onto to an existing alkyl group, the trend in log P can be calculateding, for example, ChemDraw (CambridgeSoft, Inc).

In one embodiment, T is an optionally substituted (C₆-C₃₀)alkyl, (C₆-C₃₀)alkenyl, (C₆-C₃₀)alkynyl wherein 0-3 carbon atoms are replaced with oxygen, sulfur, nitrogen or a combination thereof.

In a specific embodiment, the (C₆-C₃₀)alkyl, (C₆-C₃₀)alkenyl, (C₆-C₃₀)alkynyl are substituted at one or more substitutable carbon atoms with halogen, —CN, —OH, —NH₂, NO₂, —NH(C₁-C₆)alkyl, —N((C,-C₆)alkyl)₂, (C₁-C₆)alkyl, (C₁-C₆)haloalkyl, (C₁-C₆)alkoxy, (C₁-C₆)haloalkoxy, aryloxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)N((C₁-C₆)alkyl)₂, —N(C₁-C₆)alkylC(S)N((C₁-C₆)alkyl)₂, —CONH(C₁-C₆)alkyl, —OCONH(C₁-C₆)alkyl —CON((C₁-C₆)alkyl)₂, —C(S)(C₁-C₆)alkyl, —S(O)ₚ(C₁-C₆)alkyl, —S(O)ₚNH₂—S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H; and p is 1 or 2.

In a specific embodiment, T is selected from the group consisting of: CH₃(CH₂)₉OPh-, CH₃(CH₂)₆C═C(CH₂)₆, CH₃(CH₂)₁₁O(CH₂)₃, CH₃(CH₂)₉O(CH₂)₂ and CH₃(CH₂)₁₃.

In a specific embodiment, T is selected from the group consisting of: CH₃(CH₂)₁₆, CH₃(CH₂)₁₅, CH₃(CH₂)₁₄, CH₃(CH₂)₁₃ , CH₃(CH₂)₁₂, CH₃(CH₂)₁₁, CH₃(CH₂)₁₀, CH₃(CH₂)₉, CH₃(CH₂)₈, CH₃(CH₂)₉OPh-, CH₃(CH₂)₆C═C(CH₂)₆, CH₃(CH₂)₁₁O(CH₂)₃, and CH₃(CH₂)₉O(CH₂)₂ and CH₃(CH₂)₁₃.

It is understood that the lipophilic moiety (T) of Formula I can be derived from precursor liphophilic compounds (e.g., fatty acids and bile acids). As used herein, “derived from” with regard to T, means that T is derived from a precursor lipophilic compound and that reaction of the precursor lipophilic compound in preparing the CRF1 receptor compounds of Formula I, results in a lipophilic tether moiety represented by T in Formula I that is structurally modified in comparison to the precursor lipophilic compound.

For example, the lipophilic tether moiety, T of Formula I, can be derived from a fatty acid or a bile acid. It is understood that in accordance with Formula I, when T is derived from a fatty acid (i.e., a fatty acid derivative) it is attached to L-P at the carbon atom alpha to the carbonyl carbon of the acid functional group in the fatty acid from which it is derived. For example, when T is derived from palmitic acid,

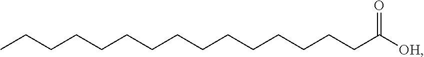

T of Formula I has the following structure:

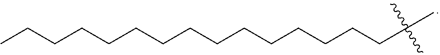

Similarly, when T is derived from stearic acid,

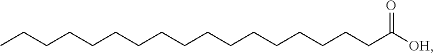

T of Formula I has the following structure:

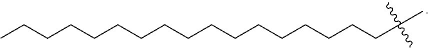

Similarly, when T is derived from 3-(dodecyloxy)propanoic acid,

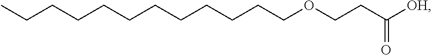

T of Formula I has the following structure:

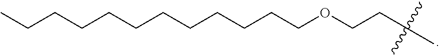

Similarly, when T is derived from 4-(undecyloxy)butanoic acid,

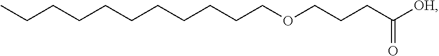

T of Formula I has the following structure:

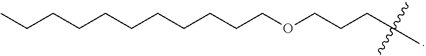

Similarly, when T is derived from elaidic acid,

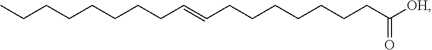

T of Formula I has the following structure:

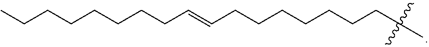

Similarly, when T is derived from oleic acid,

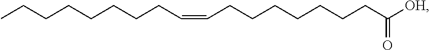

T of Formula I has the following structure:

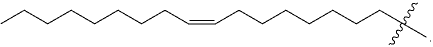

Similarly, when T is derived from 16-hydroxypalmitic acid,

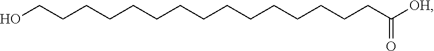

T of Formula I has the following structure:

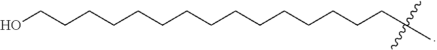

Similarly, when T is derived from 2-aminooctadecanoic acid

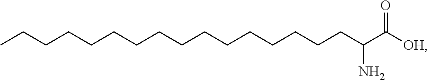

T of Formula I has the following structure:

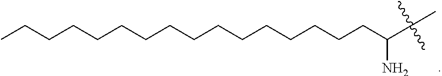

Similarly, when T is derived from 2-amino-4-(dodecyloxy)butanoic acid

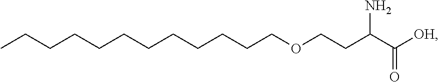

T of Formula I has the following structure:

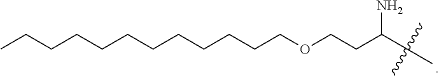

In a further embodiment, T is derived from a fatty acid. In a specific embodiment, T is derived from a fatty acid selected from the group consisting of: butyric acid, caproic acid, caprylic acid, capric acid, lauric acid, myristic acid, palmitic acid, stearic acid, arachidic acid, behenic acid, and lignoceric acid.

In another specific embodiment, T is derived from a fatty acid selected from the group consisting of: myristoleic acid, palmitoleic acid, oleic acid, linoleic acid, α-linolenic acid, arachidonic acid, eicosapentaenoic acid, erucic acid, docosahexaenoic acid.

In another embodiment, T of Formula I can be derived from a bile acid. Similar to the embodiment where T is a fatty acid derivative, it is understood that in accordance with Formula I, when T is derived from a bile acid (i.e., a bile acid derivative) it is attached to L-P at the carbon atom alpha to the carbonyl carbon of the acid functional group in the bile acid from which it is derived. For example, when T is derived from lithocholic acid,

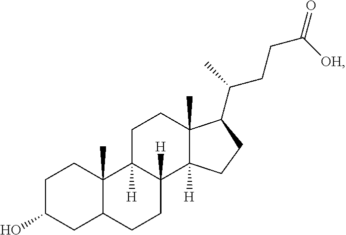

T of Formula I has the following structure:

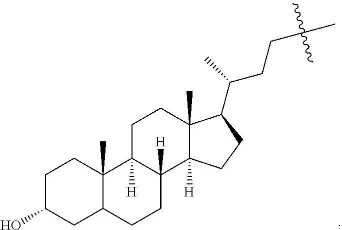

In a further embodiment, T is derived from a bile acid. In a specific embodiment, T is derived from a bile acid selected from the group consisting of: lithocholic acid, chenodeoxycholic acid, deoxycholic acid, cholanic acid, cholic acid, ursocholic acid, ursodeoxycholic acid, isoursodeoxycholic acid, lagodeoxycholic acid, dehydrocholic acid, hyocholic acid, hyodeoxycholic acid and the like.

For example, T is selected from:

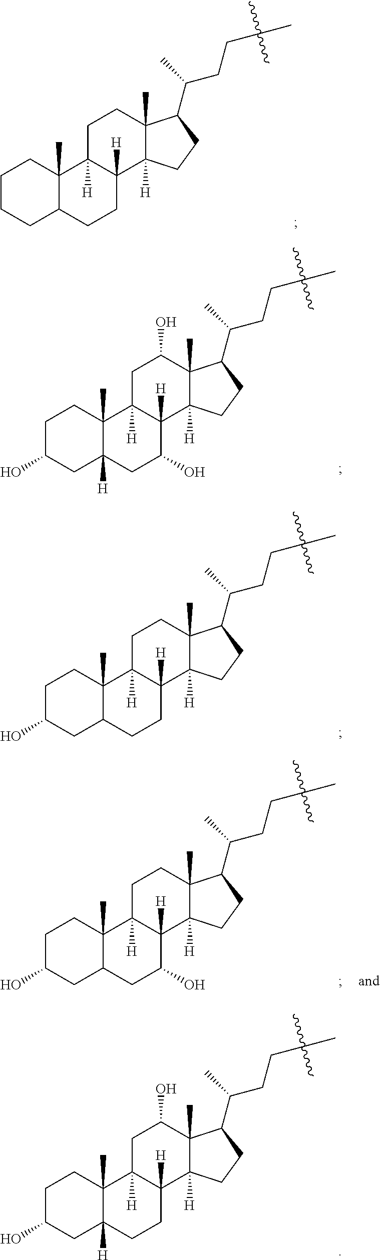

In another further embodiment, T is derived from a bile acid described above that has been modified at other than the acid functional group. For example, T can be derived from any of the bile acids described above, where the hydroxy position has been modified to form an ester or a halo ester. For example, T can be:

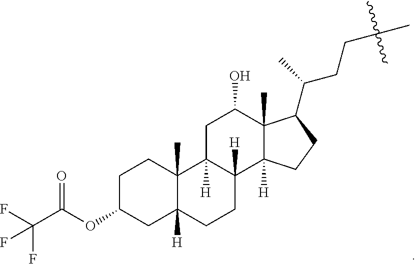

Other lipophilic moieties suitable for use as the lipophilic membrane tether, T, of Formula I, include but are not limited to steroids. Suitable steroids include, but are not limited to, sterols; progestagens; glucocorticoids; mineralcorticoids; androgens; and estrogens. Generally any steroid capable of attachment or which can be modified for incorporation into Formula I can be used. It is understood that the lipophilic membrane tether, T, may be slightly modified from the precursor lipophilic compound as a result of incorporation into Formula I.

Suitable sterols for use in the invention at T, include but are not limited to: cholestanol, coprostanol, cholesterol, epicholesterol, ergosterol, ergocalciferol, and the like. Preferred sterols are those that provide a balance of lipophilicity with water solubility.

Suitable progestagens include, but are not limited to progesterone. Suitable glucocorticoids include, but are not limited to cortisol. Suitable mineralcorticoids include, but are not limited to aldosterone. Suitable androgens include, but are not limited to testosterone and androstenedione. Suitable estrogens include, but are not limited to estrone and estradiol.

In another specific embodiment, T can be derived from 2-tetradecanamideooctadecanoid acid. Similar to the embodiment where T is a fatty acid derivative, it is understood that in accordance with Formula I, when T is derived from 2-tetradecanamideooctadecanoid acid it is attached to L-P at the carbon atom alpha to the carbonyl carbon of the acid functional group in the bile acid from which it is derived. For example, when T is derived from 2-tetradecanamideooctadecanoid acid, the tether is:

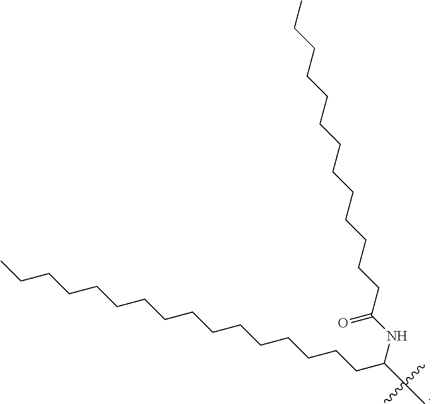

In another embodiment, T of Formula I can be derived from 2-(5-((3aS,4S,6aR)-2-oxohexahydro-1H-thieno[3,4-d]imidazol-4-yl)pentanamido)octadecanoic acid. For example, when T is derived from 2-(5-((3aS,4S,6aR)-2-oxohexahydro-1H-thieno[3,4-d]imidazol-4-yl)pentanamido)octadecanoic acid, the tether is:

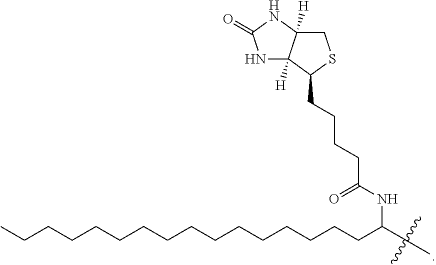

In yet another embodiment, T of Formula I can be:

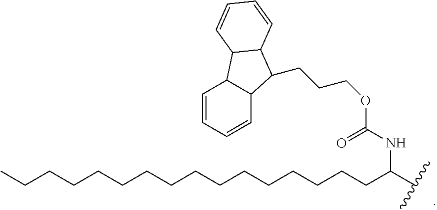

It is understood, that the compounds can contain one of more tether moieties.

In certain aspects, the tether moieties are the same. In other embodiments, the tether moieties are different.

### Compounds (T-L-P)

In a first aspect, the GPCR Compound of the invention is represented by Formula I:

T-L-P,
or pharmaceutically acceptable salts thereof, wherein:
   P is a peptide comprising at least three contiguous amino-acid residues
of an intracellular i1, i2, i3 loop or an intracellular i4 domain of the CXCR5 receptor;
   L is a linking moiety represented by C(O) and bonded to P at an N terminal nitrogen of an N-terminal amino-acid residue;
   and T is a lipophilic tether moiety bonded to L.

In a second aspect, P comprises at least six contiguous amino acid residues.

In a third aspect, P comprises at least 3 contiguous amino acids of the i1 loop.

In a specific embodiment of the third aspect, the i1 loop of the CXCR5 receptor from which P is derived has the following sequence:

| | |
|---|---|
| LVILERHRQTRSSTETFLFH. | (SEQ ID NO: 94) |

In another embodiment of the third aspect, P is a sequence selected from:

TABLE 6

| CXCR5 | | |
|---|---|---|
| i-Loop | Sequence | SEQ ID: |
| i1 | LVILERHRQTRSS | 1 |
| i1 | LERHRQTRSSTE | 2 |
| i1 | RHRQTRSSTETFLFH | 3 |
| i1 | RHRQTRSSTET | 4 |
| i1 | RQTRSSTETFL | 5 |
| i1 | RQTRSSTETF | 6 |
| i1 | ERHRQTRSSTE | 7 |
| i1 | RHRQTRSSTE | 8 |
| i1 | RHRQTRSSTET | 9 |
| i1 | LERHRQTRSSTETFL | 10 |
| i1 | RQTRSSTE | 11 |
| i1 | RQTRSSTET | 12 |
| i1 | HRQTRSSTE | 13 |

It is understood that for the embodiments presented herein, that when the amino acid residues of P are represented by X, W, Y or Z that the C-terminal amino acid residue does not include the —OH of the amino acid and that the end group R₁ that is bonded to the C-terminal residue includes —OH as well as other moieties defined herein.

In one embodiment, the CXCR5 compounds of the invention are represented by Formula A:

T-L-X1-X2-X3-X4-X5-X6-X7-X8-X9-X10-X11-X12-X13-
X14-X15-X16-X₁₇-X₁₈-X₁₉-X₂₀-R_{1;}

or a pharmaceutically acceptable salts thereof, wherein: L is a linking moiety represented by C(O) and bonded to the N terminal nitrogen of X₁ or the next present amino acid residue if X₁ is not present; T is a lipophilic tether moiety bonded to L; and R₁ is OR₂ or N(R₂)₂, wherein each R₂ is independently H or alkyl,
wherein at least three contiguous X₁-X₂₄ amino acid residues are present, and wherein:
   X₁ is a leucine residue or absent,
   X₂ is a valine residue or absent,
   X₃ is isoleucine or absent,
   X₄ is a leucine residue or absent,
   X₅ is a glutamic acid residue or absent,
   X₆ is a arginine residue or absent,
   X₇ is a histidine residue or absent,
   X₈ is a arginine residue,
   X₉ is a glutamine residue,
   X₁₀ is a threonine residue,
   X₁₁ is a arginine residue,
   X₁₂ is a serine residue,
   X₁₃ is a serine residue,
   X₁₄ is a threonine residue or absent,
   X₁₅ is a glutamic acid residue or absent,
   X₁₆ is a threonine residue or absent,
   X₁₇ is a phenylalanine residue or absent,
   X₁₈ is a leucine residue or absent,
   X₁₉ is a phenylalanine residue or absent,
   X₂₀ is a histidine residue or absent.

In a more specific embodiment, X₁₆-X₂₀ are absent and wherein X₁₄ is a threonine residue, and X₁₅ is a glutamic acid residue. In yet another embodiment, X₁-X₆, X₁₉ and X₂₀ are absent or X₁-X₃ are absent.

In another embodiment, the CXCR5 compounds of the invention comprise wherein:
X₁ is a leucine residue,
X₂ is a valine residue,
X₃ is isoleucine residue,
X₄ is a leucine residue,
X₅ is a glutamic acid residue,
X₆ is a arginine residue,
X₇ is a histidine residue,
X₁₄ is a threonine residue or absent,
X₁₅ is a glutamic acid residue or absent,
X₁₆ is a threonine residue or absent,
X₁₇ is a phenylalanine residue or absent,
X₁₈ is a leucine residue or absent,
X₁₉ is a phenylalanine residue or absent, and
X₂₀ is a histidine residue or absent and optionally further comprising when X₁₄-X₂₀ is absent.

In a more specific embodiment, the CXCR5 compounds of Formula A are selected from:

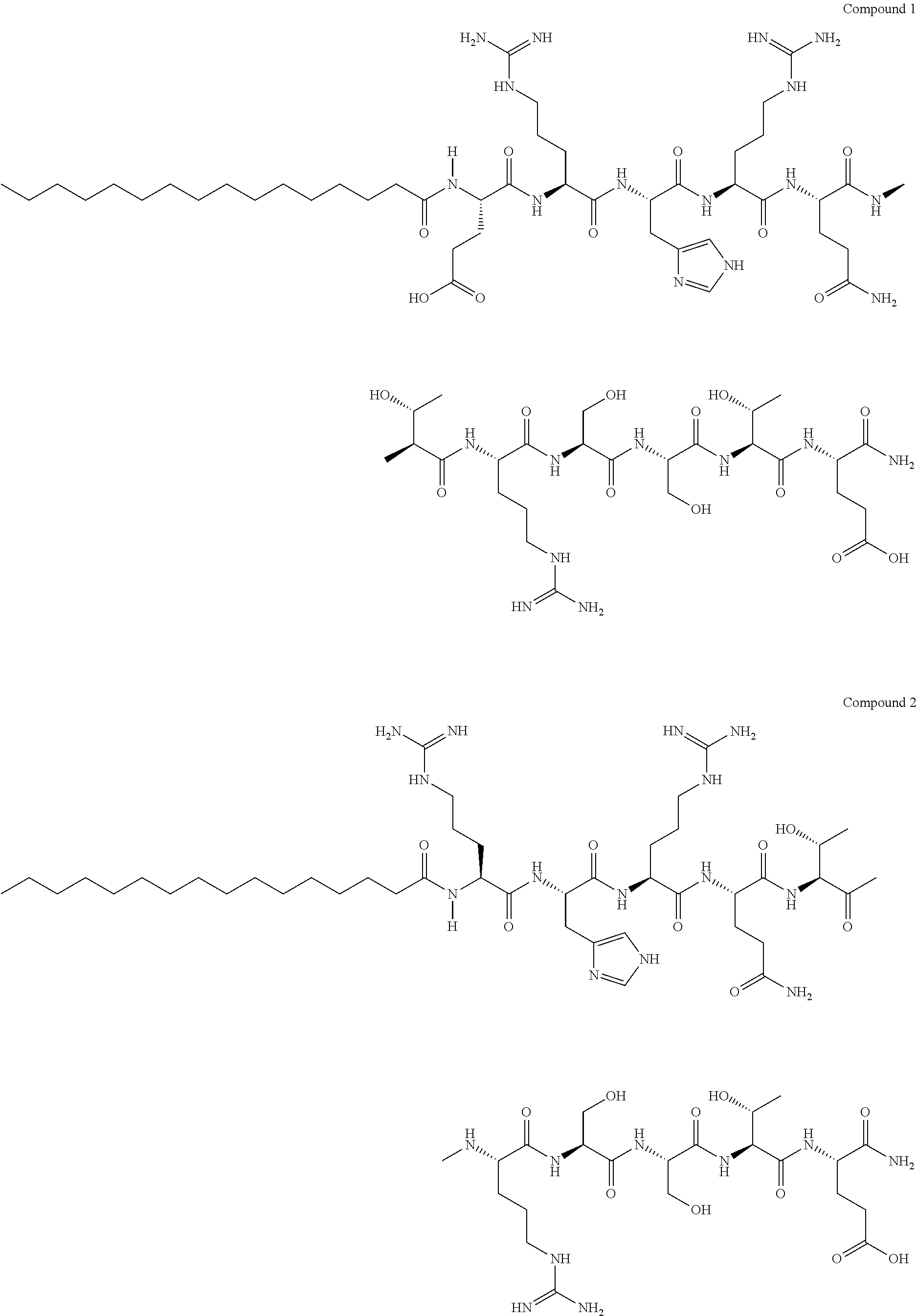

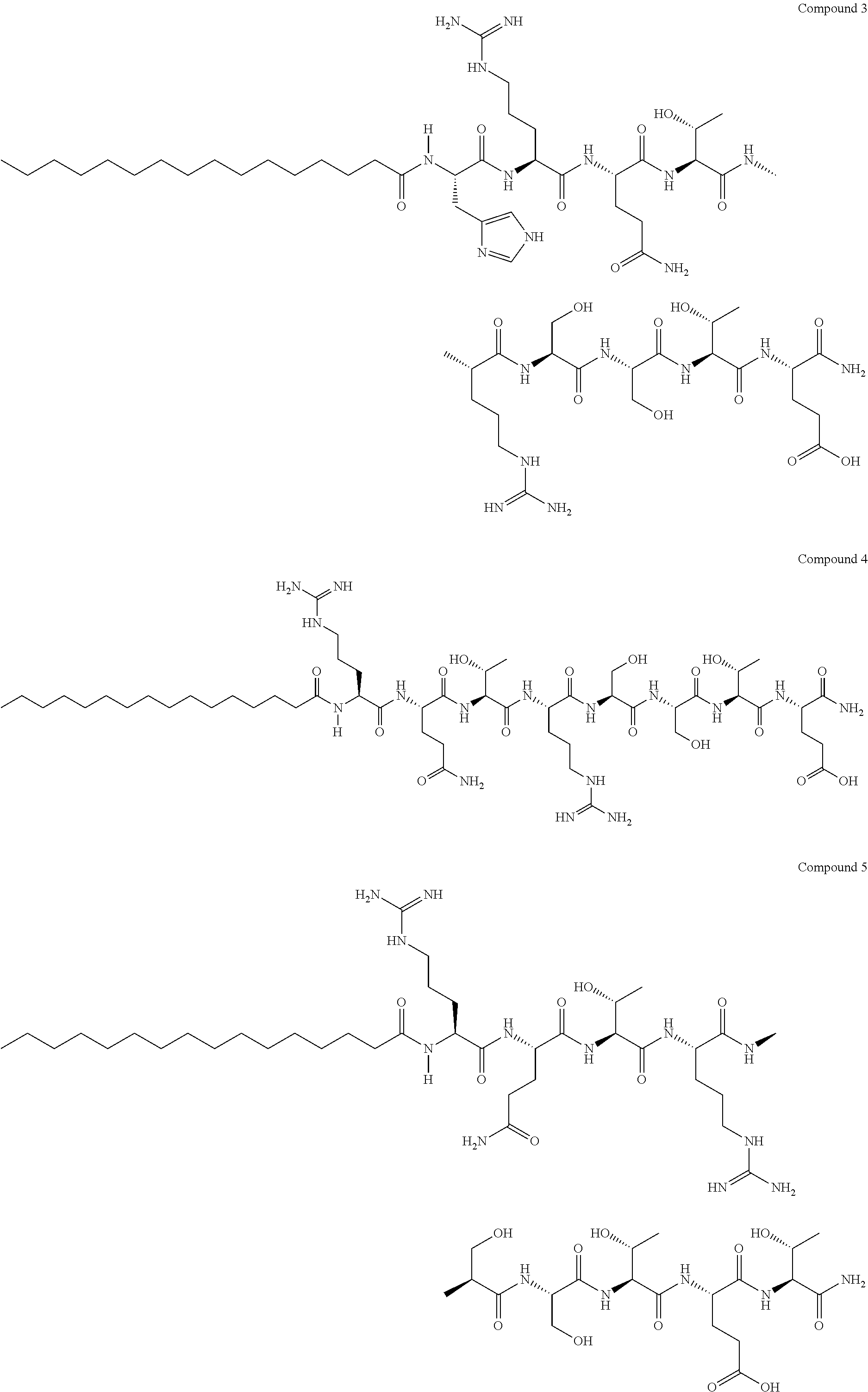

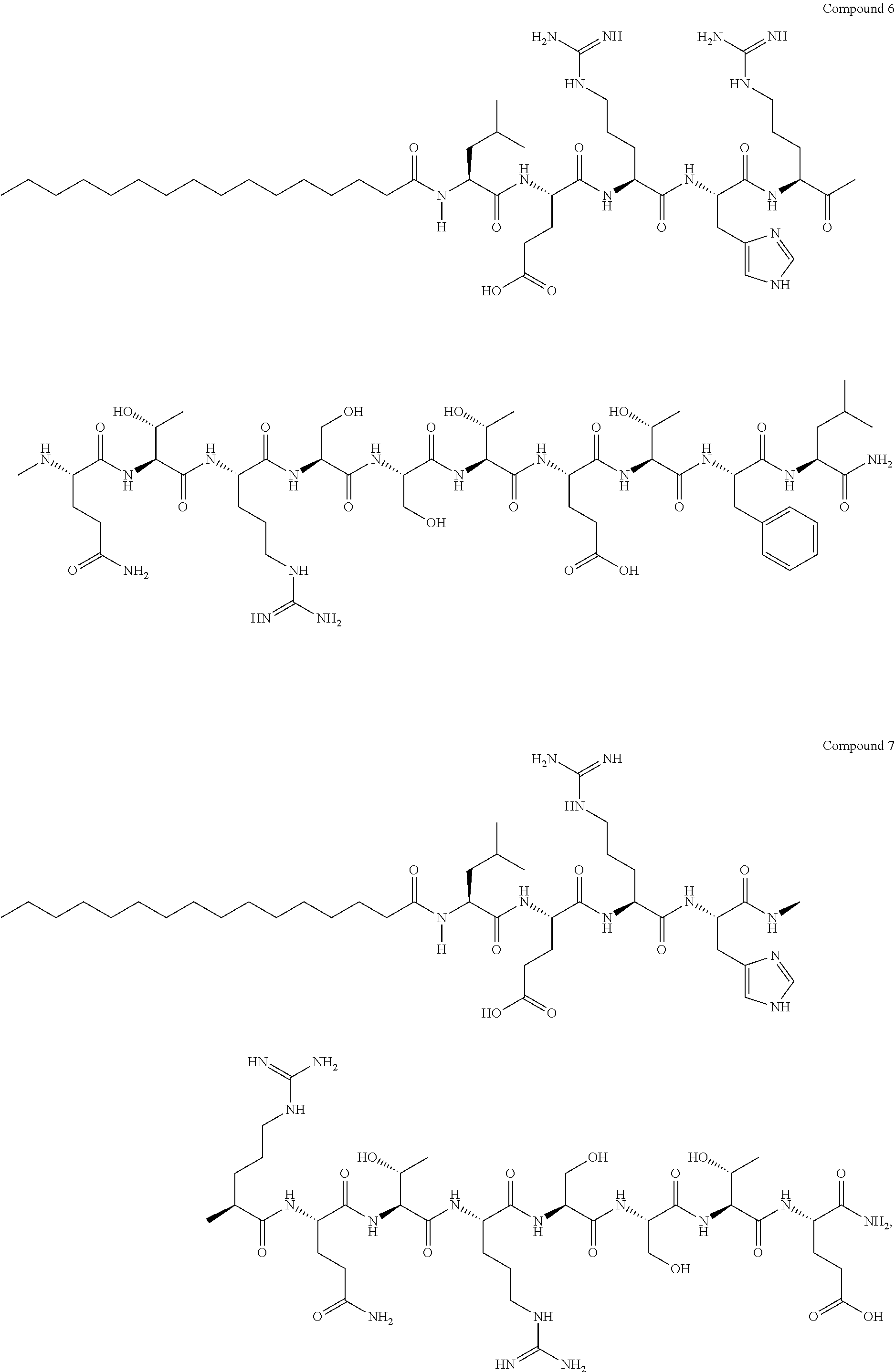

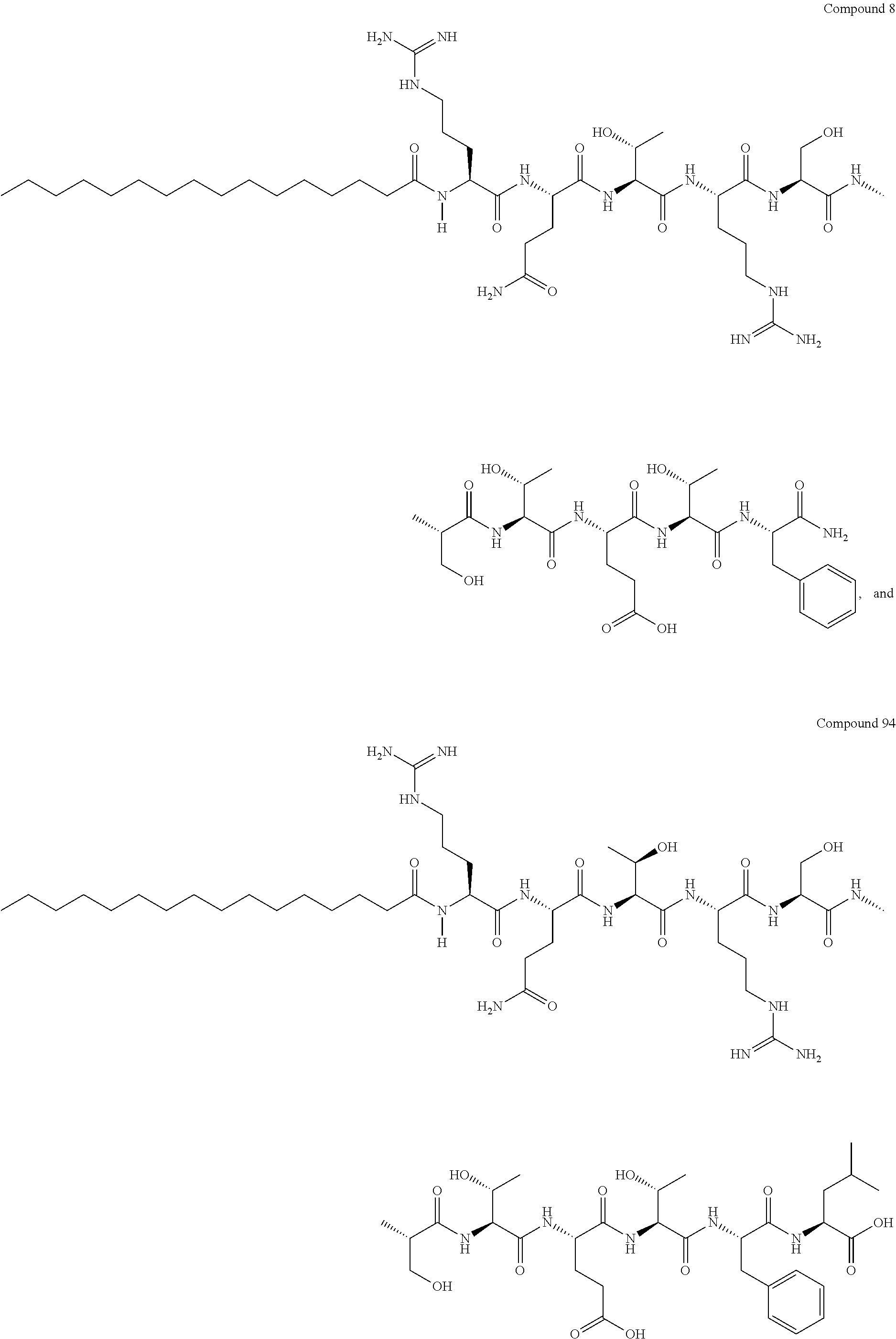

or a pharmaceutically acceptable salt of any of the foregoing.

In a fourth aspect, P comprises at least 3 contiguous amino acids of the i2 loop.

In a specific embodiment of the fourth aspect, the i2 loop of the CXCR5 receptor from which P is derived has the following sequence:

| | |
|---|---|
| LAIVHAVHAYRHRRLLSIHIT. | (SEQ ID NO: 95) |

In another embodiment of the fourth aspect, P is a sequence selected from:

TABLE 7

| CXCR5 | | |
|---|---|---|
| i-Loop | Sequence | SEQ ID: |
| i2 | LAIVHAVHAYRHRRLLSIHIT | 14 |
| i2 | AIVHAVHAYRHRRLLSI | 15 |
| i2 | IVHAVHAYRHRR | 16 |
| i2 | VHAVHAYRHRR | 17 |
| i2 | HAVHAYRHRR | 18 |
| i2 | HAVHAYRHRRLLSI | 19 |
| i2 | HAVHAYRHRRLLS | 20 |
| i2 | HAVHAYRHRRLL | 21 |
| i2 | HAVHAYRHRRL | 22 |
| i2 | YRHRRLLSIH | 23 |
| i2 | RHRRLLSIHIT | 24 |
| i2 | RHRRLLSIH | 25 |
| i2 | HAYRHRRLLSI | 26 |
| i2 | VHAYRHRRLLSI | 27 |
| i2 | AVHAYRHRRLLSI | 28 |
| i2 | AAVHAYRHRRLLSI | 29 |
| i2 | HAAVHAYRHRRLLSI | 30 |
| i2 | IVHAVHAYRHRRLLSI | 31 |
| i2 | VHAVHAYRHRRLLSI | 32 |
| i2 | hAVHAYRHRRLLSI | 33 |
| i2 | HaVHAYRHRRLLSI | 34 |
| i2 | HAhAYRHRRLLSI | 35 |
| i2 | HAvHAYRHRRLLSI | 36 |
| i2 | HAVAAYRHRRLLSI | 37 |
| i2 | HAVHAARHRRLLSI | 38 |
| i2 | HAVHAYAHRRLLSI | 39 |
| i2 | HAVHAYRARRLLSI | 40 |
| i2 | HAVHAYRHARLLSI | 41 |
| i2 | HAVHAYRHRALLSI | 42 |
| i2 | HAVHAYRHRRALSI | 43 |
| i2 | HAVHAYRHRRLASI | 44 |
| i2 | HAVHAYRHRRLLAI | 45 |
| i2 | HAVHAYRHRRLLSA | 46 |
| i2 | HAVhAYRHRRLLSI | 47 |
| i2 | HAVHaYRHRRLLSI | 48 |
| i2 | HAVHAyRHRRLLSI | 49 |
| i2 | HAVHAYrHRRLLSI | 50 |
| i2 | HAVHAYRhRRLLSI | 51 |
| i2 | HAVHAYRHrRLLSI | 52 |
| i2 | HAVHAYRHRrLLSI | 53 |
| i2 | HAVHAYRHRRILSI | 54 |
| i2 | HAVHAYRHRRLISI | 55 |
| i2 | HAVHAYRHRRLLsI | 56 |
| i2 | HAVHAYRHRRLLFI | 57 |
| i2 | HAVHAYRHRRLLVI | 58 |
| i2 | HAVHAYRRRRLLS | 59 |
| i2 | HAVHAYRRRRLLSI | 60 |

In another embodiment, the CXCR5 compounds represented by Formula B or a pharmaceutically acceptable salts thereof comprise, wherein:

T-L-Y₁-Y₂-Y₃-Y₄-Y₅-Y₆-Y₇-Y₈-Y₉-Y₁₀-Y₁₁-Y₁₂-Y₁₃-
Y₁₄-Y₁₅-Y₁₆-Y₁₇-Y₁₈-Y₁₉-Y₂₀-Y₂₁-Y₂₂-Y₂₃-Y₂₄-Y₂₅-R_{1;}

wherein L is a linking moiety represented by C(O) and bonded the N terminal nitrogen of Y₁ or the next present amino acid residue if Y₁ is absent, T is a lipophilic tether moiety bonded to L; and R₁ is OR₂ or N(R₂)₂, wherein each R₂ is independently H or alkyl,
wherein at least three contiguous Y₁-Y₂₅ amino acid residues are present, and wherein:
   Y₁ is a leucine residue or absent,
   Y₂ is a alanine residue or absent,
   Y₃ is a isoleucine residue or absent,
   Y₄ is a valine residue or absent,
   Y₅ is a histidine residue, alanine residue or absent,
   Y₆ is a alanine residue or absent,
   Y₇ is a valine residue or absent,
   Y₈ is a histidine residue,
   Y₉ is a alanine residue,
   Y₁₀ is a tyrosine residue,
   Y₁₁ is a arginine residue,
   Y₁₂ is a histidine residue,
   Y₁₃ is a arginine residue,
   Y₁₄ is a arginine residue,
   Y₁₅ is a leucine residue or absent,
   Y₁₆ is a leucine residue or absent,
   Y₁₇ is a serine residue or absent,
   Y₁₈ is a isoleucine residue or absent,
   Y₁₉ is a histidine residue or absent,
   Y₂₀ is a isoleucine residue or'absent, and
   Y₂₁ is a threonine or residue absent.

In a more specific embodiment, Y₁, Y₂, and Y₁₅-Y₂₁ are absent and Y₅ is histadine or Y₁-Y₄ and Y_{19-Y}₂₁ are absent and/or Y₅ is a histadine residue. In another embodiment, Y₅ is absent or an alanine residue.

In a more specific embodiment, the amino acid residues Y₁-Y₂₁ are present.

In another more specific embodiment, the CXCR5 compounds of Formula B are selected from: Compounds 9-56 or a pharmaceutically salt thereof of any of the forgoing compounds.

In a fifth aspect, P comprises at least 3 contiguous amino acids of the i3 loop.

In a specific embodiment of the fifth aspect, the i3 loop of the CXCR5 receptor from which P is derived has the following sequence:

| | |
|---|---|
| VVHRLRQAQRRPQRQKAVRVA. | (SEQ ID NO: 96) |

In another embodiment of the fifth aspect, P is a sequence selected from:

TABLE 8

| CXCR5 | | |
|---|---|---|
| i-Loop | Sequence | SEQ ID: |
| i3 | VVHRLRQAQRRPQRQKAVRVA | 61 |
| i3 | RQAQRRPQRQK | 62 |
| i3 | RQAQRRPQRQKAVRVAI | 63 |
| i3 | RLRQAQRRPQRQK | 64 |
| i3 | RQAQRRPQRQKAV | 65 |
| i3 | GVVHRLRQAQRRPQRQK | 66 |
| i3 | GVVHRLRQAQRRPQRQKAVRVAI | 67 |
| i3 | VHRLRQAQRRPQRQK | 68 |
| i3 | RLRQAQRRPQRQKAVR | 69 |
| i3 | RLRQAQRRPQR | 70 |
| i3 | RLRQAQRRPQRQKAV | 71 |
| i3 | RQAQRRPQRQKAVRV | 72 |
| i3 | RRPQRQKAVR | 73 |
| i3 | HFRKERIEGLRKRRRL | 74 |
| i3 | RPQRQKAVRVAI | 75 |
| i3 | RQAQRRPQRQKAVR | 76 |
| i3 | RQAQRRPQRQKAVRA | 77 |
| i3 | RLRNANRRPNRNKAVRV | 78 |
| i3 | RLRQAQRRPQR | 79 |
| i3 | RLRQAQRRPQRQ | 80 |
| i3 | RLRQAQRRPQRQKA | 81 |
| i3 | RLRQAQRRPQRQKAVRV | 82 |
| i3 | RLRTATRRPTRTKAVRV | 83 |
| i3 | GRLRQAQRRPQRQK | 84 |
| i3 | HRLRQAQRRPQR | 85 |
| i3 | HRLRQAQRRPQRQK | 86 |
| i3 | GSGRLRQAQRRPQRQKAVRV | 87 |

In another embodiment, the CXCR5 compounds represented by Formula C or a pharmaceutically acceptable salts thereof, comprise:

T-L-W₁-W₂-W₃-W₄-W₅-W₆-W₇-W₈-W₉-W₁₀-W₁₁-W₁₂-W₁₃-W₁₄-
W₁₅-W₁₆-W₁₇-W₁₈-W₁₉-W₂₀-W₂₁-W₂₂-W₂₃-R_{1;}

wherein L is a linking moiety represented by C(O) and bonded to the N terminal nitrogen of W₁ or the next present amino acid residue if W₁ is absent; T is a lipophilic tether moiety bonded to L; and R₁ is OR₂ or N(R₂)₂, wherein each R₂ is independently H or alkyl, wherein at least three contiguous W₁-W₂₃ amino acid residues are present and wherein:
W₁ is a glycine residue, a histidine residue or absent,
W₂ is a valine, phenylalanine residue, glycine residue or absent,
W₃ is a valine residue, arginine residue, serine residue or absent,
W₄ is a histidine residue, lysine residue, glycine residue or absent,
W₅ is a arginine residue, glutamic residue acid or absent,
W₆ is a leucine residue, arginine residue or absent,
W₇ is a arginine residue, isoleucine residue or absent,
W₈ is a glutamine residue, glutamic acid residue, asparagine residue, threonine residue or absent,
W₉ is a alanine residue, glycine residue or absent,
W₁₀ is a glutamine residue, leucine residue, asparagine residue, or a threonine residue or absent,
W₁₁ is a arginine residue or absent,
W₁₂ is a arginine residue or lysine,
W₁₃ is a proline residue or arginine,
W₁₄ is a glutamine residue, a arginine residue, a asparagine residue or a threonine residue,
W₁₅ is a arginine residue,
W₁₆ is a glutamine residue, a leucine residue, an asparagine residue, a threonine residue or absent,
W₁₇ is a lysine residue or absent,
W₁₈ is an alanine residue or absent,
W₁₉ is a valine residue or absent,
W₂₀ is a arginine residue or absent,
W₂₁ is a valine residue or absent,
W₂₂ is a alanine residue or absent, and
W₂₃ is a isoleucine residue or absent.

In another embodiment of a CXCR5 compound of Formula C,
W₁ is a glycine residue or absent,
W₂ is a valine residue or absent,
W₃ is a valine residue or absent,
W₄ is a histidine residue or absent,
W₅ is a arginine residue or absent,
W₆ is a leucine residue or absent,
W₇ is a arginine residue, or absent,
W₈ is a glutamine residue, or absent,
W₉ is a alanine residue or absent,
W₁₀ is a glutamine residue or absent,
W₁₁ is a arginine residue or absent,
W₁₂ is a arginine residue,
W₁₃ is a proline residue,
W₁₄ is a glutamine residue,
W₁₅ is a arginine residue,
W₁₆ is a glutamine residue, or absent,
W₁₇ is a lysine residue or absent,
W₁₈ is an alanine residue or absent,
W₁₉ is a valine residue or absent,
W₂₀ is a arginine residue or absent,
W₂₁ is a valine residue or absent,
W₂₂ is a alanine residue or absent, and
W₂₃ is a isoleucine residue or absent.

In another embodiment of Formula C, W₁₈-W₂₃ are absentor W₁-W₄ are absent and W₅ is an arginine residue.

In a more specific embodiment of Formula C, the CXCR5 compound is selected from compounds 57-79 or a pharmaceutically acceptable salt thereof.

In a sixth aspect, P comprises at least 3 contiguous amino acids of the i4 domain.

In a specific embodiment of the sixth aspect, the i4 domain of the CXCR5 receptor from which P is derived has the following sequence:

(SEQ ID NO: 97)
AGVKFRSDLSRLLTKLGCTGPASLCQLFPSWRRSSLSESENATSLTTF.

In another embodiment of the sixth aspect, P is a sequence selected from:

TABLE 9

| CXCR5 | | |
|---|---|---|
| i-Loop | Sequence | SEQ ID: |
| i4 | AGVKFRSDLSRLLTKLG | 88 |
| i4 | RSDLSRLLTKLGS | 89 |
| i4 | RSDLSRLLTK | 90 |
| i4 | LLTKLGSTGPASL | 91 |
| i4 | LSRLLTKLGSTGP | 92 |
| i4 | VKFRSDLSRLLTKLGSTGPASLS | 93 |

In another embodiment a CXCR5 compound represented is by Formula D or a pharmaceutically acceptable salts thereof:

T-L-Z₁-Z₂-Z₃-Z₄-Z₅-Z₆-Z₇-Z₈-Z₉-Z₁₀-Z₁₁-Z₁₂-Z₁₃-Z₁₄-Z₁₅-
Z₁₆-Z₁₇-Z₁₈-Z₁₉-Z₂₀-Z₂₁-Z₂₂-Z₂₃-Z₂₃-Z₂₄-Z₂₅-Z₂₆-Z₂₇-Z₂₈-
Z₂₉-Z₃₀-Z₃₁Z₃₂-Z₃₃-Z₃₄-Z₃₅-Z₃₆-Z₃₇-Z₃₈-Z₃₉-Z₄₀-Z₄₁-Z₄₂-Z₄₃-
Z₄₄-Z₄₅-Z₄₆-Z₄₇-Z₄₈-R₁;

wherein L is a linking moiety represented by C(O) and bonded to the N terminal nitrogen of Z₁ or the next present amino acid if Z₁ is absent; T is a lipophilic tether moiety bonded to L; and R₁ is OR₂ or N(R₂)₂, wherein each R₂ is independently H or alkyl, wherein at least three contiguous Z₁-Z₂₃ amino acid residues are present and wherein:
   Z₁ is a alanine residue or absent,
   Z₂ is a glycine residue or absent,
   Z₃ is a valine residue, or absent,
   Z₄ is a lysine residue or absent,
   Z₅ is a phenylalanine residue or absent,
   Z₆ is an arginine residue or absent,
   Z₇ is a serine residue or absent,
   Z₈ is a aspartic acid residue or absent,
   Z₉ is a leucine residue or absent,
   Z₁₀ is a serine residue or absent,
   Z₁₁ is a arginine residue or absent,
   Z₁₂ is a leucine residue,
   Z₁₃ is a leucine residue or arginine,
   Z₁₄ is a threonine residue,
   Z₁₅ is a lysine residue,
   Z₁₆ is a leucine residue or absent,
   Z₁₇ is a glycine residue or absent,
   Z₁₈ is a cysteine residue, a serine residue or absent,
   Z₁₉ is a threonine residue or absent,
   Z₂₀ is a glycine or absent residue or absent,
   Z₂₁ is a proline residue or absent,
   Z₂₂ is a alanine residue or absent,
   Z₂₃ is a serine residue or absent,
   Z₂₄ is a leucine residue or absent,
   Z₂₅ is a cysteine residue, a serine residue or absent,
   Z₂₆ is a glutamine residue or absent,
   Z₂₇ is a leucine residue or absent,
   Z₂₈ is a phenylalanine residue or absent,
   Z₂₉ is a proline residue or absent,
   Z₃₀ is a serine residue or absent,
   Z₃₁ is a tryptophan residue or absent,
   Z₃₂ is a arginine residue or absent,
   Z₃₃ is a arginine residue or absent,
   Z₃₄ is a serine residue or absent,
   Z₃₅ is a serine residue or absent,
   Z₃₆ is a leucine residue or absent,
   Z₃₇ is a serine residue or absent,
   Z₃₈ is a glutamic residue acid or absent,
   Z₃₉ is a serine residue or absent,
   Z₄₀ is a glutamic residue acid or absent,
   Z₄₁ is a asparagine residue or absent,
   Z₄₂ is a alanine residue or absent,
   Z₄₃ is a threonine residue or absent,
   Z₄₄ is a serine residue or absent,
   Z₄₅ is a leucine residue or absent,
   Z₄₆ is a threonine residue or absent,
   Z₄₇ is a threonine residue or absent, and
   Z₄₈ is a phenylalanine residue or absent.

In other embodiments of Formula D, Z₂₆-Z₄₈ is absent or Z₁₈-Z₄₈ is absent or Z₁₆-Z₄₈ is absent or Z₁-Z₁₁ is absent or Z₁-Z₈ is absent or Z₁-Z₅ is absent.

In a more specific embodiment, a compound of Formula D is selected from compounds 80-83 or a pharmaceutically acceptable salt thereof.

In a seventh aspect, T is an optionally substituted (C₆-C₃₀)alkyl, (C₆-C₃₀)alkenyl, (C₆-C₃₀)alkynyl, wherein 0-3 carbon atoms are replaced with oxygen, sulfur, nitrogen or a combination thereof. This value of T is applicable to the first, second, third, fourth, fifth and sixth aspects and the specific (i.e., specific, more specific and most specific) embodiments of same.

In a specific embodiment of the seventh aspect, T is selected from: CH₃(CH₂)₁₆, CH₃(CH₂)_{15,} CH₃(CH₂)₁₄, CH₃(CH₂)₁₃ , CH₃(CH₂)₁₂, CH₃(CH₂)₁₁, CH₃(CH₂)₁₀, CH₃(CH₂)⁹, CH₃(CH₂)₈, CH₃(CH₂)₉OPh-, CH₃(CH₂)₆C═C(CH₂)₆. CH₃(CH₂)₁₁ O(CH₂)₃, and CH₃(CH₂)₉O(CH₂)₂.

In another specific embodiment of the seventh aspect, T is a fatty acid derivative.

In a more specific embodiment of the seventh aspect, the fatty acid is selected from the group consisting of: butyric acid, caproic acid, caprylic acid, capric acid, lauric acid, myristic acid, palmitic acid, stearic acid, arachidic acid, behenic acid, lignoceric acid, myristoleic acid, palmitoleic acid, oleic acid, linoleic acid, α-linolenic acid, arachidonic acid, eicosapentaenoic acid, erucic acid, docosahexaenoic acid.

In an eighth aspect, T is a bile acid derivative. This value of T is applicable to the first, second, third, fourth, fifth and sixth aspects and the specific (i.e., specific, more specific and most specific) embodiments of same.

In a specific embodiment of the eighth aspect, the bile acid is selected from the group consisting of: lithocholic acid, chenodeoxycholic acid, deoxycholic acid, cholanic acid, cholic acid, ursocholic acid, ursodeoxycholic acid, isoursodeoxycholic acid, lagodeoxycholic acid, dehydrocholic acid, hyocholic acid, and hyodeoxycholic acid.

In a ninth aspect, T is selected from sterols; progestagens; glucocorticoids; mineralcorticoids; androgens; and estrogens. This value of T is applicable to the first, second, third, fourth, fifth and sixth aspects and the specific (i.e., specific, more specific and most specific) embodiments of same.

In a tenth aspect, T-L of Formula I is represented by a moiety selected from the group consisting of:
CH₃(CH₂)₁₅—C(O);
CH₃(CH₂)₁₃—C(O);
CH₃(CH₂)₉O(CH₂)₂C(O);
CH₃(CH₂)₁₀O(CH₂)₂C(O);
CH₃(CH₂)₆C═C(CH₂)₆—C(O);
LCA-C(O); and
CH₃(CH₂)₉OPh-C(O) wherein

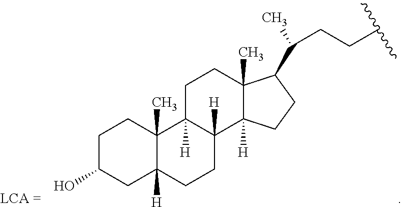

In an eleventh aspect, the CXCR5 receptor Compounds of Formula I, can also have a linker (L)-tether (T) moiety bonded to P at: a nitrogen-containing side chain of an amino acid residue of P; an oxygen-containing or sulfur-containing side chain of an amino acid residue of P; at the carbonyl carbon of the c-terminal amino acid residue; or a combination of any of the foregoing.

In a twelfth aspect, T of Formula I is,represented by a moiety selected from the group consisting of:

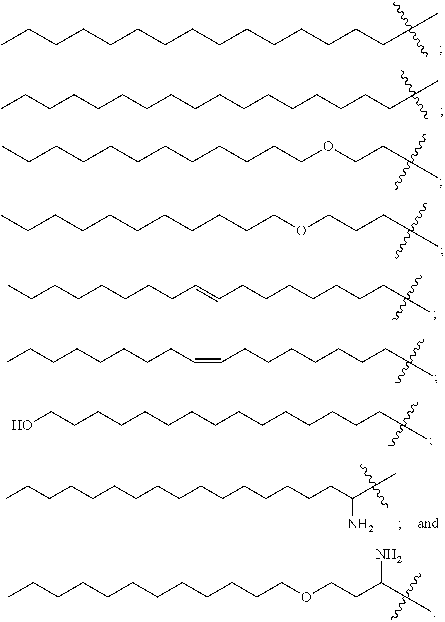

In a thirteenth aspect, the CXCR5 receptor compounds are selected from the compounds presented in Tables 10-17 or pharmaceutically acceptable salts thereof, excluding controls and those compounds not within the structure of Formula 1.

TABLE 10
CXCR5 i1 loop compounds (amide on C-terminus)

| | | | | | | Mol. |
|---|---|---|---|---|---|---|
| Comp. # | Loop | Sequence | N terminus | MS Theoretical | MS Observed | Weight |
| 1 | i1 | ERHRQTRSSTE | C₁₅H₃₁C(O)— | 542.285 | 542.2 | 1623.855 |
| | | (SEQ ID NO. 7) | | | | |
| 2 | i1 | RHRQTRSSTE | C₁₅H₃₁C(O)— | 499.247 | 499.1 | 1494.741 |
| | | (SEQ ID NO. 8) | | | | |
| 3 | i1 | HRQTRSSTE | C₁₅H₃₁C(O)— | 670.278 | 570.5 | 1338.556 |
| | | (SEQ ID NO. 13) | | | | |
| 4 | i1 | RQTRSSTE | C₁₅H₃₁C(O)— | 601.708 | 601.5 | 1201.416 |
| | | (SEQ ID NO. 11) | | | | |
| 5 | i1 | RQTRSSTET | C₁₅H₃₁C(O)— | 652.26 | 652 | 1302.52 |
| | | (SEQ ID NO. 12) | | | | |
| 6 | i1 | LERHRQTRSSTETFL | C₁₅H₃₁C(O)— | 700.48 | 700.1 | 2098.448 |
| | | (SEQ ID NO. 10) | | | | |
| 7 | i1 | LERHRQTRSSTE | C₁₅H₃₁C(O)— | 580.00 | 579.5 | 1737.013 |
| | | (SEQ ID NO 2) | | | | |
| 8 | i1 | RQTRSSTETF | C₁₅H₃₁C(O)— | 1449.84 | 1449.39 | 1449.694 |
| | | (SEQ ID NO. 6) | | | | |
| 94 | i1 | RQTRSSTETFL | C₁₅H₃₁C(O)— | 782.918 | 782.5 | |
| | | (SEQ ID NO. 5) | | | | |

TABLE 11
CXCR5 i2 loop compounds (amide on C-terminus)

| Comp. | | | | MS | MS | Mol. |
|---|---|---|---|---|---|---|
| # | Loop | Sequence | N terminus | Theoretical | Observed | Weight |
| 9 | i2 | AIVHAVHAYRHRRLLSI | C₁₅H₃₁C(O)— | 750.930 | 750.95 | 2249.791 |
| | | (SEQ ID NO. 15) | | | | |
| 10 | i2 | IVHAVHAYRHRR | C₁₅H₃₁C(O)— | 585.054 | 585.15 | 1752.163 |
| | | (SEQ ID NO. 16) | | | | |
| 11 | i2 | VHAVHAYRHRR | C₁₅H₃₁C(O)— | 547.335 | 547.4 | 1639.005 |
| | | (SEQ ID NO. 17) | | | | |
| 12 | i2 | HAVHAYRHRR | C₁₅H₃₁C(O)— | 514.291 | 514.35 | 1539.874 |
| | | (SEQ ID NO. 18) | | | | |
| 13 | i2 | HAVHAYRHRRLLSI | C₁₅H₃₁C(O)— | 656.475 | 656.5 | 1966.425 |
| | | (SEQ ID NO. 19) | | | | |
| 14 | i2 | HAVHAYRHRRLLS | C₁₅H₃₁C(O)— | 618.756 | 618.85 | 1853.267 |
| | | (SEQ ID NO. 20) | | | | |
| 15 | i2 | HAVHAYRHRRLL | C₁₅H₃₁C(O)— | 589.730 | 589.8 | 1766.19 |
| | | (SEQ ID NO. 21) | | | | |
| 16 | i2 | HAVHAYRHRRL | C₁₅H₃₁C(O)— | 552.011 | 552.1 | 1653.032 |
| | | (SEQ ID NO. 22) | | | | |
| 17 | i2 | HAYRHRRLLSI | C₁₅H₃₁C(O)— | 554.025 | 553.7 | 1659.076 |
| | | (SEQ ID NO. 26) | | | | |
| 18 | i2 | VHAYRHRRLLSI | C₁₅H₃₁C(O)— | 587.069 | 587 | 1758.207 |
| | | (SEQ ID NO. 27) | | | | |
| 19 | i2 | AVHAYRHRRLLSI | C₁₅H₃₁C(O)— | 610.762 | 610.7 | 1829.285 |
| | | (SEQ ID NO. 28) | | | | |
| 20 | i2 | AAVHAYRHRRLLSI | C₁₅H₃₁C(O)— | 634.454 | 634.2 | 1900.363 |
| | | (SEQ ID NO. 29) | | | | |
| 21 | i2 | HAAHAYRHRRLLSI | C₁₅H₃₁C(O)— | 647.124 | 646.9 | 1938.371 |
| | | (SEQ ID NO. 30) | | | | |
| 22 | i2 | IVHAVHAYRHRRLLSI | C₁₅H₃₁C(O)— | 727.238 | 727.2 | 2178.713 |
| | | (SEQ ID NO. 31) | | | | |
| 23 | i2 | VHAVHAYRHRRLLSI | C₁₅H₃₁C(O)— | 689.519 | | 2065.556 |
| | | (SEQ ID NO. 32) | | | | |
| 24 | i2 | hAVHAYRHRRLLSI | C₁₅H₃₁C(O)— | 656.475 | 656.2 | 1966.425 |
| | | (SEQ ID NO. 33) | | | | |
| 25 | i2 | HaVHAYRHRRLLSI | C₁₅H₃₁C(O)— | 656.077 | 656.4 | 1729 |
| | | (SEQ ID NO 34) | | | | |
| 26 | i2 | HAHhAYRHRRLLSI | C₁₅H₃₁C(O)— | 668.740 | 669 | 1867.293 |
| | | (SEQ ID NO. 35) | | | | |
| 27 | i2 | HAvHAYRHRRLLS | C₁₅H₃₁C(O)— | 618.756 | 618.6 | 1853.267 |
| | | (SEQ ID NO. 36) | | | | |
| 28 | i2 | HAVAAYRHRRLLSI | C₁₅H₃₁C(O)— | 634.454 | 634.45 | 1900.363 |
| | | (SEQ ID NO. 37) | | | | |
| 29 | i2 | HAVHAARHRRLLSI | C₁₅H₃₁C(O)— | 625.776 | 626.2 | 1874.329 |
| | | (SEQ ID NO. 38) | | | | |
| 30 | i2 | HAVHAYAHRRLLSI | C₁₅H₃₁C(O)— | 628.106 | 627.9 | 1881.317 |
| | | (SEQ ID NO. 39) | | | | |
| 31 | i2 | HAVHAYRARRLLSI | C₁₅H₃₁C(O)— | 951.182 | 951.65 | 1900.363 |
| | | (SEQ ID NO. 40) | | | | |
| 32 | i2 | HAVHAYRHARLLSI | C₁₅H₃₁C(O)— | 628.106 | 628.4 | 1881.317 |
| | | (SEQ ID NO. 41) | | | | |
| 33 | i2 | HAVHAYRHRALLSI | C₁₅H₃₁C(O)— | 628.106 | 628.05 | 1881.317 |
| | | (SEQ ID NO. 42) | | | | |
| 34 | i2 | HAVHAYRHRRALSI | C₁₅H₃₁C(O)— | 642.448 | 642.45 | 1924.345 |
| | | (SEQ ID NO. 43) | | | | |
| 35 | i2 | HAVHAYRHRRLASI | C₁₅H₃₁C(O)— | 642.448 | 642.45 | 1924.345 |
| | | (SEQ ID NO. 44) | | | | |
| 36 | i2 | HAVHAYRHRRLLAI | C₁₅H₃₁C(O)— | 651.142 | 651.05 | 1950.425 |
| | | (SEQ ID NO. 45) | | | | |
| 37 | i2 | HAVHAYRHRRLLSA | C₁₅H₃₁C(O)— | 642.060 | 642.4 | 1940.344 |
| | | (SEQ ID NO. 46) | | | | |
| 38 | i2 | HAVhAYRHRRLLSI | C₁₅H₃₁C(O)— | 656.475 | 656.75 | 1966.425 |
| | | (SEQ ID NO. 47) | | | | |
| 39 | i2 | HAVHaYRHRRLLSI | C₁₅H₃₁C(O)— | 656.475 | 656.85 | 1966.425 |
| | | (SEQ ID NO. 48) | | | | |
| 40 | i2 | HAVHAyRHRRLLSI | C₁₅H₃₁C(O)— | 656.475 | 656.5 | 1729 |
| | | (SEQ ID NO. 49) | | | | |
| 41 | i2 | HAVHAYrHRRLLSI | C₁₅H₃₁C(O)— | 656.475 | 656.7 | 1966.425 |
| | | (SEQ ID NO. 50) | | | | |
| 42 | i2 | HAVHAYRhRRLLSI | C₁₅H₃₁C(O)— | 656.475 | 656.35 | 1966.425 |
| | | (SEQ ID NO. 51) | | | | |
| 43 | i2 | HAVHAYRHrRLLSI | C₁₅H₃₁C(O)— | 656.475 | 656.8 | 1966.425 |
| | | (SEQ ID NO. 52) | | | | |
| 44 | i2 | HAVHAYRHRrLLSI | C₁₅H₃₁C(O)— | 656.475 | 656.75 | 1966.425 |
| | | (SEQ ID NO. 53) | | | | |
| 45 | i2 | HAVHAYRHRRILSI | C₁₅H₃₁C(O)— | 656.475 | 656.75 | 1966.425 |
| | | (SEQ ID NO. 54) | | | | |
| 46 | i2 | HAVHAYRHRRLISI | C₁₅H₃₁C(O)— | 656.475 | 656.85 | 1966.425 |
| | | (SEQ ID NO. 55) | | | | |
| 47 | i2 | HAVHAYRHRRLLsI | C₁₅H₃₁C(O)— | 656.475 | 656.85 | 1966.425 |
| | | (SEQ ID NO. 56) | | | | |
| 48 | i2 | HAVHAYRHRRLLFI | C₁₅H₃₁C(O)— | 676.507 | 676.3 | 2026.521 |
| | | (SEQ ID NO. 57) | | | | |
| 49 | i2 | HAVHAYRHRRLLVI | C₁₅H₃₁C(O)— | 660.493 | 660.2 | 1978.478 |
| | | (SEQ ID NO. ) | | | | |
| 50 | i2 | HAVHAYRRRRLLS | C₁₅H₃₁C(O)— | 625.104 | 624.8 | 1872.313 |
| | | (SEQ ID NO. 60) | | | | |
| 51 | i2 | HAVHAYRRRRLLSI | C₁₅H₃₁C(O)— | 662.824 | 662.8 | 1985.471 |
| | | (SEQ ID NO. 60) | | | | |
| 52 | i2 | HAVHAYRHRRLLS | C₁₅H₃₁OC(O)16- | 624.089 | 624 | 1869.266 |
| | | (SEQ ID NO. 20) | Hydroxy-Pal | | | |
| 53 | i2 | HAVHAYRHRRLLS | C₁₁H₂₃OC₃H₆(CO)3- | 619.413 | 619.2 | 1903.347 |
| | | (SEQ ID NO. 20) | (dodecyloxy) | | | |
| | | | propanoate | | | |
| 54 | i2 | HAVHAYRHRRLLS | C₁₉H₃₈NOC(O)-2- | 647.124 | 646.8 | 1855.24 |
| | | (SEQ ID NO. 20) | acetamidoocta | | | |
| | | | decanoic acid | | | |
| 55 | i2 | HAVHAYRHRRLLS | C₁₇H₃₃C(O)- | 627.435 | 628 | 1938.371 |
| | | (SEQ ID NO. 20) | Elaidic | | | |
| 56 | i2 | HAVHAYRHRRLLS | C₁₇H₃₃C(O)- | 627.435 | 628 | 1879.304 |
| | | (SEQ ID NO. 20) | Oleic | | | |

TABLE 12
CXCR5 i3 loop compounds (amide on C-terminus)

| Comp. | | | | MS | MS | Mol. |
|---|---|---|---|---|---|---|
| # | Loop | Sequence | N terminus | Theoretical | Observed | Weight |
| 57 | i3 | RQAQRRPQRQK | C₁₅H₃₁C(O)— | 1689.080 | 1689.53 | 1689.064 |
| | | (SEQ ID NO. 62) | | | | |
| 58 | i3 | RQAQRRPQRQKAVRVAI | C₁₅H₃₁C(O)— | 2298.480 | 2298.82 | 2298.825 |
| | | (SEQ ID NO. 63) | | | | |
| 59 | i3 | RLRQAQRRPQRQK | C₁₅H₃₁C(O)— | 1958.260 | 1958.63 | 1958.408 |
| | | (SEQ ID NO. 64) | | | | |
| 60 | i3 | RQAQRRPQRQKAV | C₁₅H₃₁C(O)— | 1859.190 | 1859.63 | 1859.273 |
| | | (SEQ ID NO. 65) | | | | |
| 61 | i3 | GVVHRLRQAQRRPQRQK | C₁₅H₃₁C(O)— | 2350.480 | 2350.26 | 2350.86 |
| | | (SEQ ID NO. 224) | | | | |
| 62 | i3 | GVVHRLRQAQRRPQRQKAVRVAI | C₁₅H₃₁C(O)— | 987.874 | 987.8 | 2960.622 |
| | | (SEQ ID NO. 66) | | | | |
| 63 | i3 | VHRLRQAQRRPQRQK | C₁₅H₃₁C(O)— | 2194.390 | 2194.46 | 2194.678 |
| | | (SEQ ID NO. 68) | | | | |
| 64 | i3 | RLRQAQRRPQRQKAV | C₁₅H₃₁C(O)— | 2128.370 | 2128.47 | 2128.617 |
| | | (SEQ ID NO. 69) | | | | |
| 65 | i3 | RQAQRRPQRQKAVRV | C₁₅H₃₁C(O)— | 2114.350 | 2114.37 | 2114.59 |
| | | (SEQ ID NO. 72) | | | | |
| 66 | i3 | RRPQRQKAVRVAI | C₁₅H₃₁C(O)— | 908.652 | 908.7 | 1815.303 |
| | | (SEQ ID NO. 73) | | | | |
| 67 | i3 | RQAQRRPQRQKAVR | C₁₅H₃₁C(O)— | 672.820 | 673.1 | 2015.459 |
| | | (SEQ ID NO. 76) | | | | |
| 68 | i3 | RQAQRRPQRQKAVRVA | C₁₅H₃₁C(O)— | 729.556 | 729.6 | 2185.668 |
| | | (SEQ ID NO. 65) | | | | |
| 69 | i3 | RLRNANRRPNRNKAVRV | C₁₅H₃₁C(O)— | 776.942 | 776.9 | 2327.827 |
| | | (SEQ ID NO. 72) | | | | |
| 70 | i3 | RLRQAQRRPQR | C₁₅H₃₁C(O)— | 852.053 | 852.2 | 1702.106 |
| | | (SEQ ID NO. 79) | | | | |
| 71 | i3 | RLRQAQRRPQRQ | C₁₅H₃₁C(O)— | 916.118 | 916.1 | 1830.235 |
| | | (SEQ ID NO. 80) | | | | |
| 72 | i3 | RLRQAQRRPQRQKA | C₁₅H₃₁C(O)— | 1015.743 | 1015.9 | 2029.485 |
| | | (SEQ ID NO. 81) | | | | |
| 73 | i3 | RLRQAQRRPQRQKAVR | C₁₅H₃₁C(O)— | 762.601 | 762.2 | 2284.802 |
| | | (SEQ ID NO. 82) | | | | |
| 74 | i3 | RLRQAQRRPQRQKAVRV | C₁₅H₃₁C(O)— | 795.644 | 795.4 | 2383.933 |
| | | (SEQ ID NO. 82) | | | | |
| 75 | i3 | RLRTATRRPTRTKAVRV | C₁₅H₃₁C(O)— | 569.958 | 570.25 | 2275.832 |
| | | (SEQ ID NO. 83) | | | | |
| 76 | i3 | GRLRQAQRRPQRQK | C₁₅H₃₁C(O)— | 672.820 | 672.15 | 2015.459 |
| | | (SEQ ID NO. 84) | | | | |
| 77 | i3 | HRLRQAQRRPQR | C₁₅H₃₁C(O)— | 614.082 | 614 | 1839.245 |
| | | (SEQ ID NO. 85) | | | | |
| 78 | i3 | HRLRQAQRRPQRQK | C₁₅H₃₁C(O)— | 699.516 | 699.35 | 2095.547 |
| | | (SEQ ID NO. 86) | | | | |
| 79 | i3 | GSGRLRQAQRRPQRQKAVRV | C₁₅H₃₁C(O)— | 518.023 | 518.3 | 2585.113 |
| | | (SEQ ID NO. 87) | | | | |

TABLE 13
CXCR5 i4 loop compounds (amide on C-terminus)

| | | | MS | MS | Mol. |
|---|---|---|---|---|---|
| Comp. # | Sequence | N terminus | Theoretical | Observed | Weight |
| 80 | RSDLSRLLTKLGS | C₁₅H₃₁C(O)— | 842.544 | 842.5 | 1683.088 |
| | (SEQ ID NO. 89) | | | | |
| 81 | LLTKLGSTGPASL | C₁₅H₃₁C(O)— | 748.450 | 748.5 | 1494.9 |
| | (SEQ ID NO. 91) | | | | |
| 82 | LSRLLTKLGSTGP | C₁₅H₃₁C(O)— | 791.004 | 791 | 1580.008 |
| | (SEQ ID NO. 92) | | | | |
| 83 | VKFRSDLSRLLTKLGSTGPASLS | C₁₅H₃₁C(O)— | 1336.613 | 1336.4 | 2671.226 |
| | (SEQ ID NO. 93) | | | | |

TABLE 14
CXCR5 i1 loop compounds

| Comp. | |
|---|---|
| # | Structure |
| 1 | 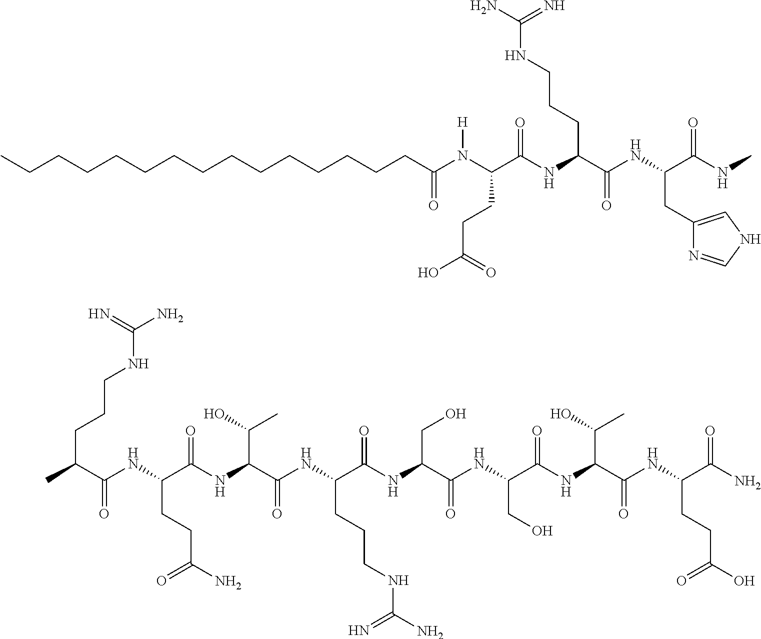 |
| 2 | 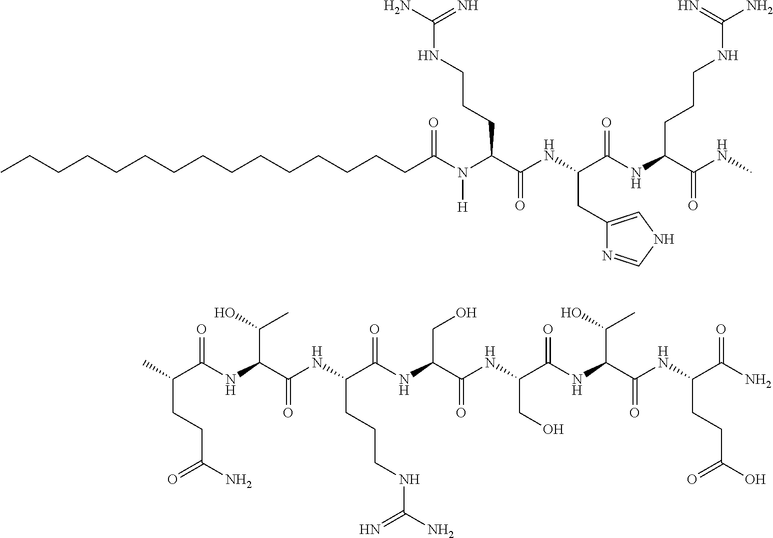 |
| 3 | 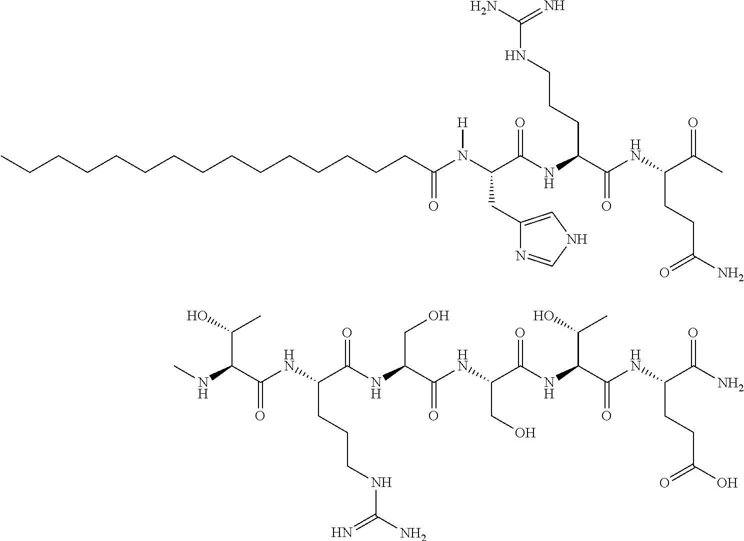 |
| 4 | 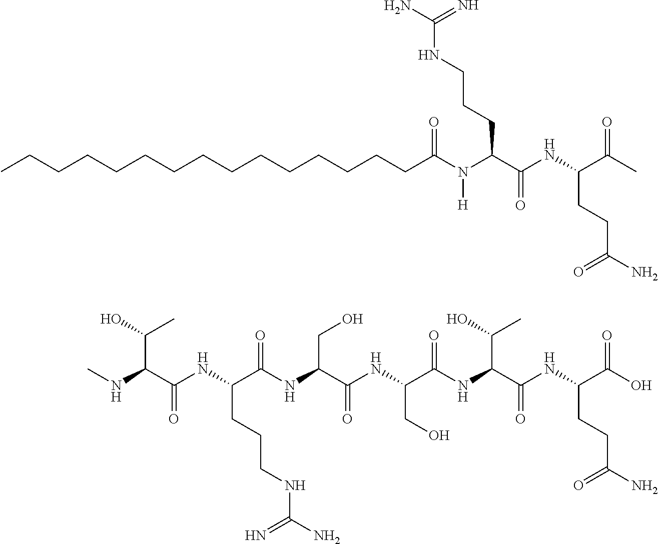 |
| 5 | 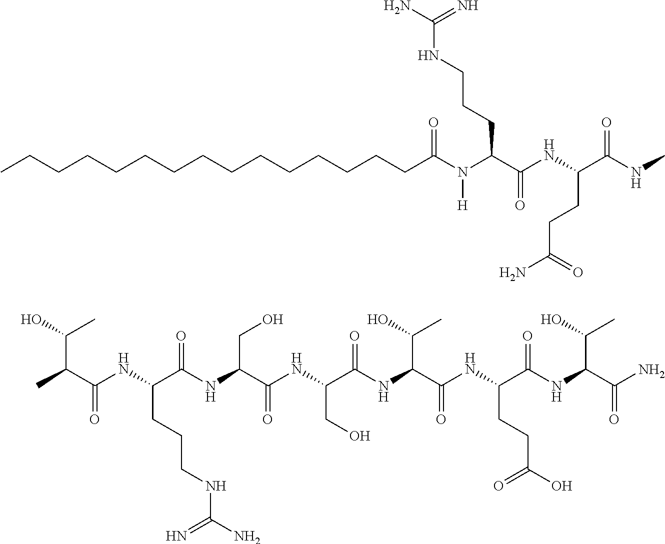 |
| 6 | 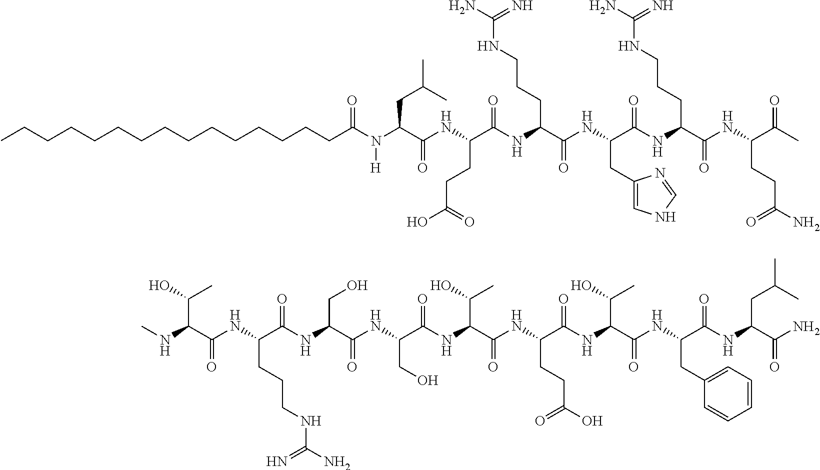 |
| 7 | 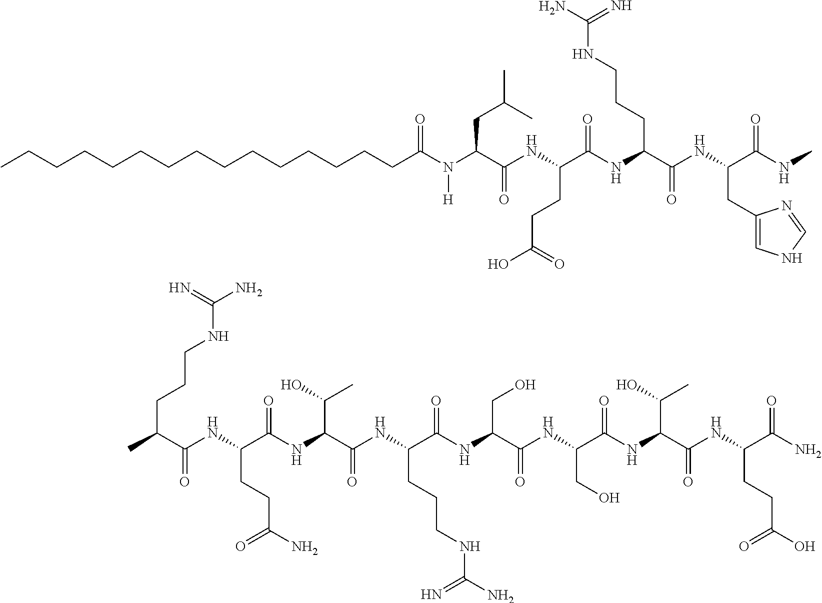 |
| 8 | 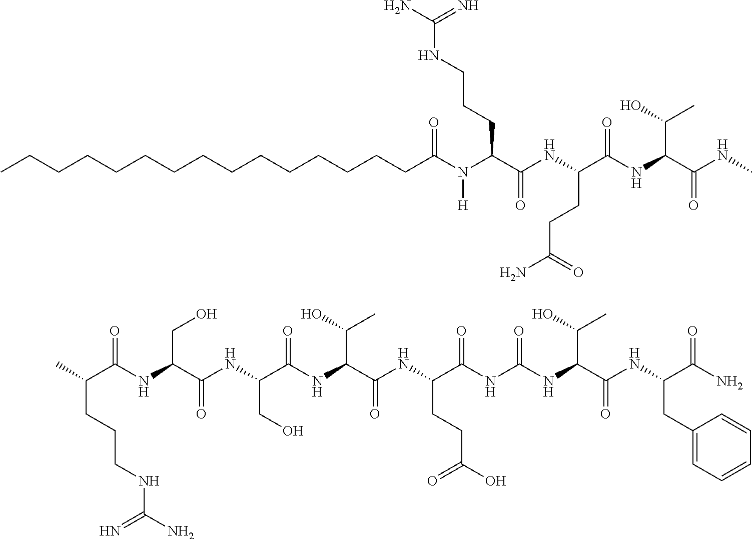 |
| 94 | 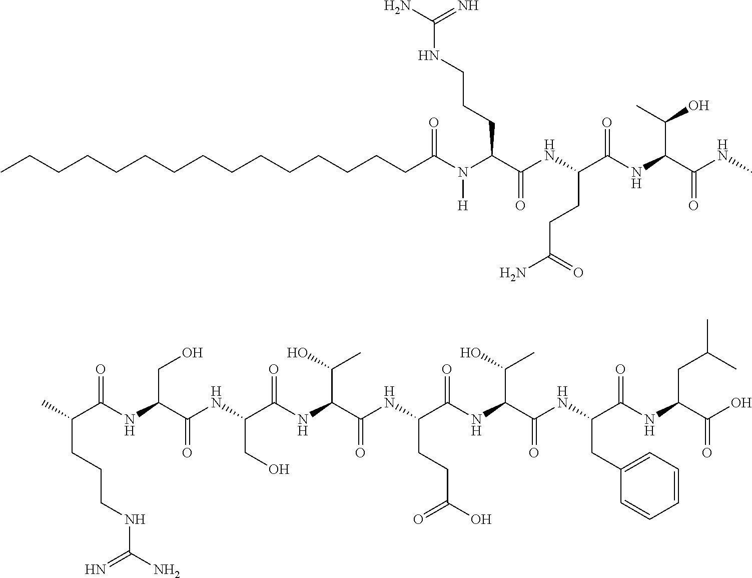 |

TABLE 15
CXCR5 i2 loop compounds

| Comp. | |
|---|---|
| # | Structure |
| 9 | 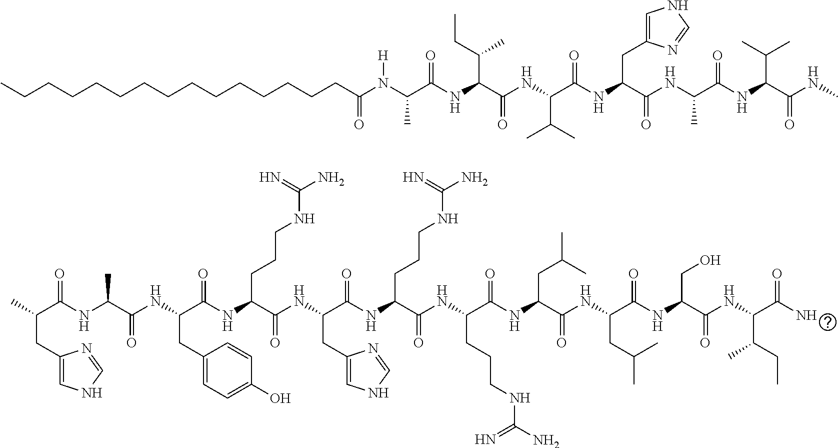 |
| 10 | 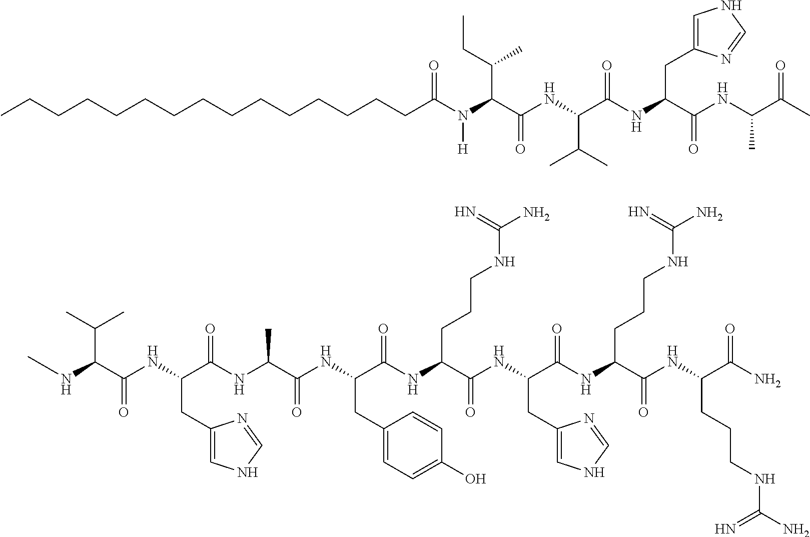 |
| 11 | 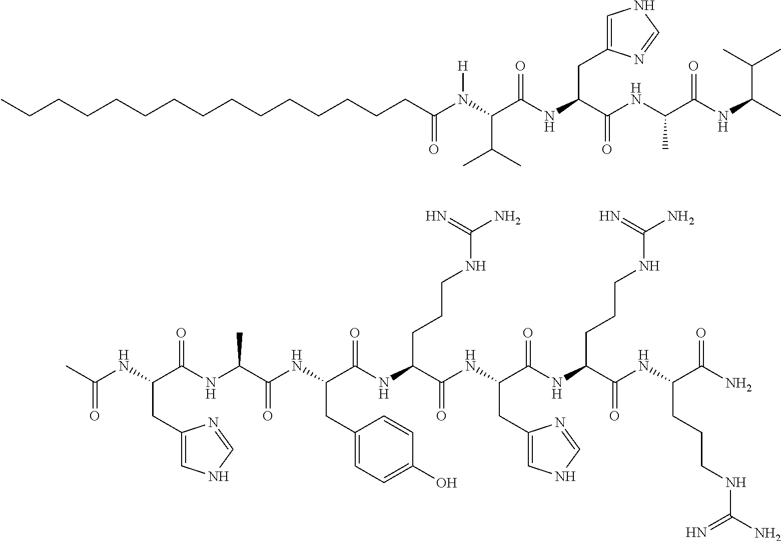 |
| 12 | 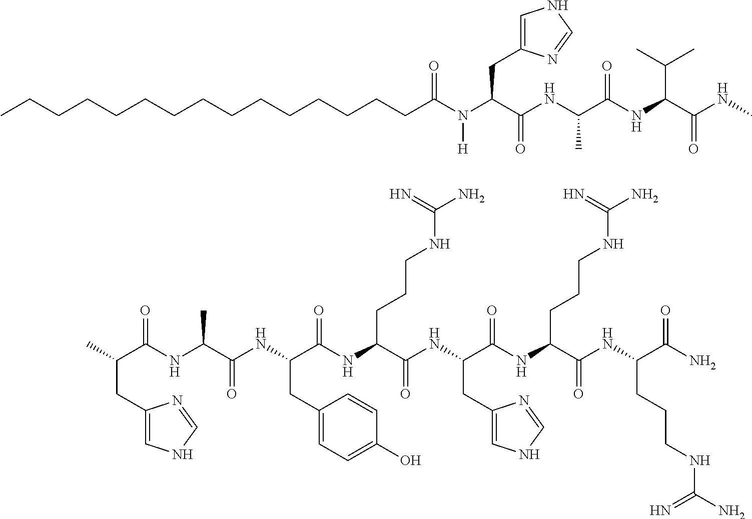 |
| 13 | 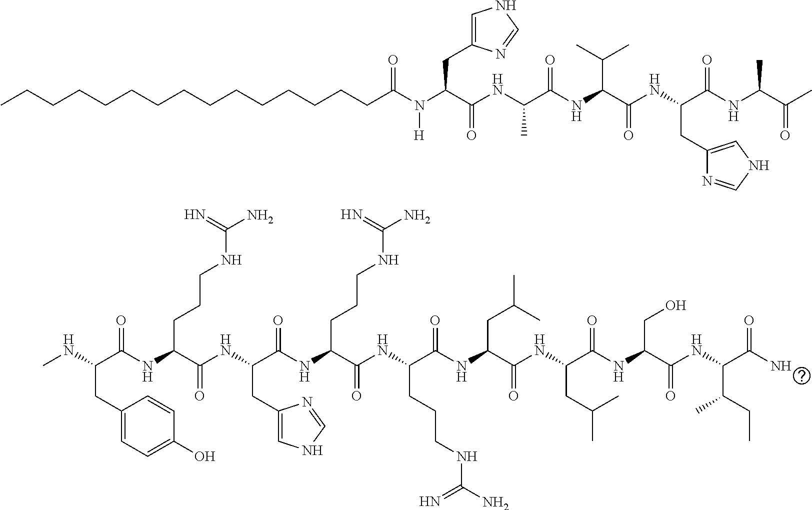 |
| 14 | 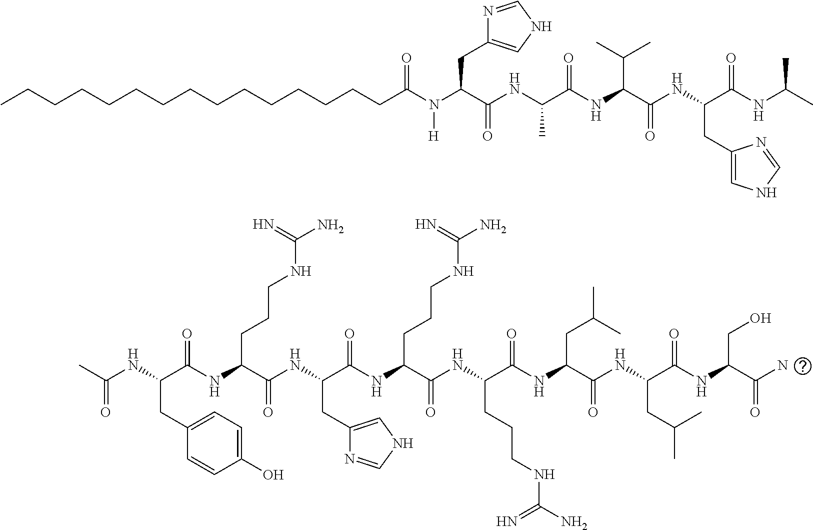 |
| 15 | 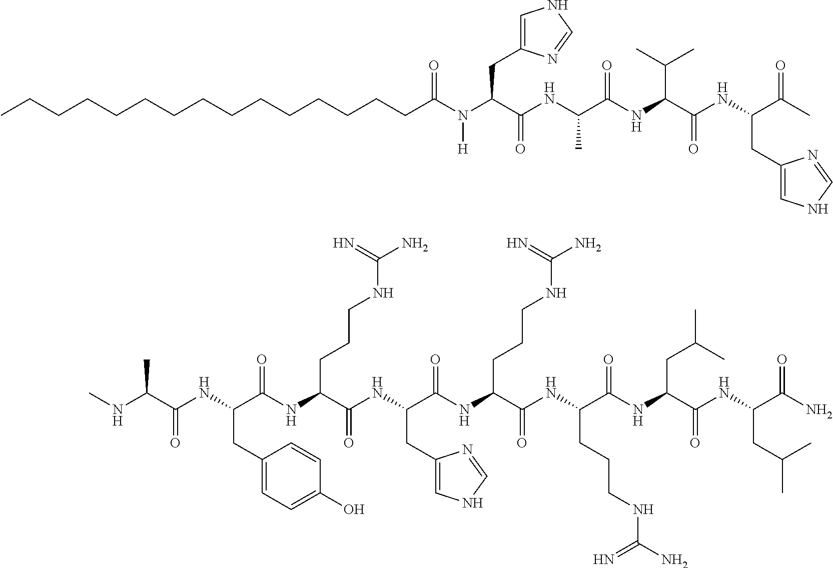 |
| 16 | 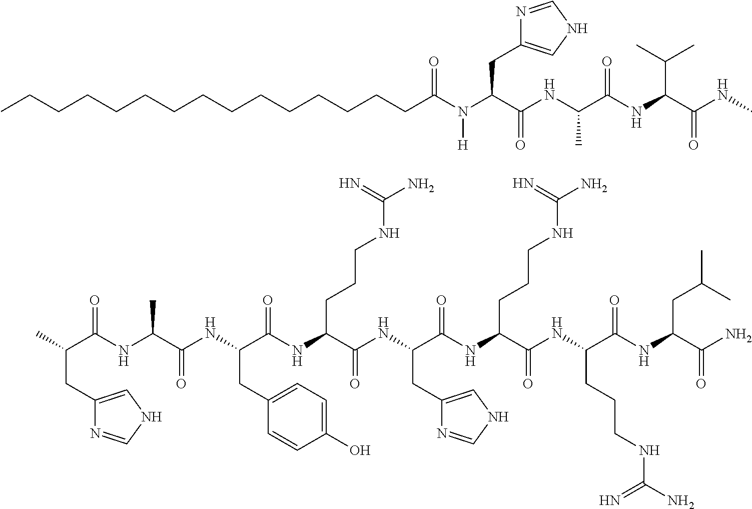 |
| 17 | 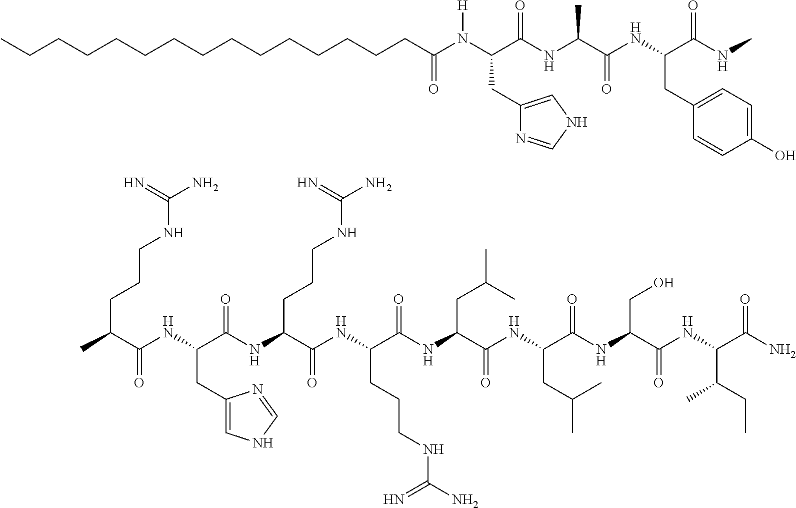 |
| 18 | 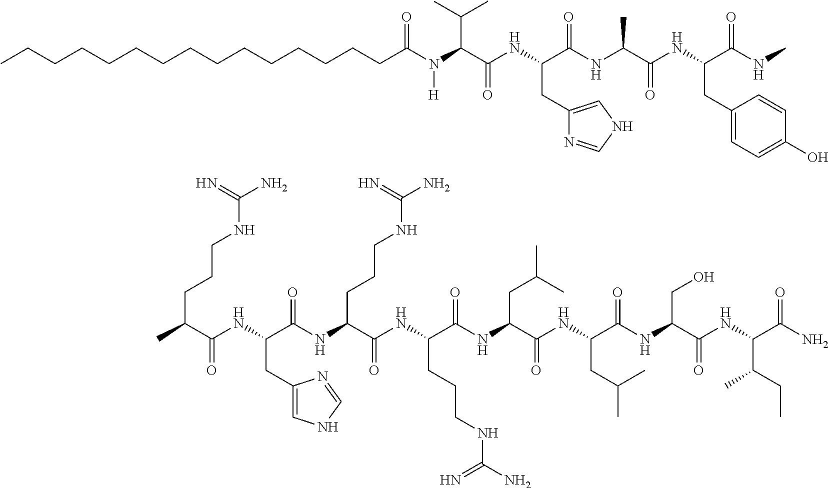 |
| 19 | 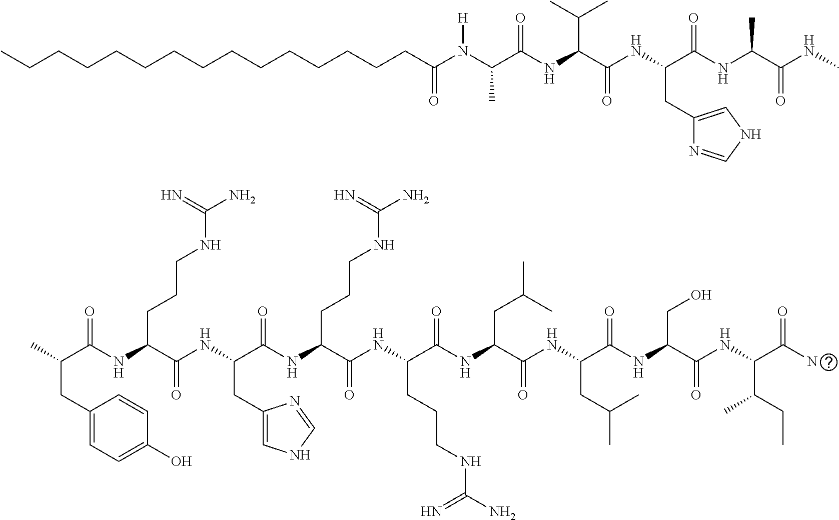 |
| 20 | 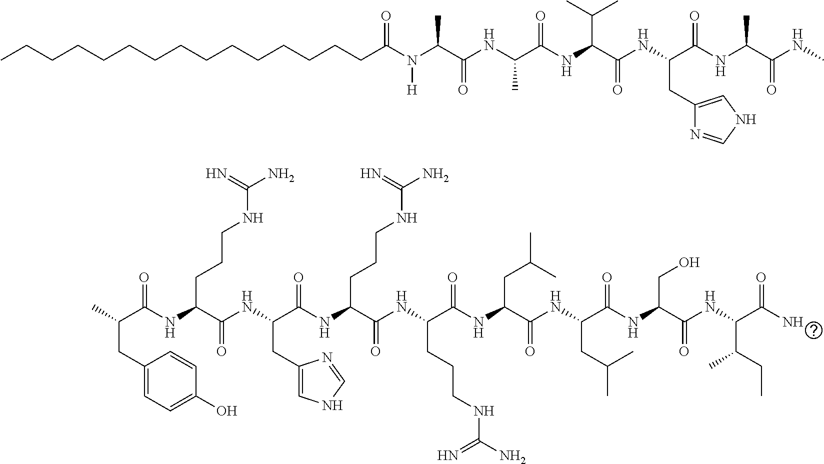 |
| 21 | 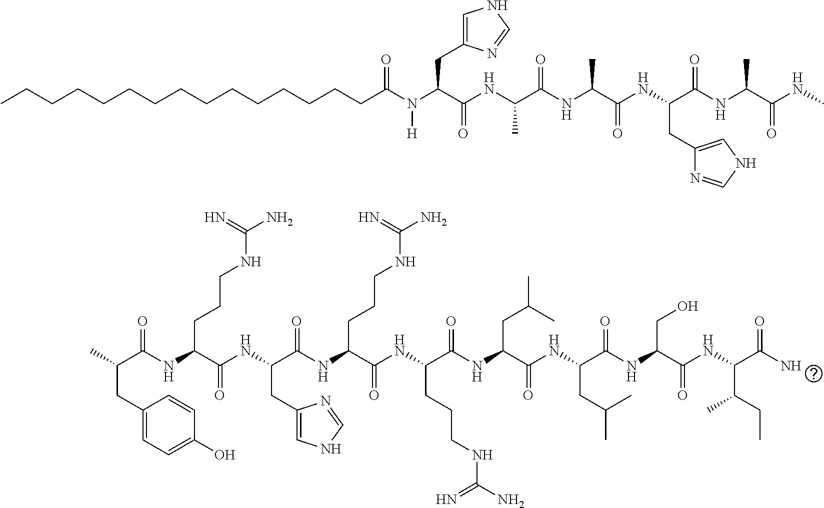 |
| 22 | 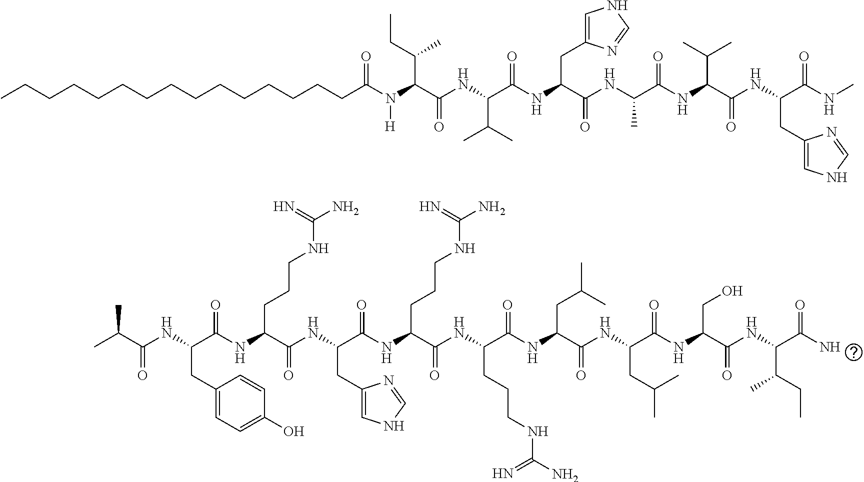 |
| 23 | 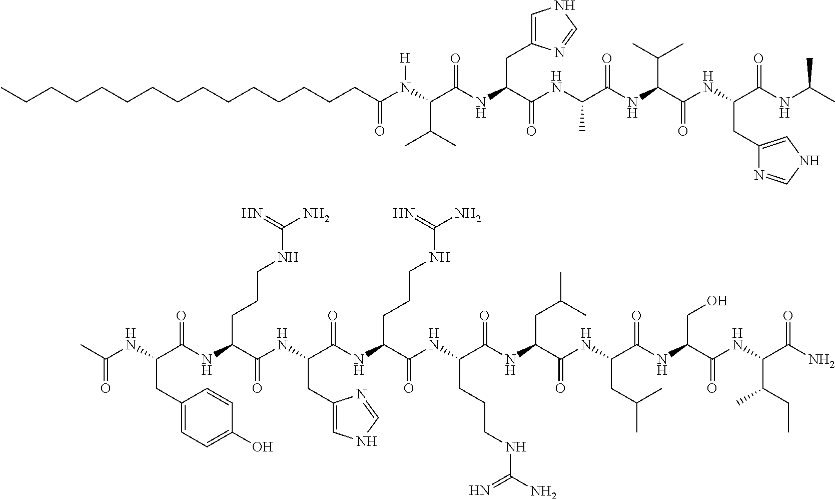 |
| 24 | 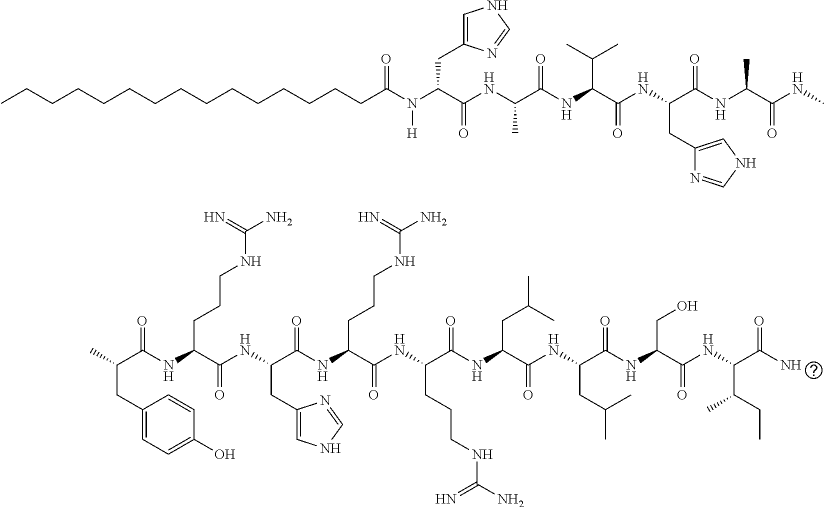 |
| 25 | 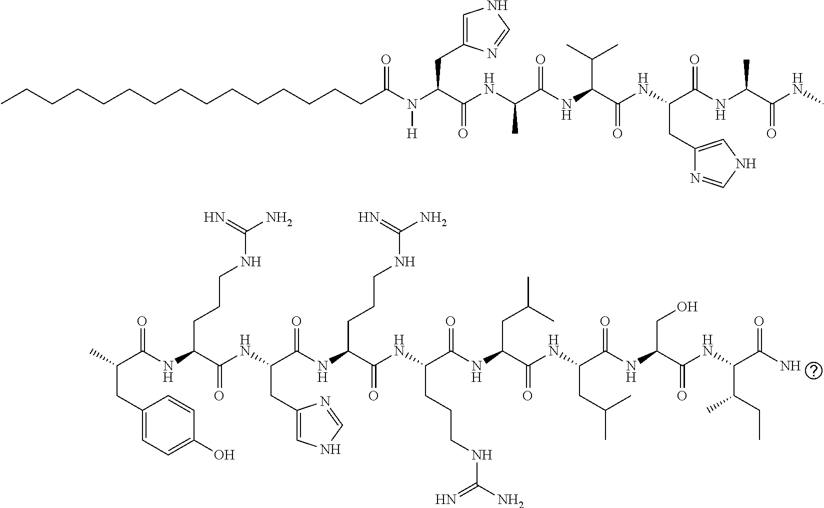 |
| 26 | 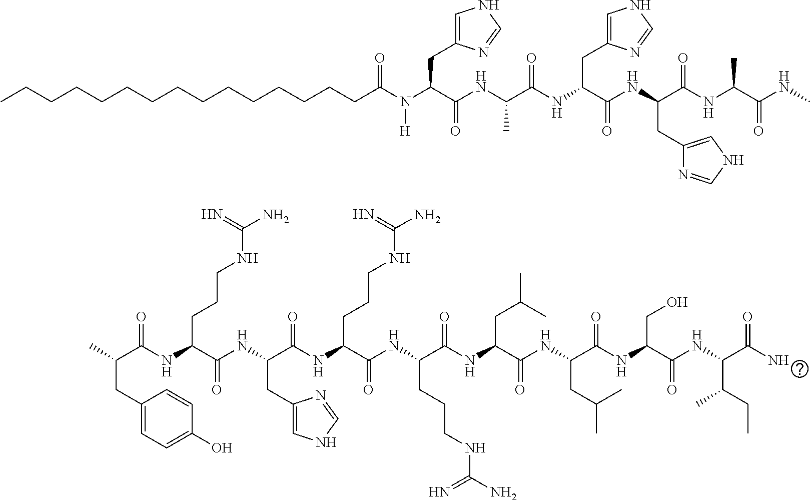 |
| 27 | 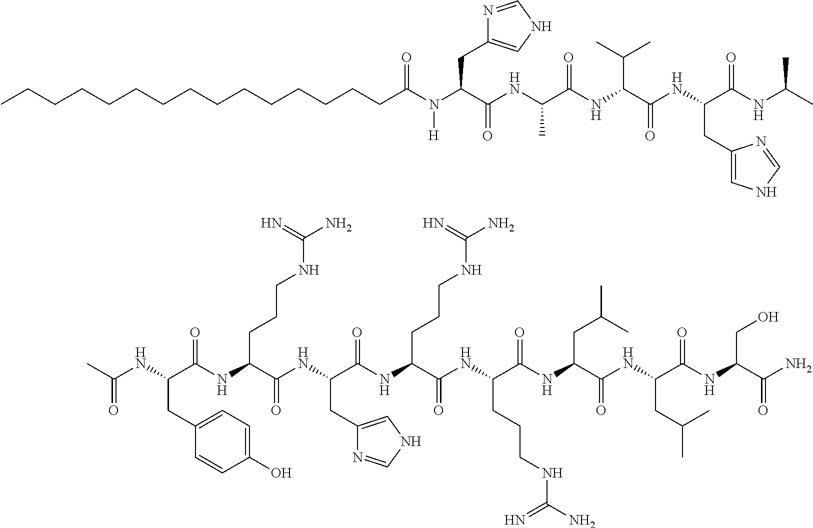 |
| 28 | 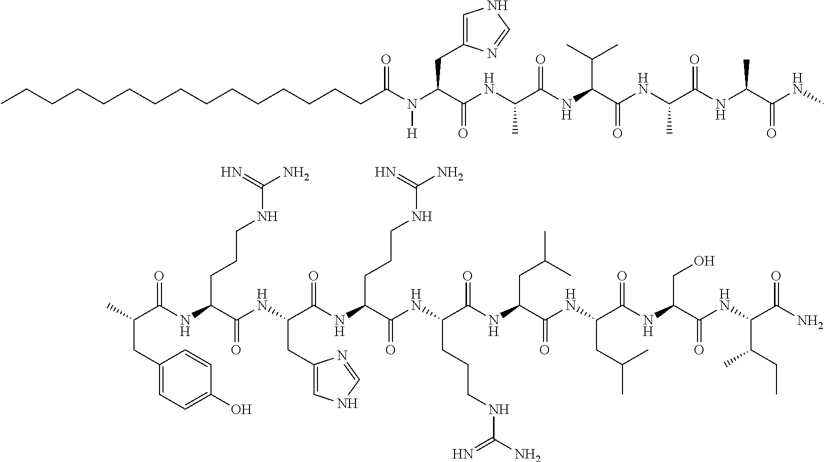 |
| 29 | 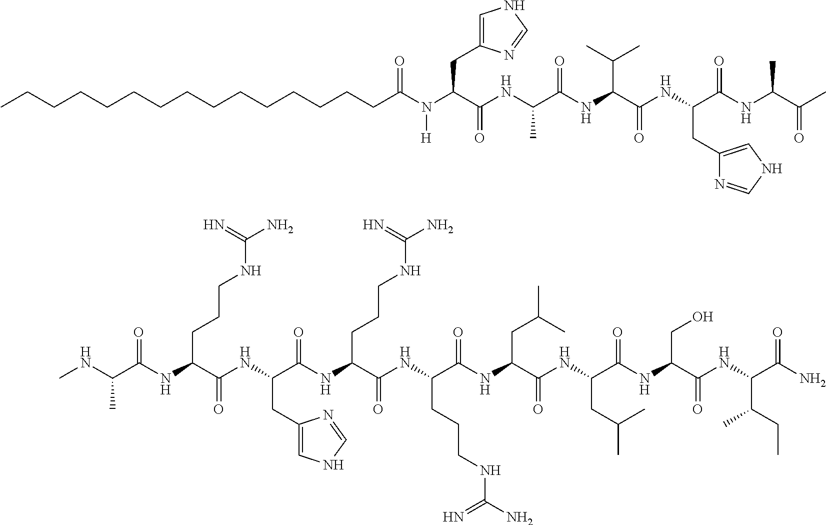 |
| 30 | 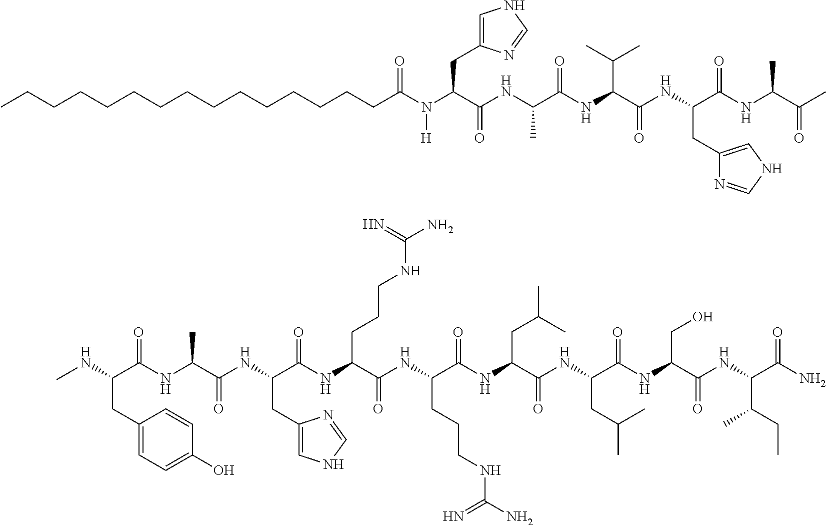 |
| 31 | 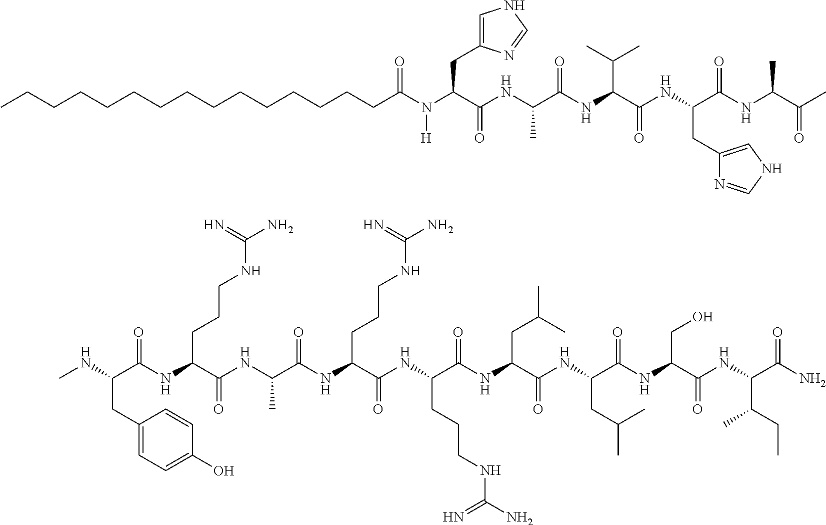 |
| 32 | 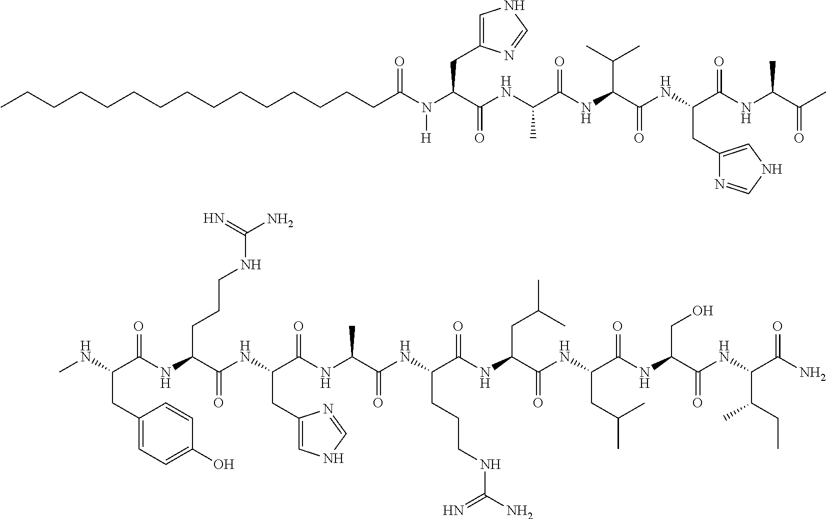 |
| 33 | 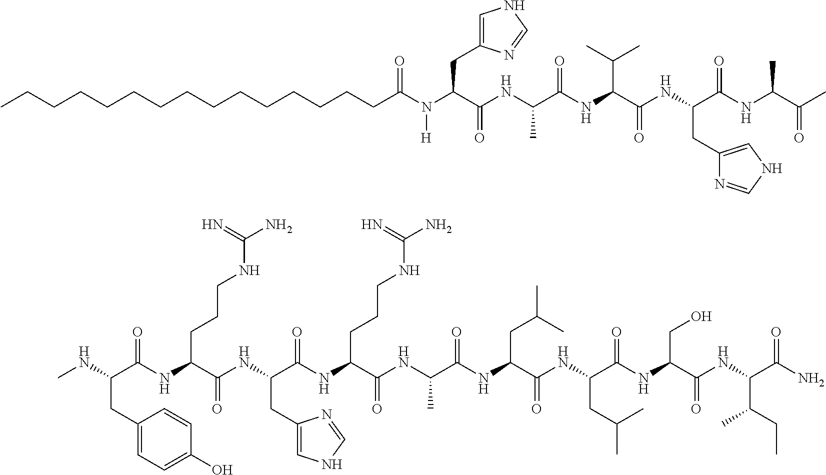 |
| 34 | 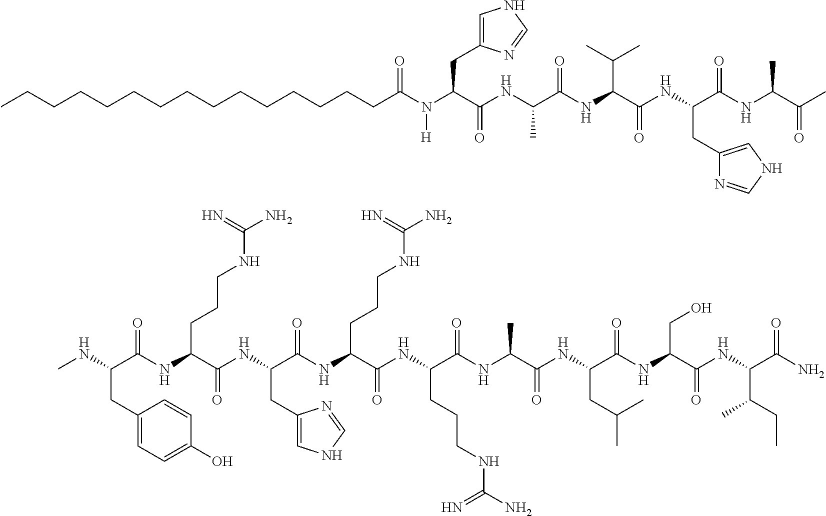 |
| 35 | 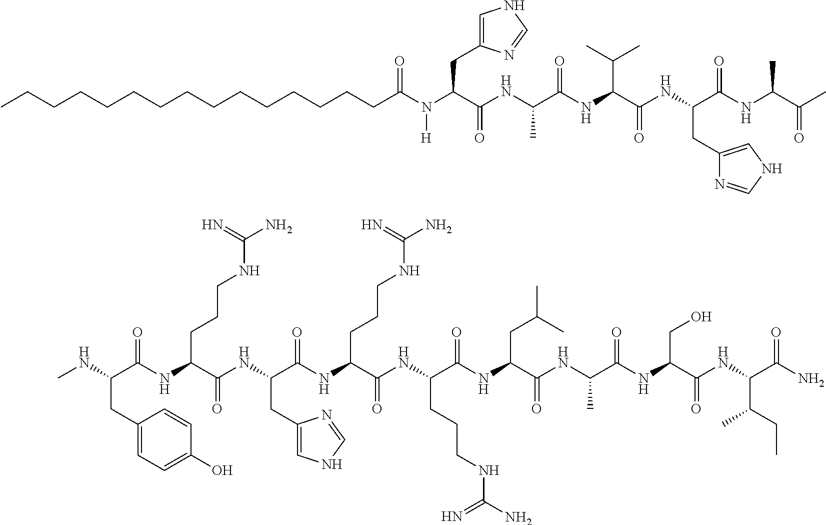 |
| 36 | 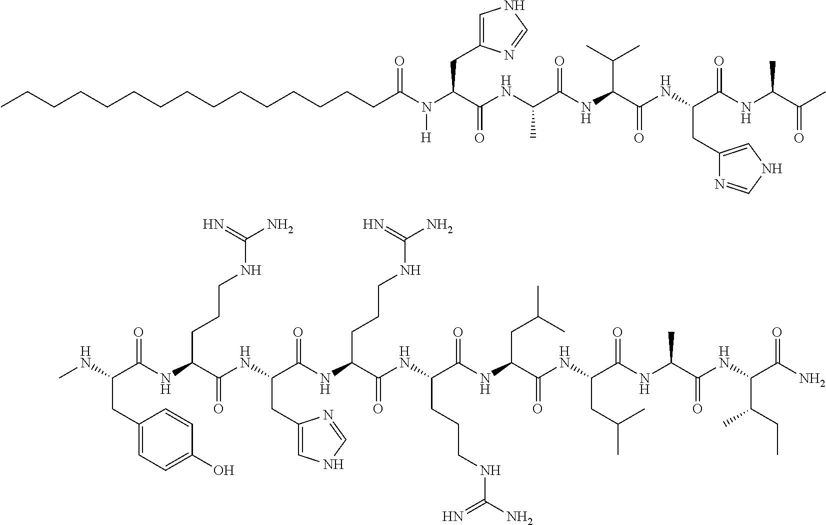 |
| 37 | 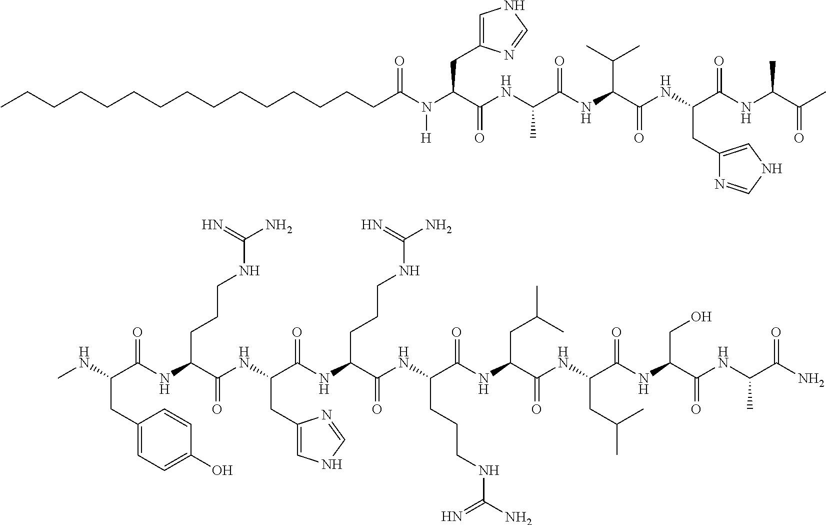 |
| 38 | 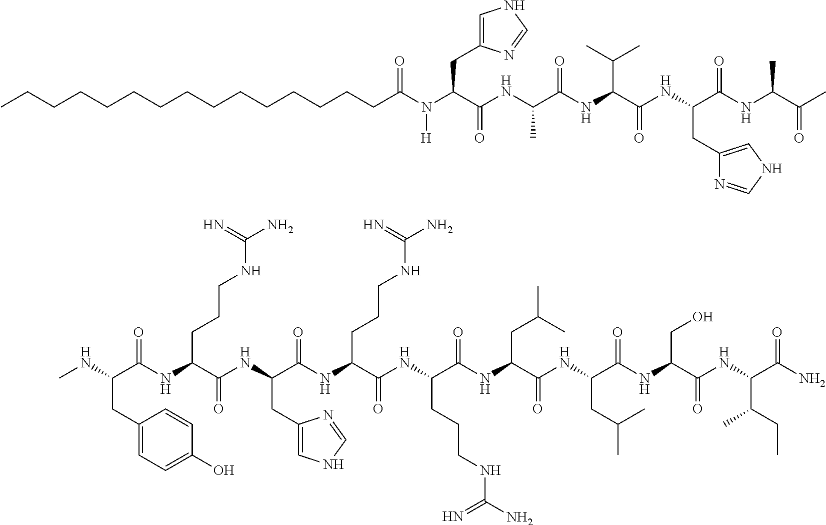 |
| 39 | 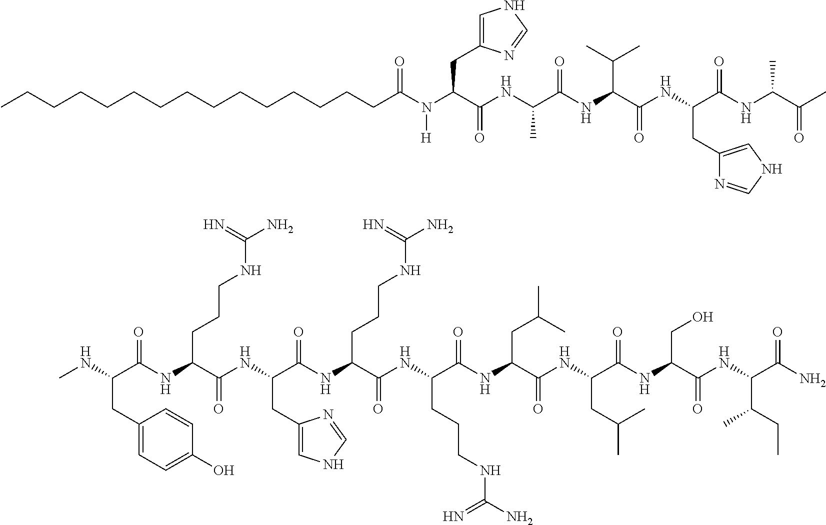 |
| 40 | 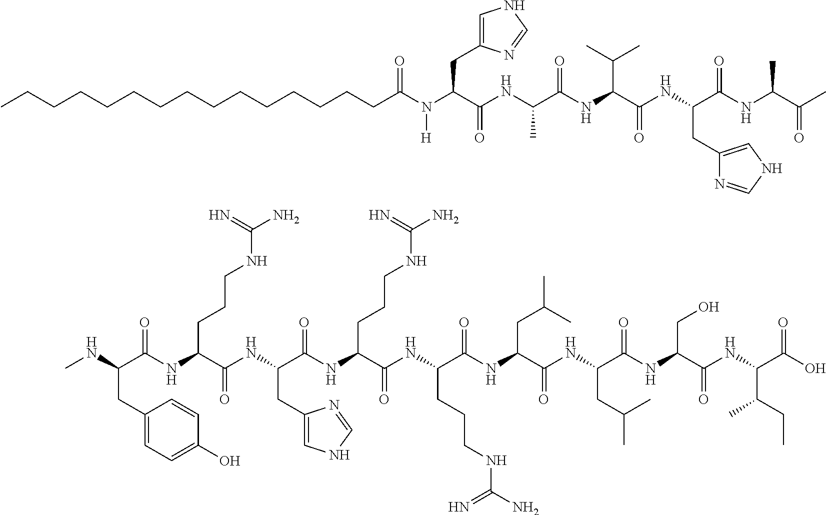 |
| 41 | 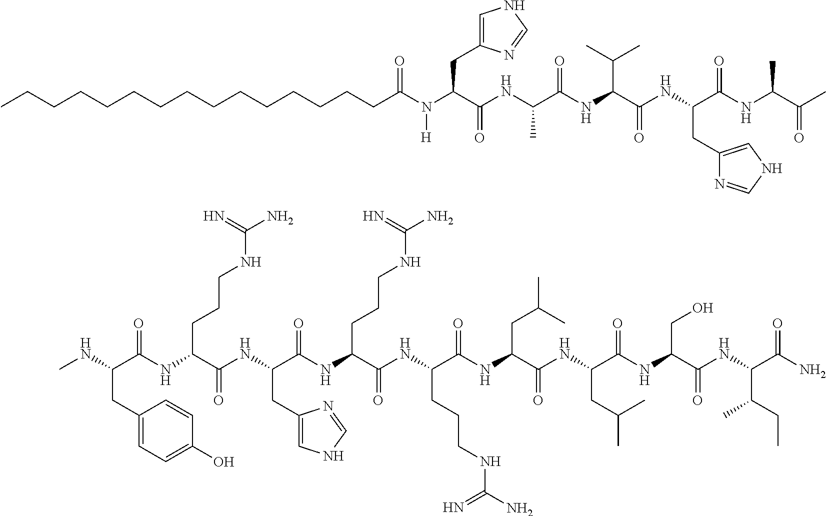 |
| 42 | 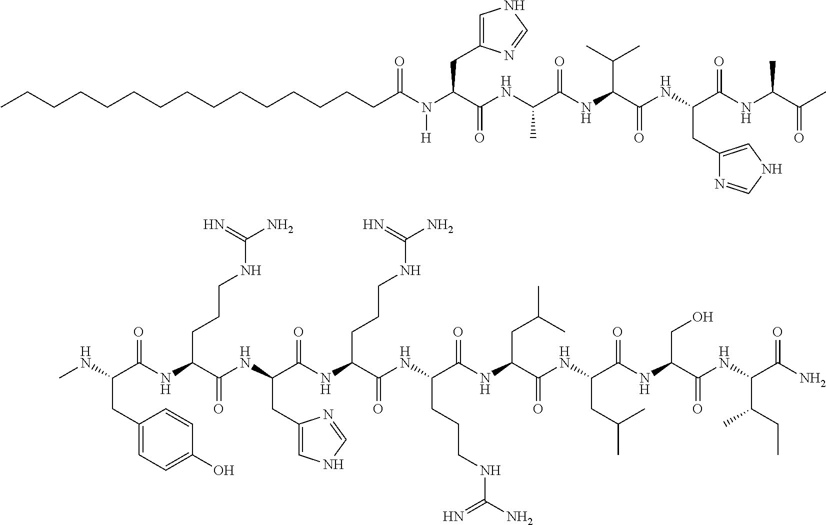 |
| 43 | 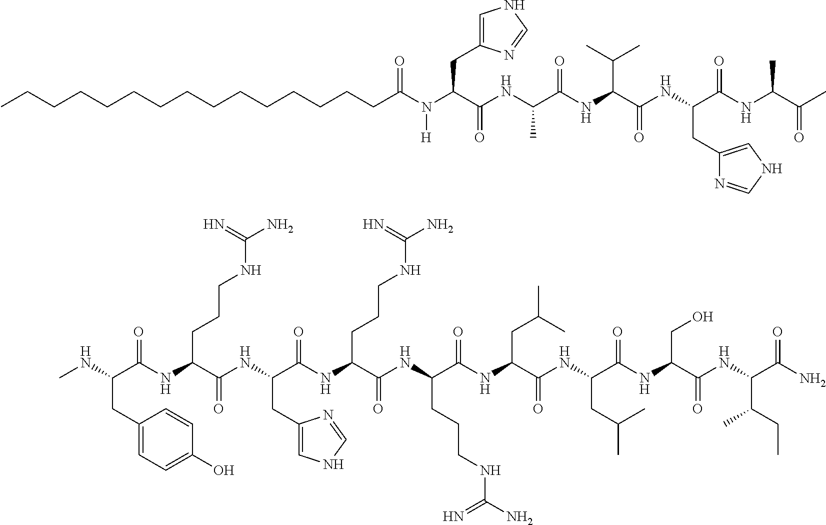 |
| 44 | 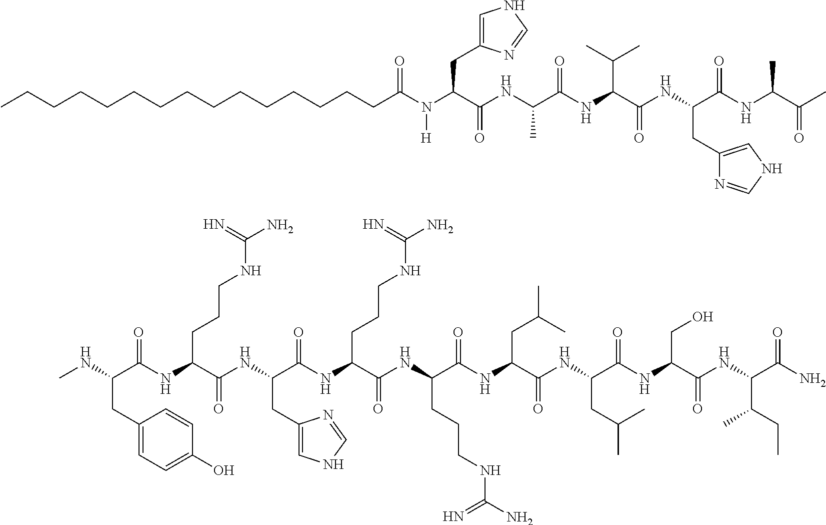 |
| 45 | 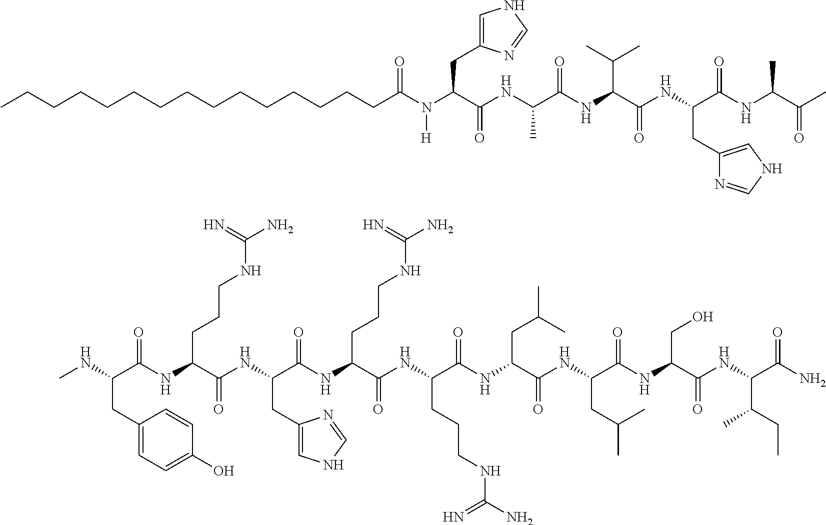 |
| 46 | 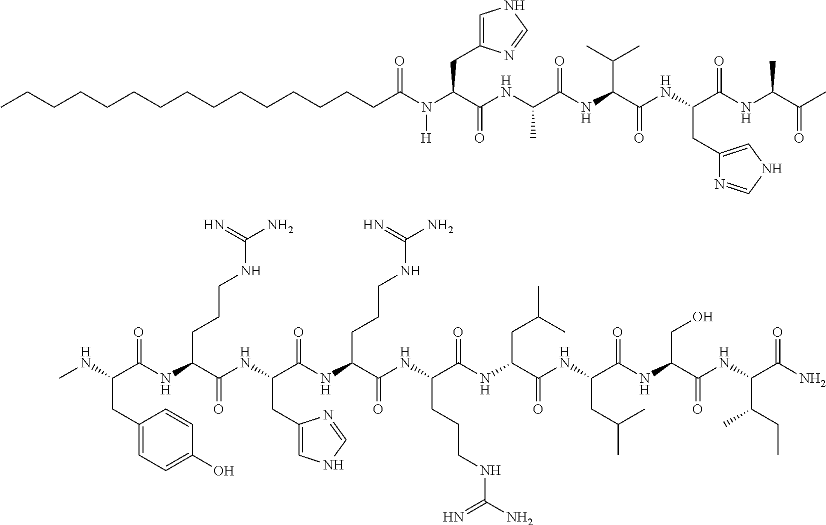 |
| 47 | 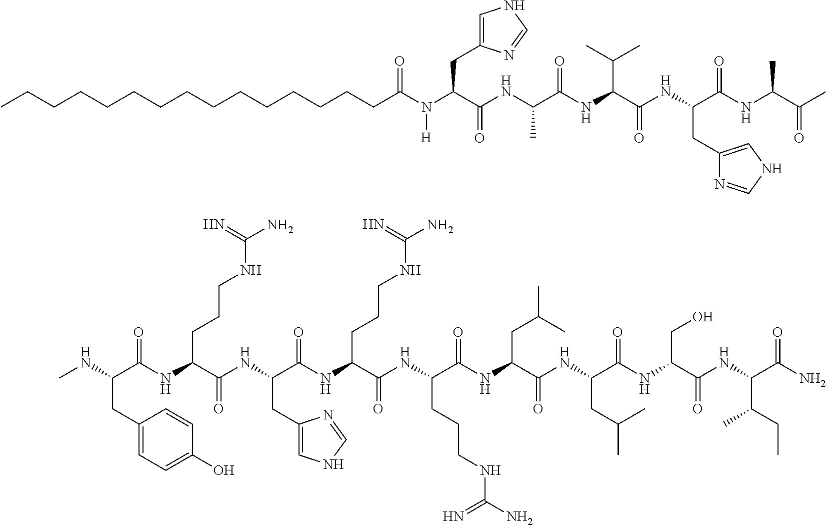 |
| 48 | 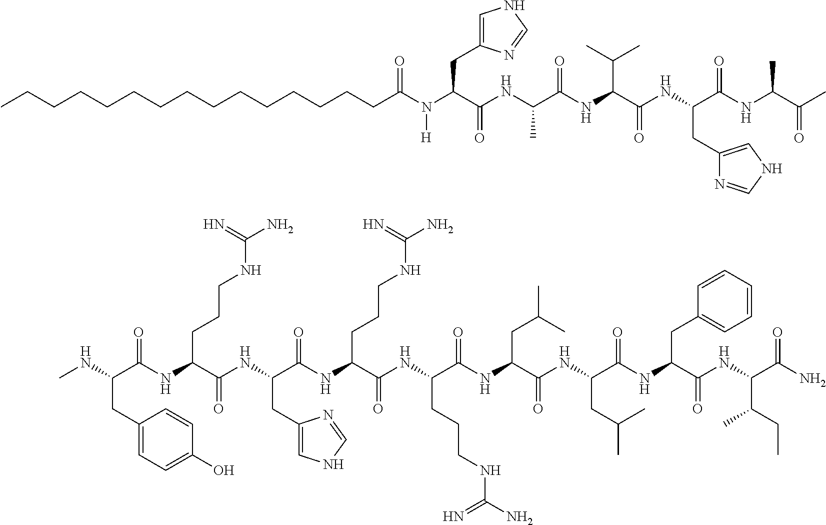 |
| 49 | 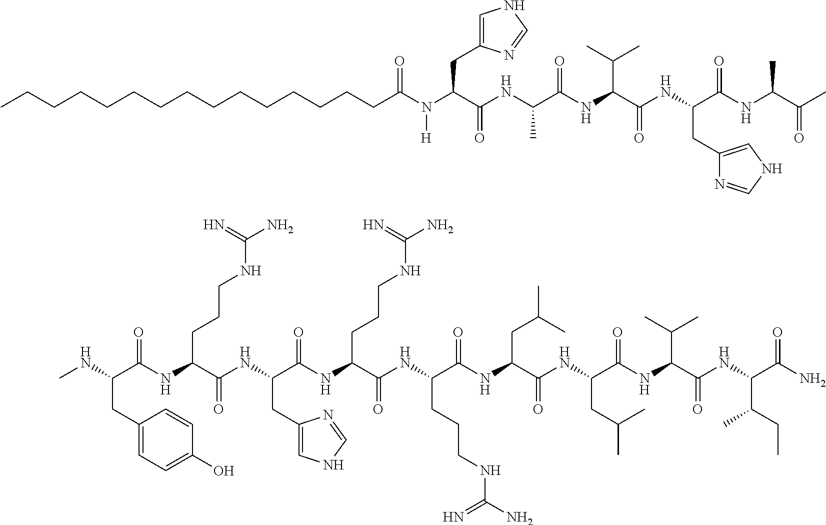 |
| 50 | 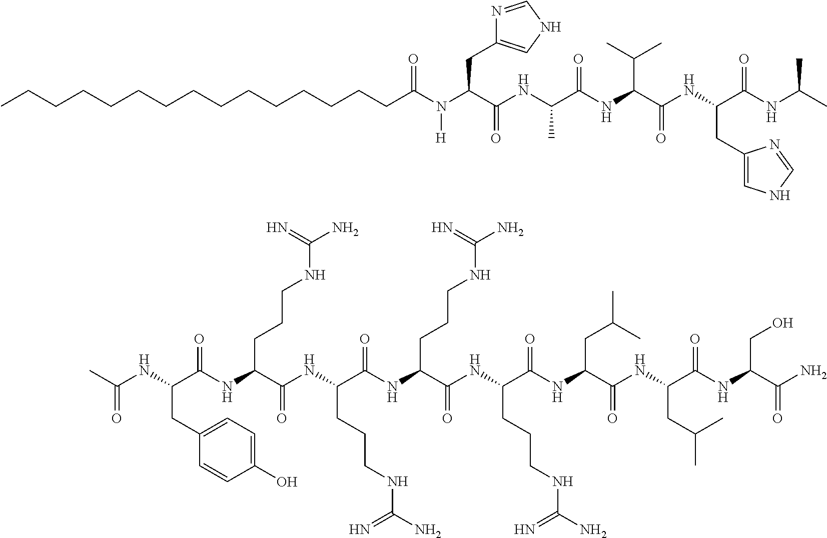 |
| 51 | 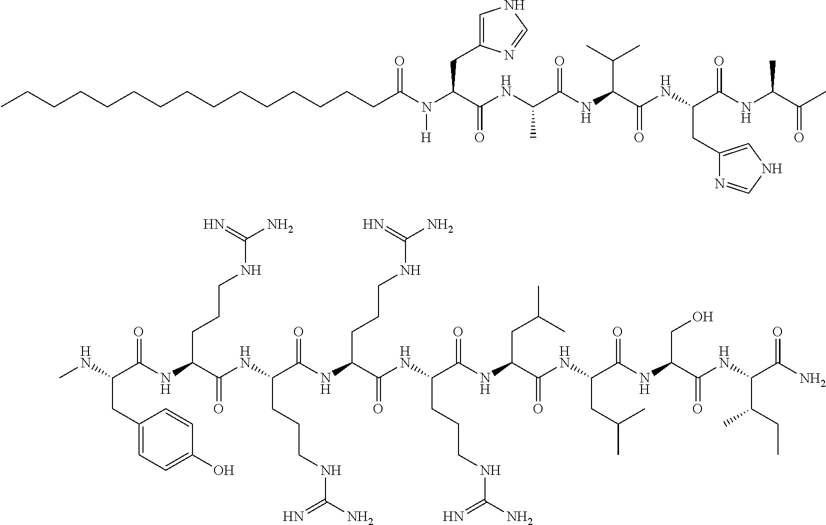 |
| 52 | 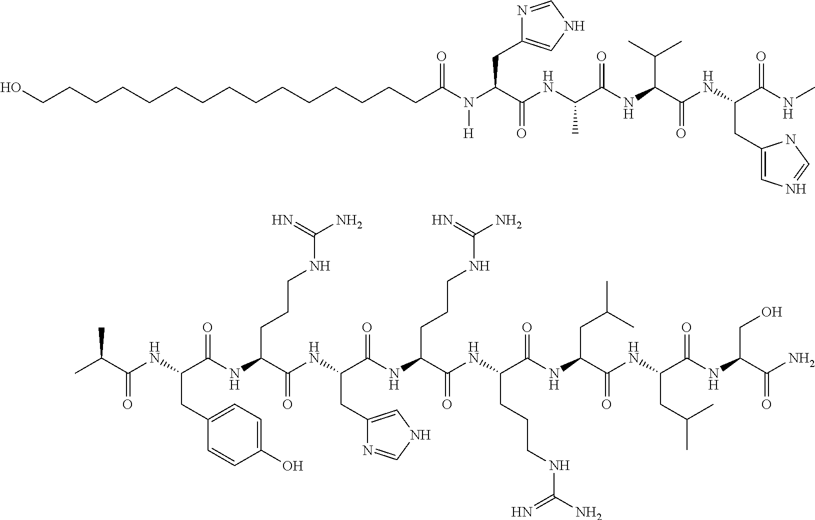 |
| 53 | 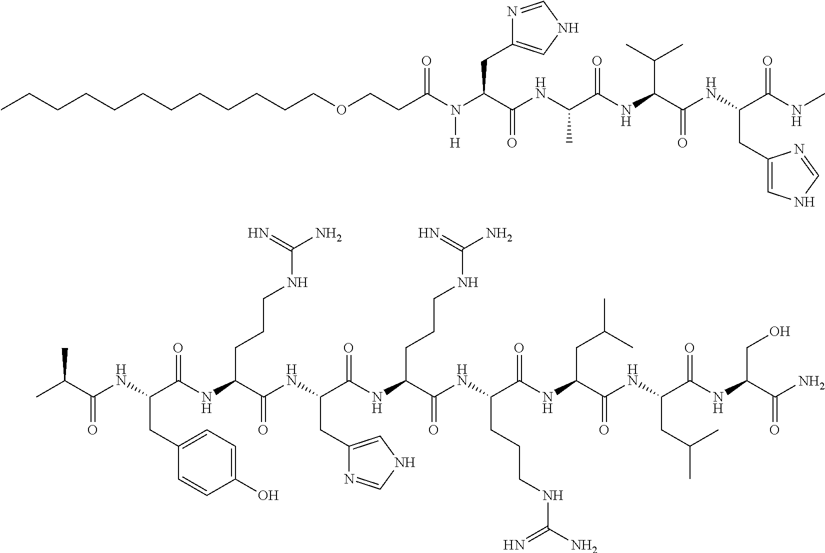 |
| 54 | 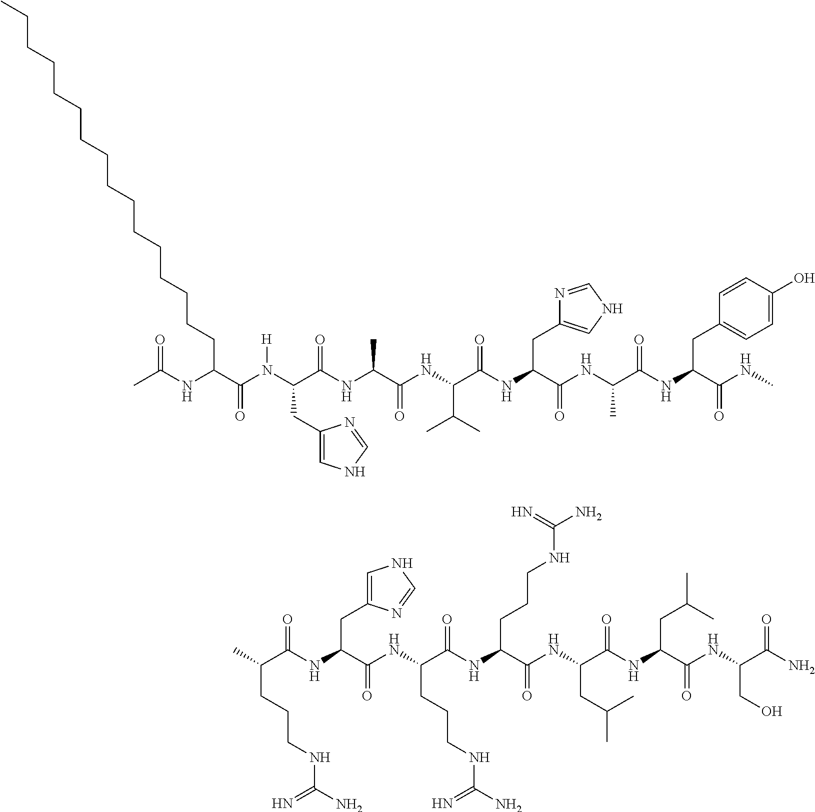 |
| 55 | 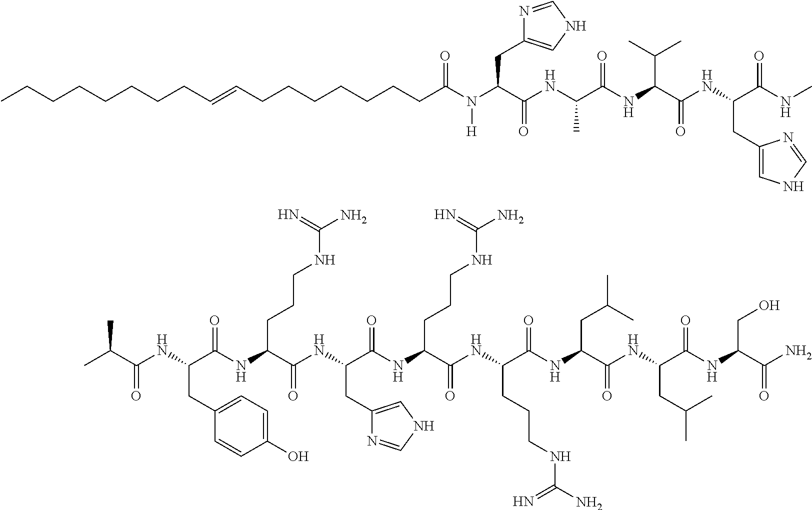 |
| 56 | 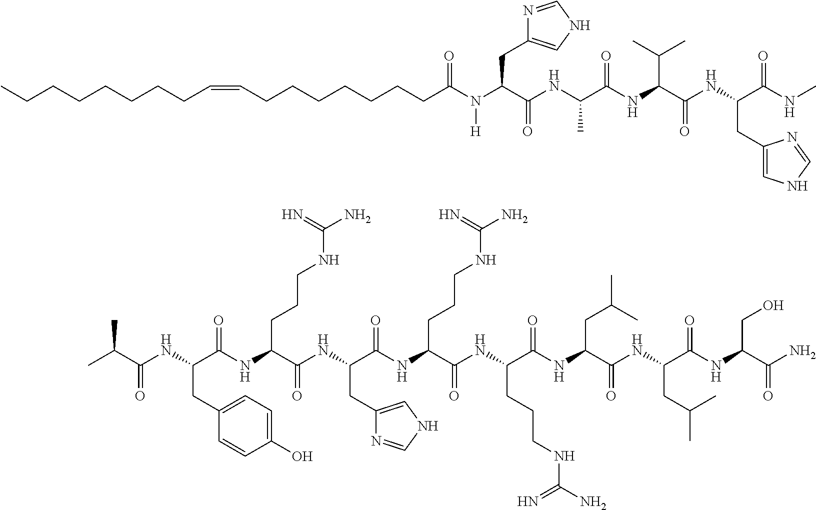 |

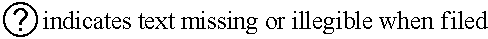

TABLE 16
CXCR5 i3 loop compounds

| Comp. | |
|---|---|
| # | Structure |
| 57 | 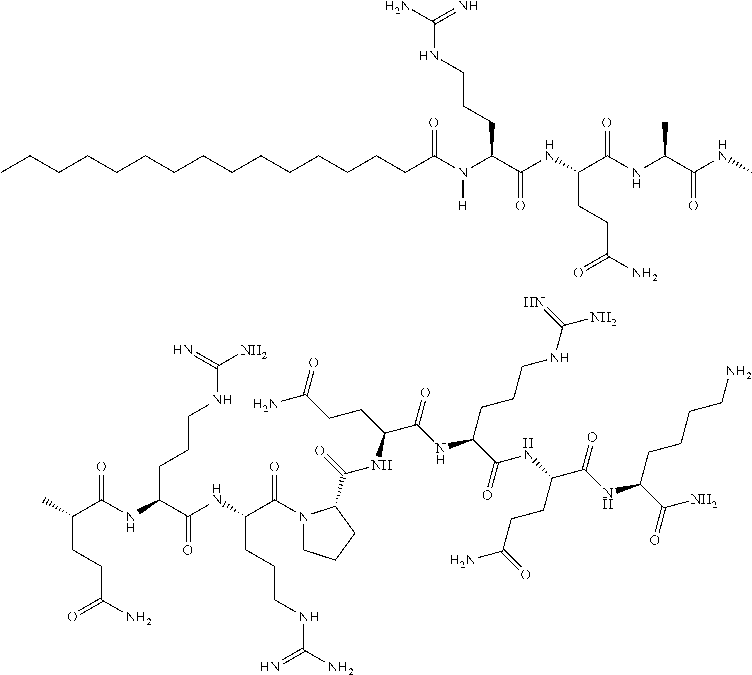 |
| 58 | 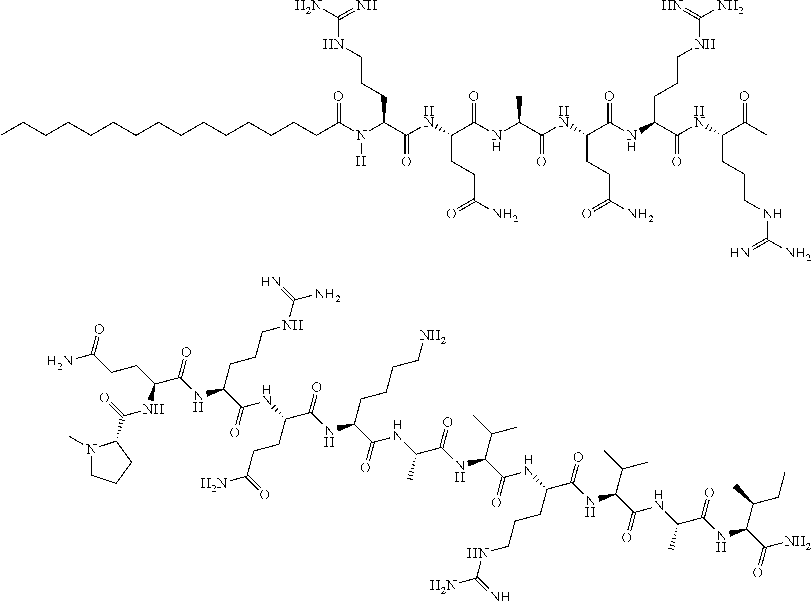 |
| 59 | 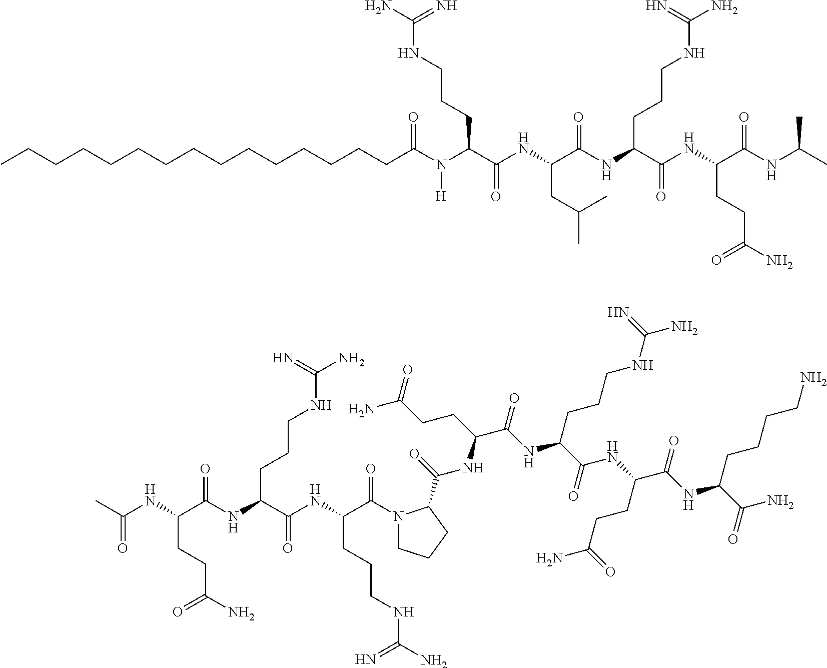 |
| 60 | 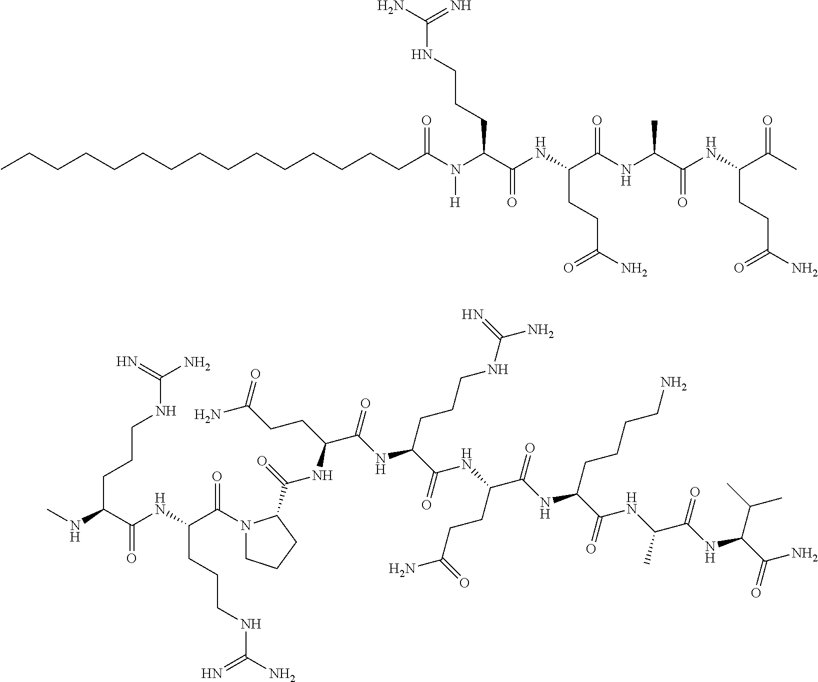 |
| 61 | 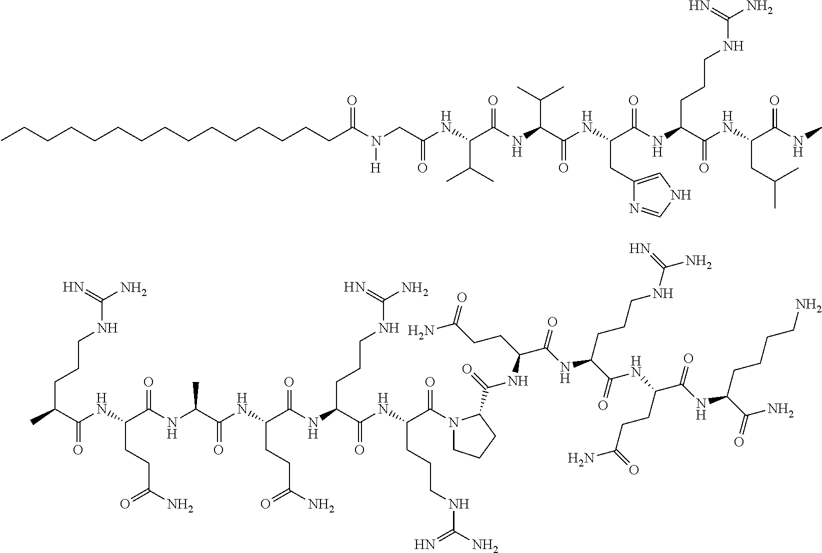 |
| 62 | 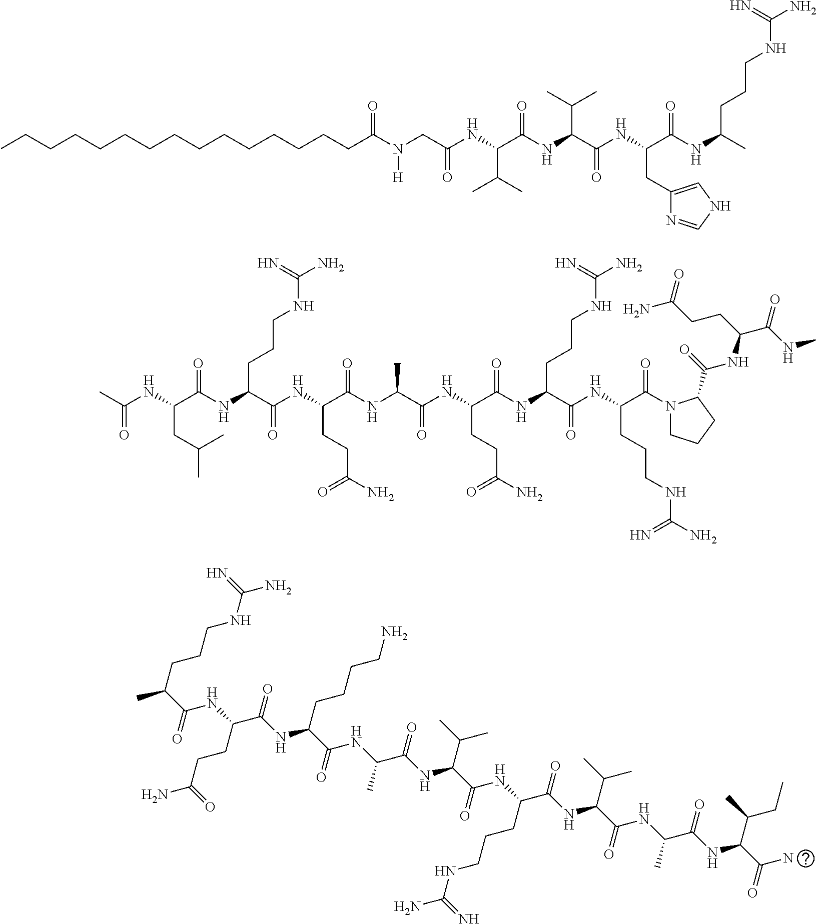 |
| 63 | 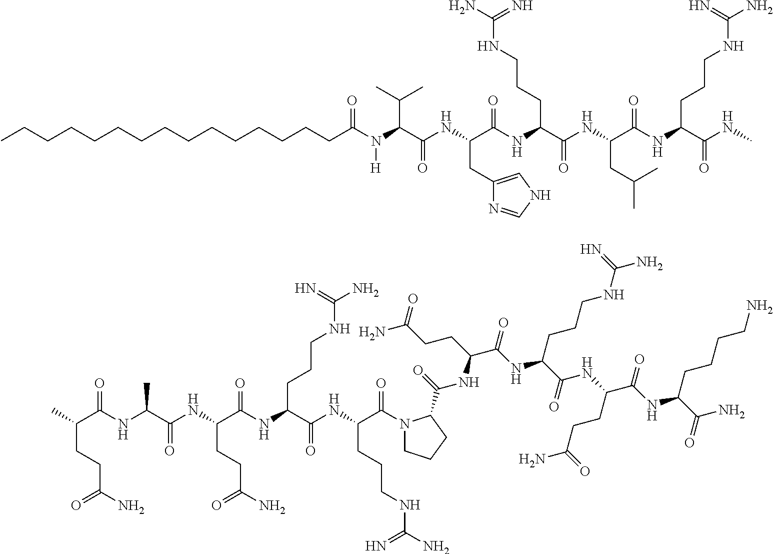 |
| 64 | 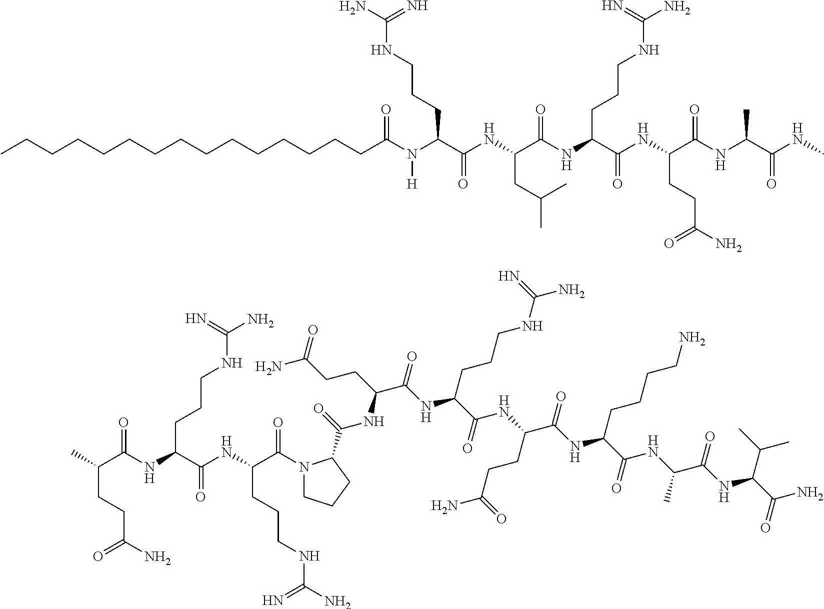 |
| 65 | 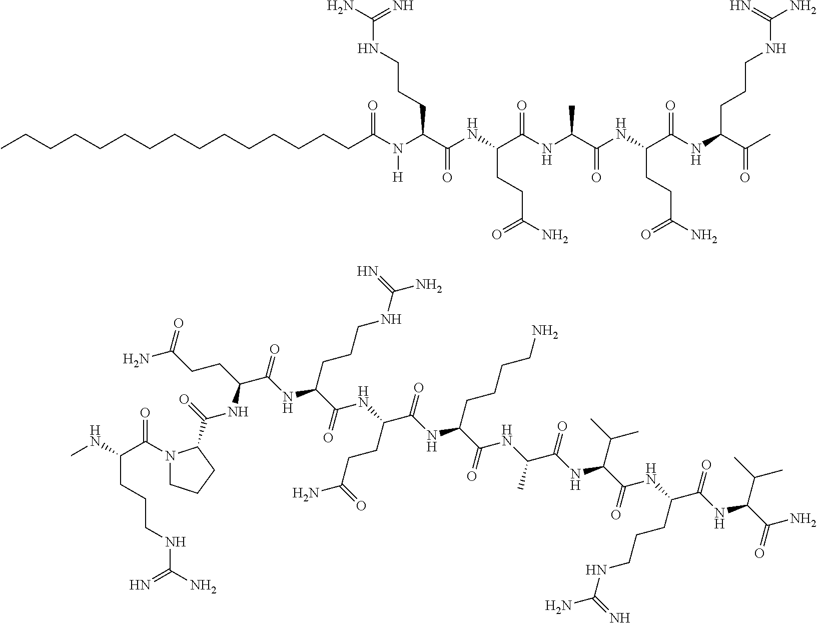 |
| 66 | 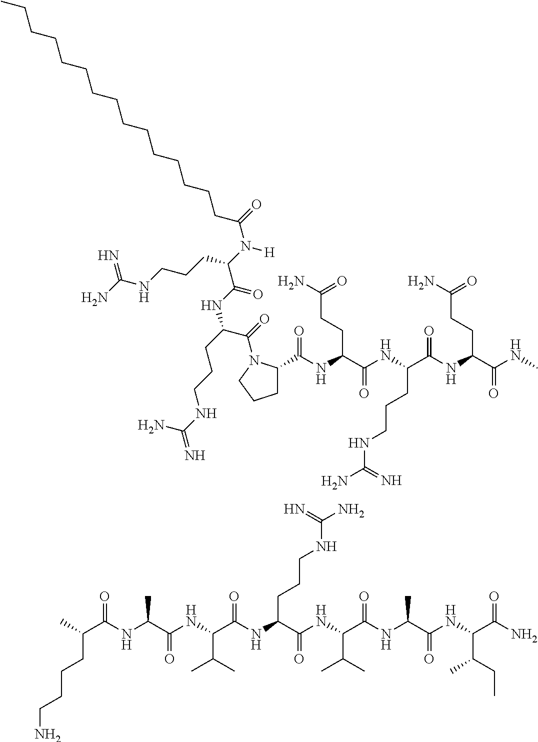 |
| 67 | 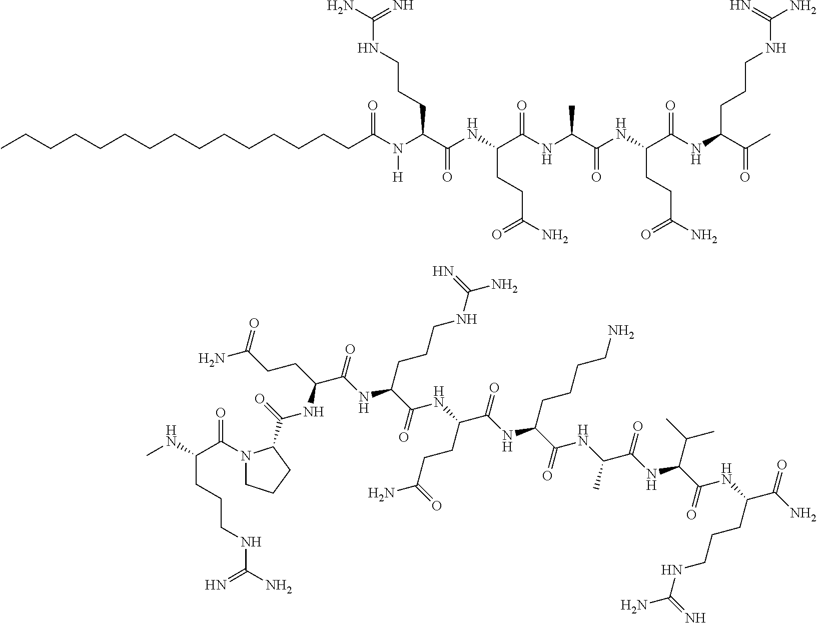 |
| 68 | 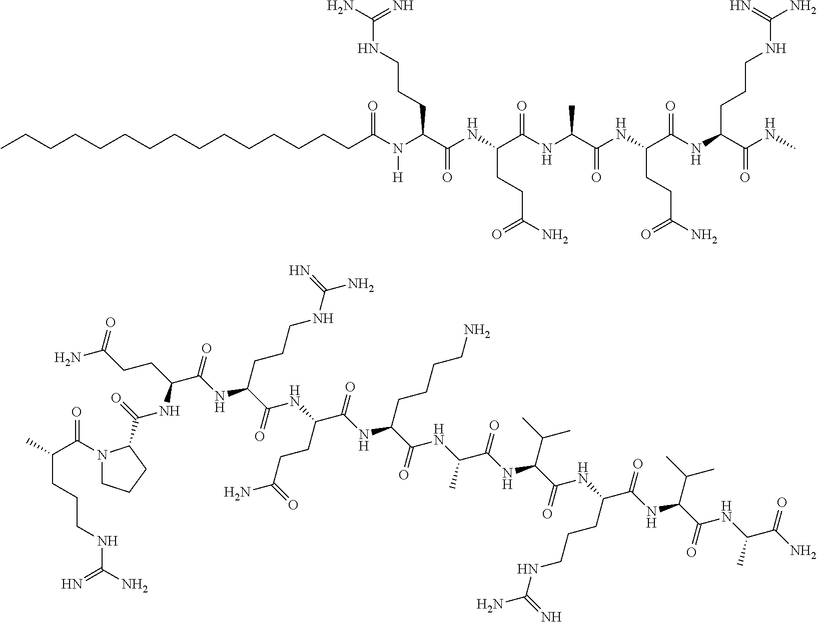 |
| 69 | 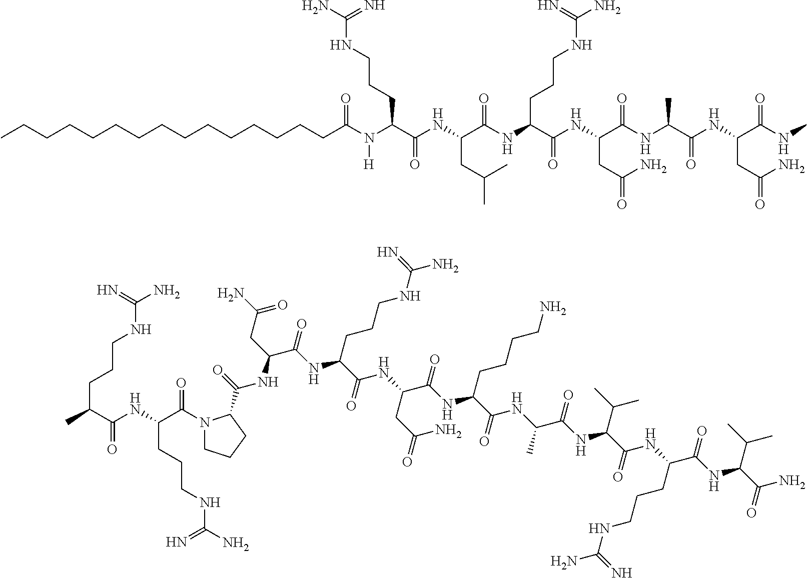 |
| 70 | 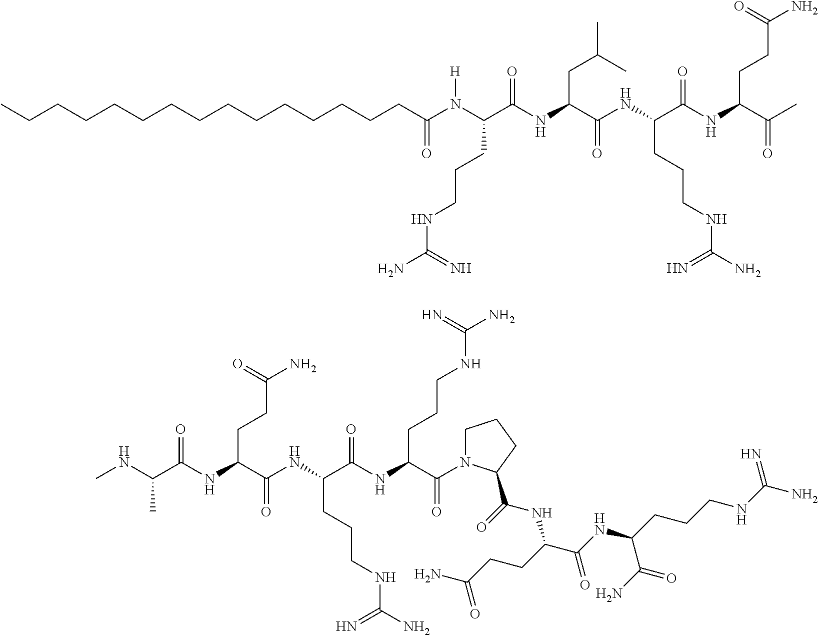 |
| 71 | 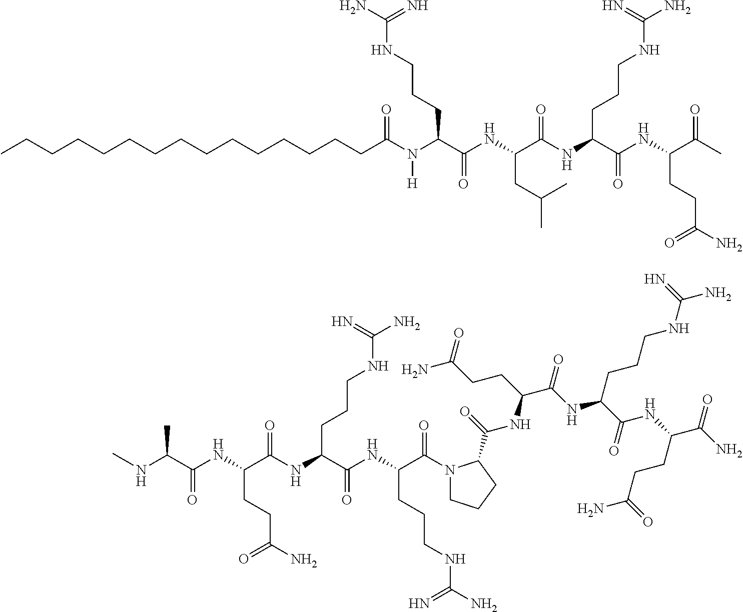 |
| 72 | 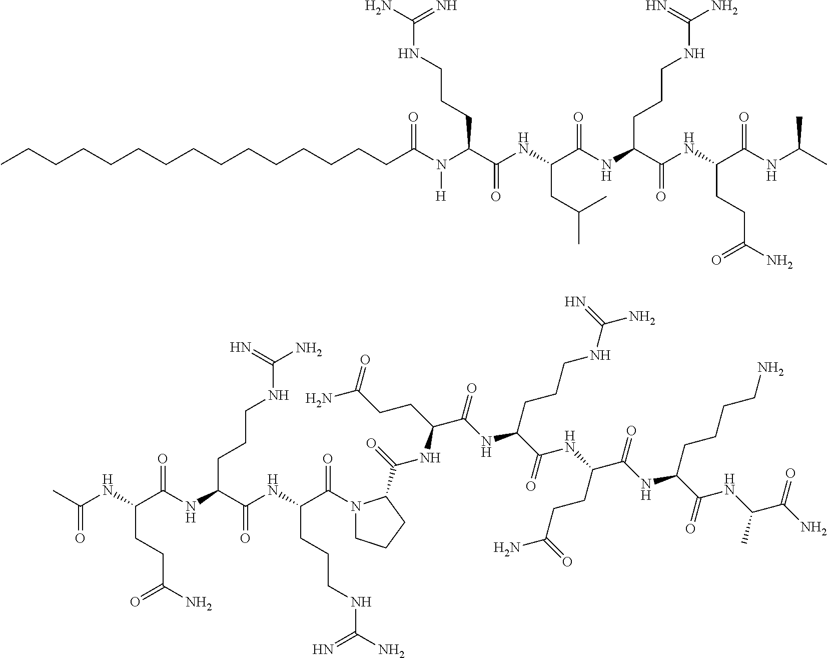 |
| 73 | 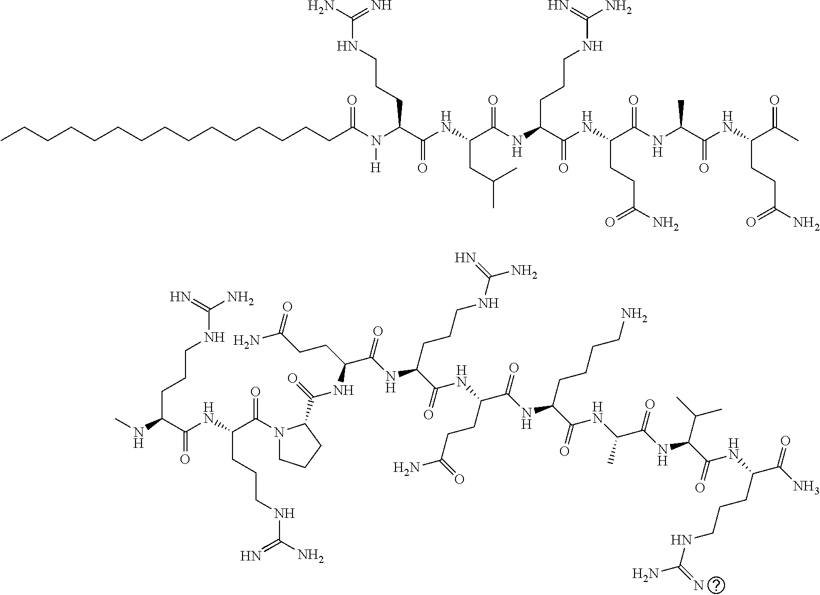 |
| 74 | 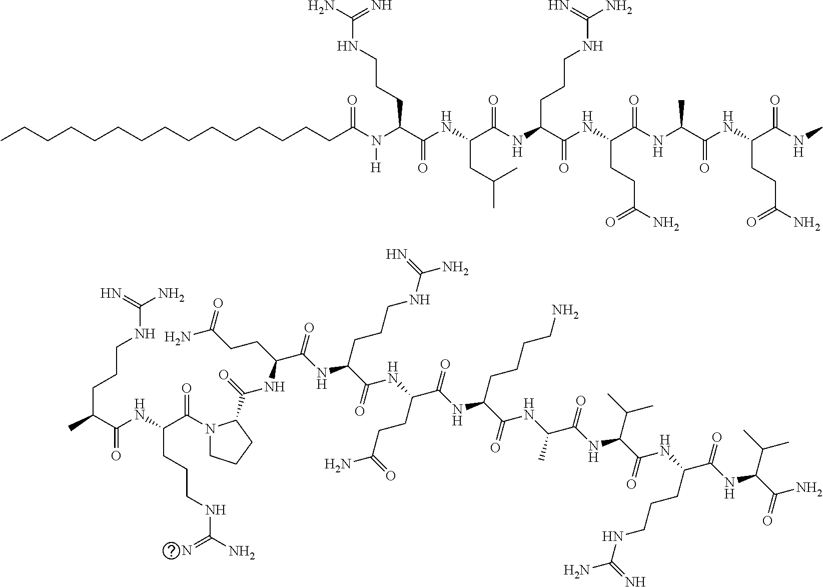 |
| 75 | 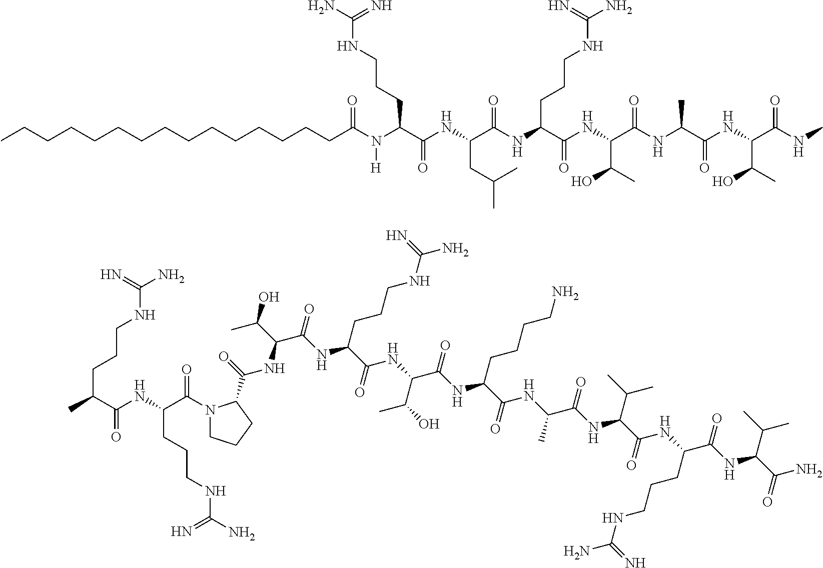 |
| 76 | 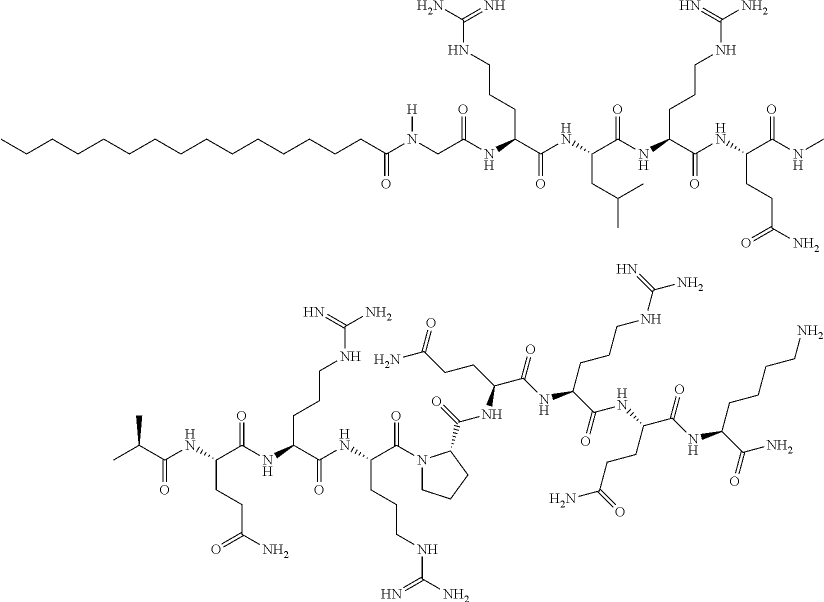 |
| 77 | 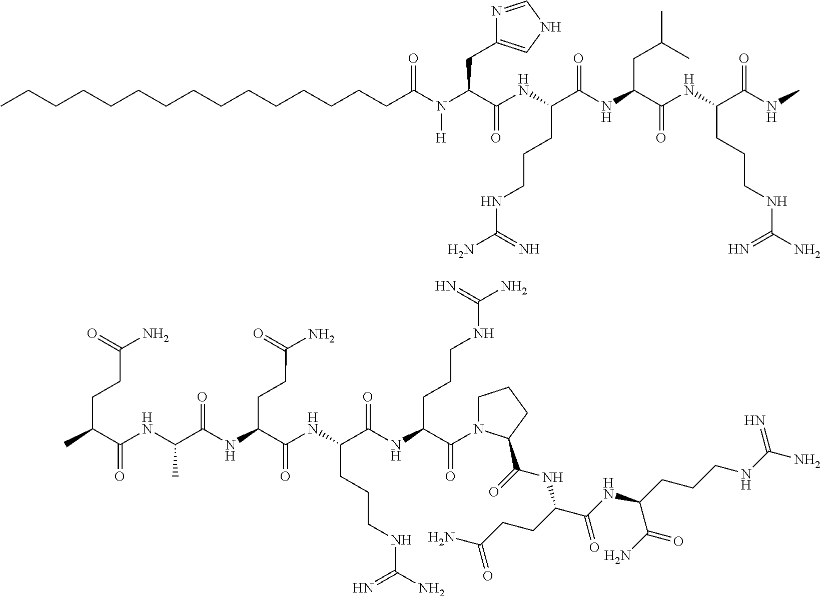 |
| 78 | 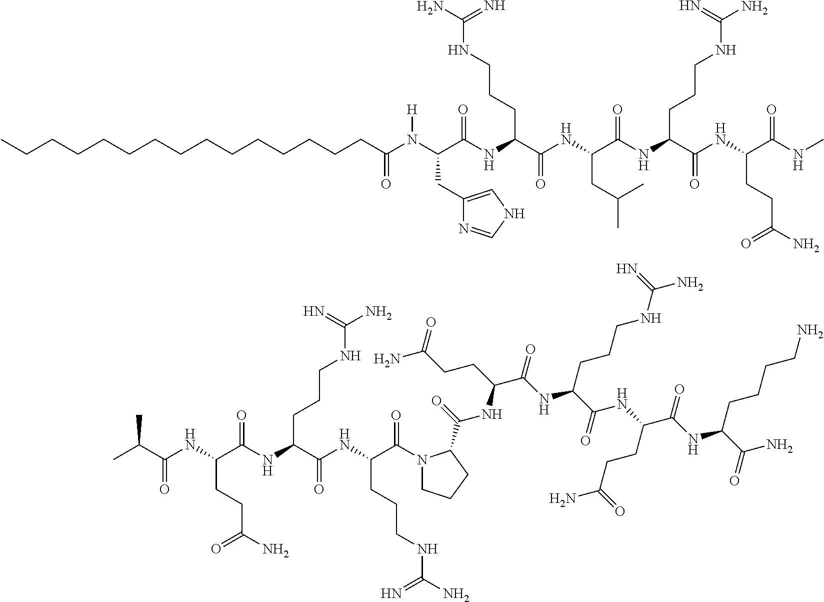 |
| 79 | 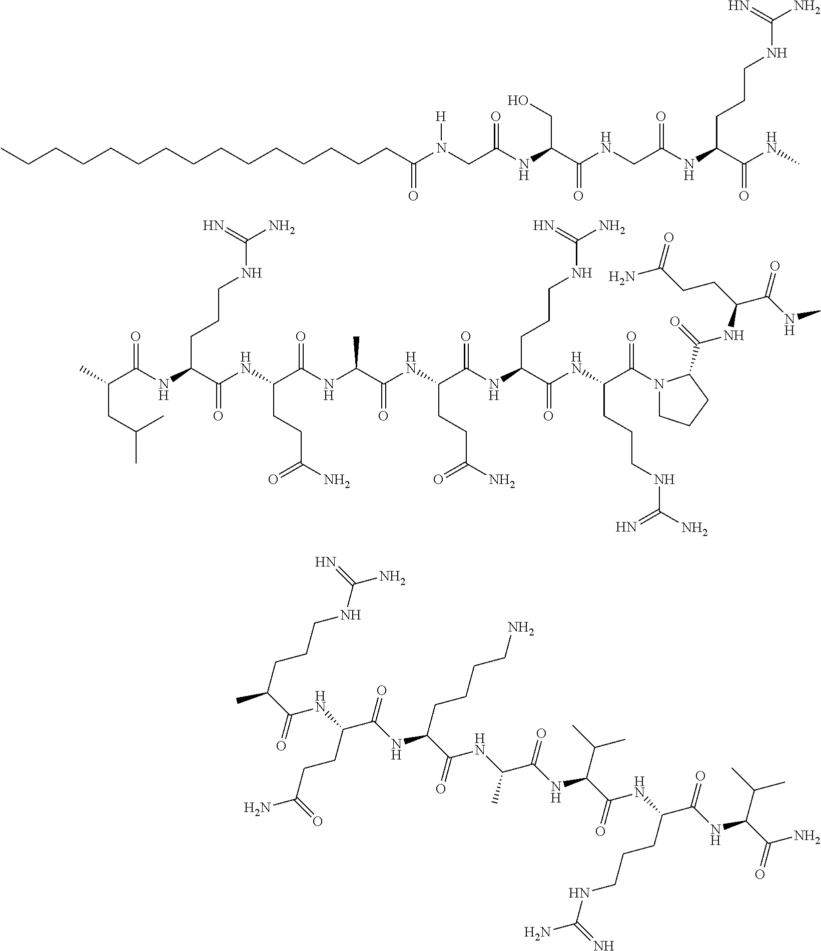 |

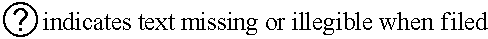

TABLE 17
CXCR5 i4 loop compounds

| Comp. | |
|---|---|
| # | Structure |
| 80 | 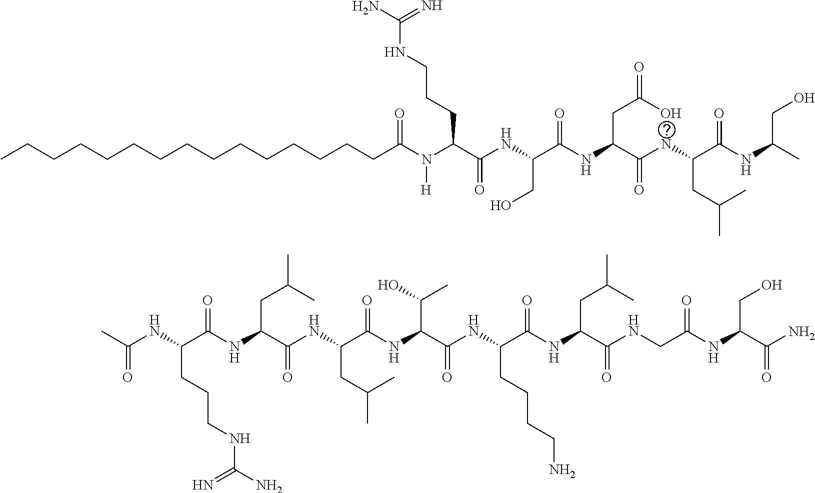 |
| 81 | 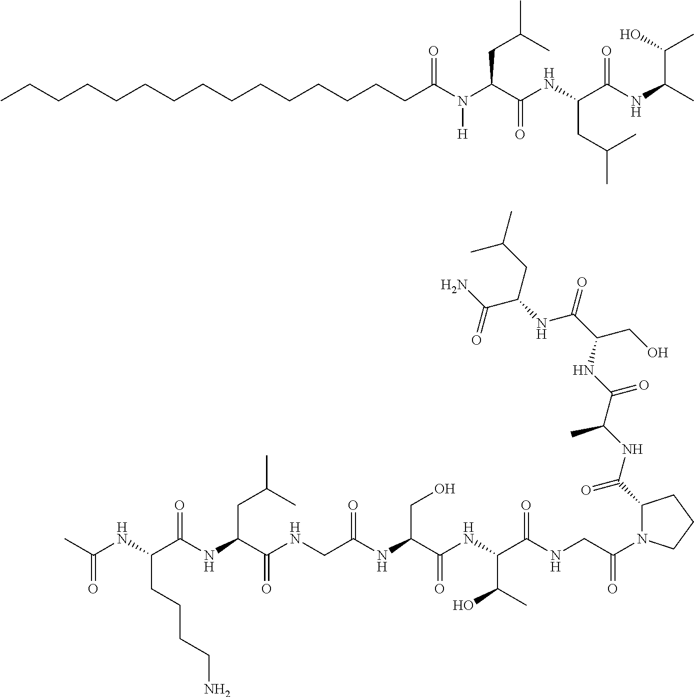 |
| 82 | 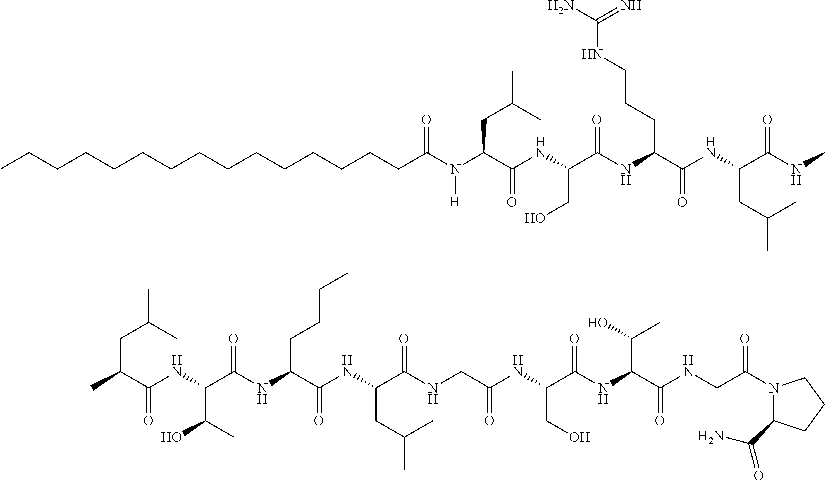 |
| 83 | 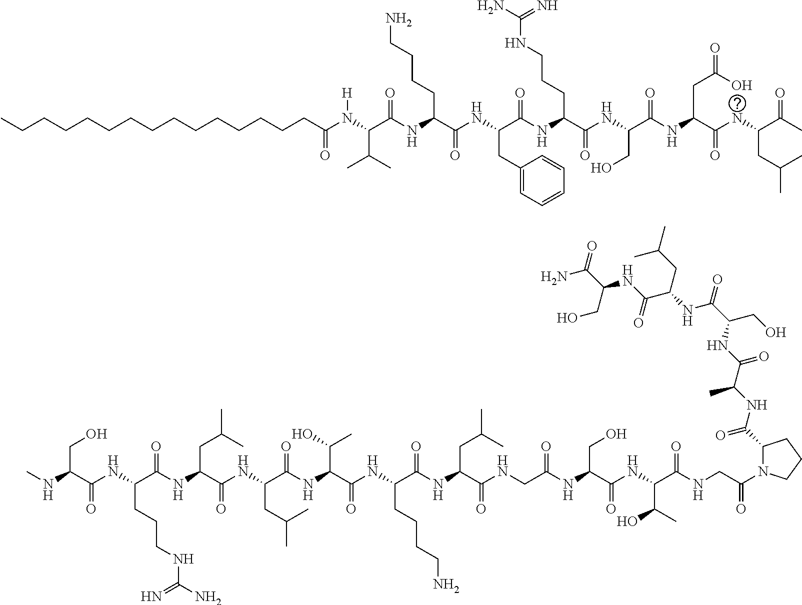 |

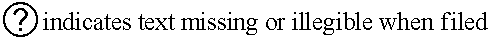

“Cycloalkyl” used alone or as part of a larger moiety such as “cycloalkylalkyl” refers to a monocyclic or polycyclic, non-aromatic ring system of 3 to 20 carbon atoms, 3 to 12 carbon atoms, or 3 to 9 carbon atoms, which may be saturated or unsaturated. Examples of cycloalkyl groups include cyclopropyl, cyclobutyl, cyclopentyl, cyclohexyl, cyclohexenyl, cyclohexa-1,3-dienyl, cyclooctyl, cycloheptanyl, norbornyl, adamantyl, and the like.

“Heterocycloalkyl” refers to a saturated or unsaturated, non-aromatic, monocyclic or polycyclic ring system of 3 to 20 atoms, 3 to 12 atoms, or 3 to 8 atoms, containing one to four ring heteroatoms chosen from O, N and S. Examples of heterocyclyl groups include pyrrolidine, piperidine, tetrahydrofuran, tetrahydropyran, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine-1,1-dioxide, tetrahydro-2H-1,2-thiazine-1,1-dioxide, isothiazolidine-1,1-dioxide, pyrrolidin-2-one, piperidin-2-one, piperazin-2-one, and morpholin-2-one, and the like.

“Halogen” and “halo” refer to fluoro, chloro, bromo or iodo.

“Haloalkyl” refers to an alkyl group substituted with one or more halogen atoms. By analogy, “haloalkenyl”, “haloalkynyl”, etc., refers to the group (for example alkenyl or alkynyl) substituted by one or more halogen atomes.

“Cyano” refers to the group —CN.

“Oxo” refers to a divalent ═O group.

“Thioxo” refers to a divalent ═S group.

“Ph” refers to a phenyl group.

“Carbonyl” refers to a divalent —C(O)— group.

“Alkyl” used alone or as part of a larger moiety such as “hydroxyalkyl”, “alkoxyalkyl”, “alkylamine” refers to a straight or branched, saturated aliphatic group having the specified number of carbons, typically having 1 to 12 carbon atoms. More particularly, the aliphatic group may have 1 to 10, 1 to 8, 1 to 6, or 1 to 4 carbon atoms. This term is exemplified by groups such as methyl, ethyl, n-propyl, isopropyl, n-butyl, isobutyl, tert-butyl, n-hexyl, and the like.

“Alkenyl” refers to a straight or branched aliphatic group with at least one double bond. Typically, alkenyl groups have from 2 to 12 carbon atoms, from 2 to 8, from 2 to 6, or from 2 to 4 carbon atoms. Examples of alkenyl groups include ethenyl (—CH═CH₂), n-2-propenyl (allyl, —CH₂CH═CH₂), pentenyl, hexenyl, and the like.

“Alkynyl” refers to a straight or branched aliphatic group having at least 1 site of alkynyl unsaturation. Typically, alkynyl groups contain 2 to 12, 2 to 8, 2 to 6 or 2 to 4 carbon atoms. Examples of alkynyl groups include ethynyl (—C≡CH), propargyl (—CH₂C≡CH), pentynyl, hexynyl, and the like.

“Alkylene” refers to a bivalent saturated straight-chained hydrocarbon, e.g., C₁-C₆ alkylene includes —(CH₂)₆—, —CH₂—CH—(CH₂)₃CH₃, and the like. “Bivalent means that the alkylene group is attached to the remainder of the molecule through two different carbon atoms.

“Alkenylene” refers to an alkylene group with in which one carbon-carbon single bond is replaced with a double bond.

“Alkynylene” refers to an alkylene group with in which one carbon-carbon single bond is replaced with a triple bond.

“Aryl” used alone or as part of a larger moiety as in “aralkyl” refers to an aromatic carbocyclic group of from 6 to 14 carbon atoms having a single ring or multiple condensed rings. The term “aryl” also includes aromatic carbocycle(s) fused to cycloalkyl or heterocycloalkyl groups. Examples of aryl groups include phenyl, benzo[d][1,3]dioxole, naphthyl, phenantrenyl, and the like.

“Aryloxy” refers to an —OAr group, wherein O is an oxygen atom and Ar is an aryl group as defined above.

“Aralkyl” refers to an alkyl having at least one alkyl hydrogen atom replaced with an aryl moiety, such as benzyl, —(CH₂)₂phenyl, —(CH₂)₃phenyl, —CH(phenyl)₂, and the like.

“Alkyl cycloalkyl” refers to an alkyl having at least one alkyl hydrogen atom replaced with a cycloalkyl moiety, such as —CH₂-cyclohexyl, —CH₂-cyclohexenyl, and the like.

“Heteroaryl” used alone or a part of a larger moiety as in “heteroaralkyl” refers to a 5 to 14 membered monocyclic, bicyclic or tricyclic heteroaromatic ring system, containing one to four ring heteroatoms independently selected from nitrogen, oxygen and sulfur. The term “heteroaryl” also includes heteroaromatic ring(s) fused to cycloalkyl or heterocycloalkyl groups. Particular examples of heteroaryl groups include optionally substituted pyridyl, pyrrolyl, pyrimidinyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl,1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 1,2,3,4-tetrahydroquinolyl, 1,2,3,4-tetrahydroisoquinolyl, purinyl, pteridinyl, carbazolyl, xanthenyl, benzoquinolyl, and the like.

“Heteroaryloxy” refers to an —OHet group, wherein O is an oxygen atom and Het is a heteroaryl group as defined above.

“Heteroaralkyl” refers to an alkyl having at least one alkyl hydrogen atom replaced with a heteroaryl moiety, such as —CH₂-pyridinyl, —CH₂-pyrimidinyl, and the like.

“Alkoxy” refers to the group —O—R where R is “alkyl”, “cycloalkyl”, “alkenyl”, or “alkynyl”. Examples of alkoxy groups include for example, methoxy, ethoxy, ethenoxy, and the like.

“Alkyl heterocycloalkyl” refers to an alkyl having at least one alkyl hydrogen atom replaced with a heterocycloalkyl moiety, such as —CH₂-morpholino, —CH₂-piperidyl and the like.

“Alkoxycarbonyl” refers to the group, —C(O)OR where R is “alkyl”, “alkenyl”, “alkynyl”, “cycloalkyl”, “heterocycloalkyl”, “aryl”, or “heteroaryl”.

“Hydroxyalkyl” and “alkoxyalkyl” are alky groups substituted with hydroxyl and alkoxy, respectively.

“Amino” means —NH₂; “alkylamine” and “dialkylamine” mean —NHR and —NR₂, respectively, wherein R is an alkyl group. “Cycloalkylamine” and “dicycloalkylamine” mean —NHR and —NR₂, respectively, wherein R is a cycloalkyl group. “Cycloalkylalkylamine” means —NHR wherein R is a cycloalkylalkyl group. “[Cycloalkylalkyl][alkyl]amine” means —N(R)₂ wherein one R is cycloalkylalkyl and the other R is alkyl.

Haloalkyl and halocycloalkyl include mono, poly, and perhaloalkyl groups where the halogens are independently selected from fluorine, chlorine, bromine and iodine.

Suitable substituents for “alkyl”, “alkenyl”, “alkynyl”, “cycloalkyl”, “heterocycloalkyl”, “aryl”, or “heteroaryl”, etc., are those which will form a stable compound of the invention. Examples of suitable substituents are those selected from the group consisting of halogen, —CN, —OH, —NH₂, (C₁-C₄)alkyl, (C₁-C₄)haloalkyl, aryl, heteroaryl, (C₃-C₇)cycloalkyl, (5-7 membered) heterocycloalkyl, —NH(C₁-C₆)alkyl, —N((C₁-C₆)alkyl)₂, (C₁-C₆)alkoxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)NH(C₁-C₆)alkyl)₂, —N(C₁-C₆)alkylC(S)N((C₁-C₆)alkyl)₂, —CONH(C₁-C₆)alkyl, —OCONH(C₁-C₆)alkyl —CON((C₁-C₆)alkyl)₂, —C(S)(C₁-C₆)alkyl, —S(O)ₚ(C₁-C₆)alkyl, —S(O)ₚNH₂, —S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H. More particularly, the substituents are selected from halogen, —CN, —OH, —NH₂, (C₁-C₄)alkyl, (C₁-C₄)haloalkyl, (C₁-C₄)alkoxy, phenyl, and (C₃-C₇)cycloalkyl. Within the framework of this invention, said “substitution” is also meant to encompass situations where a hydrogen atom is replaced with a deuterium atom. p is an integer with a value of 1 or 2.

Pharmaceutically acceptable salts of the compounds disclosed herein are included in the present invention. For example, an acid salt of a compound containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

Salts of the compounds containing an acidic functional group can be prepared by reacting with a suitable base. Such a pharmaceutically acceptable salt can be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

### Pharmaceutical Compositions

The invention also provides pharmaceutical compositions comprising an effective amount of a compound Formula, I (e.g., including any of the formulae herein), or a pharmaceutically acceptable salt of said compound; and a pharmaceutically acceptable carrier. The carrier(s) are “pharmaceutically acceptable” in that they are not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. Nos. 7,014,866; and U.S. Patent Publications US 2006/0094744 and US 2006/0079502.

The pharmaceutical compositions of the invention include those suitable for oral, rectal, nasal, topical (including buccal and sublingual), pulmonary, vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable'vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the patient therapeutics may be local, so as to be administered at the site of interest. Various techniques can be used for providing the patient compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. In one embodiment, the second therapeutic agent is one or more additional compounds of the invention.

In another embodiment, the second therapeutic agent may be selected from any compound or therapeutic agent known to have or that demonstrates advantageous properties when administered with a compound having the same mechanism of action as the CXCR5 receptor compound of Formula I.

In a particular embodiment, the second therapeutic is an agent useful in the treatment or prevention of a disease or condition selected from autoimmune diseases such as lupus, HIV and rheumatoid arthritis, Primary Sjögren's Syndrome, chronic lymphocytic leukemia, Burkitt Lymphoma, colon and breast cancer tumor metastasis, Multiple Sclerosis and compromised immune function.

In another embodiment, the second therapeutic is an agent useful in the treatment or prevention of a disease or condition selected from from autoimmune diseases such as lupus.

In one embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy. Preferably, the compound is present in the composition in an amount of from 0.1 to 50wt. %, more preferably from 1 to 30 wt. %, most preferably from 5 to 20wt. %.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily treatment dose or one of multiple daily treatment doses (e.g., about 1 to 4 or more times per day). When multiple daily treatment doses are used, the unit dosage form can be the same or different for each dose.

### Methods of Treatment

As used herein the term “subject” and “patient” typically means a human, but can also be an animal in need of treatment, e.g., companion animals (dogs, cats, and the like), farm animals (cows, pigs, horses, sheep, goats, and the like) and laboratory animals (rats, mice, guinea pigs, and the like).

The terms “treat” and “treating” are used interchangeably and include both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The invention also includes methods of treating diseases, disorders or pathological conditions which benefit from modulation of the CXCR5 receptor comprising administering an effective amount of a CXCR5 receptor compound of the invention to a subject in need thereof. Diseases and conditions which can benefit from modulation (inhibition or activation) of the CXCR5 receptor include, but are not limited to, autoimmune diseases such as lupus, HIV and rheumatoid arthritis, Primary Sjögren's Syndrome, chronic lymphocytic leukemia, Burkitt Lymphoma, colon and breast cancer tumor metastasis, Multiple Sclerosis and compromised immune function.

More specifically, CXCR5 mediated signal transduction is an important nexus of the trafficking and homing of B1 cells. In chronic lymphocytic leukemia (CLL) B cells express high levels of functional CXCR5. In addition, CLL patients express significantly higher serum levels of the canonical endogenous chemokine Ligand for CXCR5-CXCL13 (also called BLC, BCA-1) than control groups. Overall, data suggest that CXCR5 signaling plays a role in CLL cell position and cognate interactions between CLL and CXCL13-secreting CD68+ accessory cells in lymphoid tissues. Antagonistic or negative regulatory GPCR compounds of the invention derived from CXCR5 could be utilized to modulate this system, providing a new therapeutic avenue to pursue in the treatment of CLL and other lymphoma and leukemias.

In one embodiment, an effective amount of a compound of this invention can range from about 0.005 mg to about 5000 mg per treatment. In more specific embodiments, the range is from about 0.05 mg to about 1000 mg, or from about 0.5 mg to about 500 mg, or from about 5 mg to about 50 mg. Treatment can be administered one or more times per day (for example, once per day, twice per day, three times per day, four times per day, five times per day, etc.). When multiple treatments are used, the amount can be the same or different. It is understood that a treatment can be administered every day, every other day, every 2 days, every 3 days, every 4 days, every 5 days, etc. For example, with every other day administration, a treatment dose can be initiated on Monday with a first subsequent treatment administered on Wednesday, a second subsequent treatment administered on Friday, etc. Treatment is typically administered from one to two times daily. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

Alternatively, the effective amount of a compound of the invention is from about 0.01 mg/kg/day to about 1000 mg/kg/day, from about 0.1 mg/kg/day to about 100 mg/kg/day, from about 0.5 mg/kg/day about 50 mg/kg/day, or from about 1 mg/kg/day to 10 mg/kg/day.

In another embodiment, any of the above methods of treatment comprises the further step of co-administering to said patient one or more second therapeutic agents. The choice of second therapeutic agent may be made from any second therapeutic agent known to be useful for co-administration with a compound that modulates the CXCR5 receptor. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

### Kits

The present invention also provides kits for use to treat the target disease, disorder or condition. These kits comprise (a) a pharmaceutical composition comprising a compound of Formula I, or a salt thereof, wherein said pharmaceutical composition is in a container; and (b) instructions describing a method of using the pharmaceutical composition to treat the target disease, disorder or condition.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### Genreal Methods for Preparing CXCR5 Receptor Compounds

### Synthesis of Peptides

The peptide component (P) of the compounds of the invention can be synthesized by incorporating orthogonally protected amino acids in a step-wise fashion. Any suitable synthetic methods can be used. Traditional Fmoc or Boc chemistry can be easily adapted to provide the desired peptide component (P) of the compounds of the invention. Fmoc is generally preferred, because the cleavage of the Fmoc protecting group is milder than the acid deprotection required for Boc cleavage, which requires repetitive acidic deprotections that lead to alteration of sensitive residues, and increase acid catalyzed side reactions.(G. B. FIELDS et al. in Int. J. Pept. Protein, 1990, 35, 161).

The peptides can be assembled linearly via Solid Phase Peptide Synthesis (SPPS), can be assembled in solution using modular condensations of protected or unprotected peptide components or a combination of both.

### Solid Phase Peptide Synthesis

For SPPS, an appropriate resin is chosen that will afford the desired moiety on the C-terminus upon cleavage. For example upon cleavage of the linear peptide, a Rink amide resin will provide a primary amide on the C-terminus, whereas a Rink acid resin will provide an acid. Rink acid resins are more labile than Rink amide resins and the protected peptide could also be cleaved and subsequently the free acid activated to react with amines or other nucleophiles. Alternatively, other resins could provide attachment of other moieties prior to acylation, leading to cleavage of an alkylated secondary amide, ester or other desired C-terminal modification. A review of commonly used resins and the functional moiety that results after cleavage can be found in manufacturer literature such as NovaBiochem or Advanced Chemtech catalogues.

Typically a resin is chosen such that after cleavage the C-terminus is an amide bond. Rink amide resin is a resin that results in a C-terminal amide during cleavage. The orthogonally protected Fmoc amino acids are added stepwise using methods well known in literature (Bodansky M. Principles of Peptide synthesis (1993) 318p; Peptide Chemistry, a Practical Textbook (1993); Spinger-Verlag). These procedures could be done manually or by using automated peptide synthesizers.

The process involves activating the acid moiety of a protected amino acid, using activating agents such as HBTU, HATU, PyBop or simple carbodiimides. Often an additive is used to decrease racemization during coupling such as HOBt or HOAt (M. SCHNÖLZER et al., Int. J. Pept. Protein Res., 1992, 40, 180). Manually, the coupling efficiency can be determined photometrically using a ninhydrin assay. If the coupling efficiency is below 98%, a second coupling may be desired. After the second coupling a capping step may be employed to prevent long deletion sequences to form, simplifying the purification of the desired final compound. With automation, second couplings are not commonly required, unless a residue is known to be problematic such as Arginine.

Deprotection of the Fmoc is most commonly accomplished using piperidine (20%) in dimethylformamide (DMF). Alternatively other secondary amines may also be used such as morpholine, diethylamine or piperazine. This reaction is facile and normally is accomplished within 20 minutes using piperidine. After deprotection the resin is washed several times with DMF and DCM prior to coupling with the next residue. This process is repeated, assembling the peptide linearly until the sequence is complete. The final Fmoc is removed, which allows for coupling with the tether moiety.

In a preferred synthesis, the peptide is formed by SPPS accomplished manually or in an automated fashion using a commercially available synthesizer such as the CEM Microwave peptide synthesizer, Rainin Symphony synthesizer, or ABI 433 flow-through synthesizer. Commercially available Rink Amide resin is used for synthesizing the C-terminal amide peptides (Rink, H. Tetrahedron Lett, 28, 4645, 1967). Peptide synthesis reagents (coupling, deprotection agents) are commercially available and include HOBT, HBTU (Novabiochem) as well as DMF, DCM, Piperidine, NMP, and DIEA (Sigma-Aldrich). Suitably protected amino acids for use in solid phase peptide synthesis are commercially available from many sources, including Sigma-Aldrich and CEM Corporation.

For example, a convenient preparation of peptides on a 0.1 mmol or 0.25 mmol scale uses Rink amide solid-phase resin with a substitution of about 0.6 mmol/g. Linear attachment of the amino acids is accomplished on a ABI continuous flow automated synthesizer using 5 eq of orthogonally protected amino acid (AA), and using HBTU/HOBt coupling protocol, (5 eq. of each reagent). In another preferred synthesis, peptides can be synthesized using a microwave instrument using 10 eq of reagents. Deprotection of Fmoc can be accomplished with 20% piperidine in DMF followed by washing with DMF and DCM.

In both cases (i.e., Rink acid and Rink amide resins), final Fmoc deprotection of the N-terminus would leave a free amine after cleavage from the resin unless it is modified prior to cleavage. In the compounds of the invention, tether moieties are attached through amide bonds.

### Solution Phase Synthesis of Peptides

For solution phase synthesis the desired peptide is generally broken down into peptide fragments in units of 2-4 amino acids. The selected unit is dependent on the sequence, the stability of the fragment to racemization, and the ease of assembly. As each amino acid is added, only 1-1.5eq of the residue is required, versus the 5-10 equivalents of reagent required for SSPS. Preactivated amino acids such as OSu active ester and acid fluorides also can be used, requiring only a base for completion of the reaction.

Coupling times require 1.5-2 hours for each step. Two fragments are condensed in solution, giving a larger fragment that then can be further condensed with additional fragments until the desired sequence is complete. The solution phase protocol uses only 1 eq of each fragment and will use coupling reagents such as carbodiimides (DIC). For racemized prone fragments, PyBop or HBTU/HOBt can be used. Amino acids with Bsmoc/tBu or Fmoc/tBu and Boc/Benzyl protection are equally suitable for use.

When Fmoc is used, the use of 4-(aminomethyl) piperidine or tris(2-aminoethyl)amine as the deblocking agent can avoid undesired side reactions. The resulting Fmoc adduct can be extracted with a phosphate aqueous buffer of pH 5.5 (Organic Process Research & Development 2003, 7, 2837). If Bsmoc is used, no buffer is required, only aqueous extractions are needed. Deprotections using these reagents occur in 30-60 minutes. Deblocking of the Fmoc group on the N-terminal residue provides a free terminal amine that is used for attachment of the tether moiety. In the compounds of the invention, tether moieties are attached through amide bonds to the N-terminal amine.

One advantage of solution phase synthesis is the ability to monitor the compound after every coupling step by mass spectrometry to see that the product is forming. In addition, a simple TLC system could be used to determine completion of reaction.

### Attachment of Tethers

Tethers are attached to the terminal nitrogen of the N-terminal amino acid of the peptide chain using amide bond coupling:

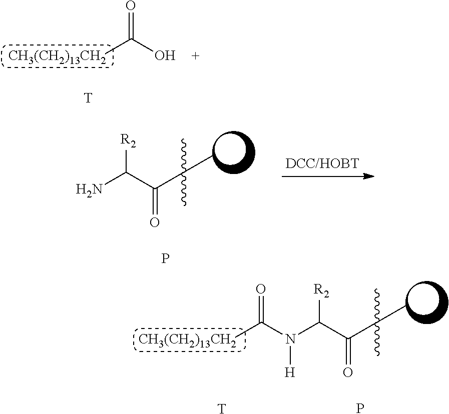

The tether can be attached using solid phase procedures or in solution using an amide bond coupling. After the N-terminus is suitably coupled, the final compound is cleaved from the resin using an acidic cocktail (Peptide Synthesis and Applications, John Howl, Humana Press, 262p, 2005). Typically these cocktails use concentrated trifluoroacetic acid (80-95%) and various scavengers to trap carbocations and prevent side chain reactions. Typical scavengers include isopropylsilanes, thiols, phenols and water. The cocktail mixture is determined by the residues of the peptide. Special care needs to be taken with sensitive residues, such as methionine, aspartic acid, and cysteine. Typical deprotection occurs over 2-5 hours in the cocktail. A preferred deprotection cocktail include the use of triisopropylsilane (TIS), Phenol, thioanisole, dodecanethiol (DDT) and water. Methane sulfonic acid (MSA) may also be used in the cocktail (4.8%). A more preferred cocktail consists of (TFA:MSA:TIS:DDT:Water 82: 4.5:4.5:4.5:4.5; 10 mL/0.1 mmol resin).

After deprotection, the resin is removed via filtration, and the final compound is isolated via precipitation from an organic solvent such as diethyl ether, m-tert-butyl ether, or ethyl acetate and the resulting solid collected via filtration or lyophilized to a powder. Purification of the peptide using reverse phase HPLC may be required to achieve sufficient purity. Generally, a gradient of aqueous solvent with an organic solvent will provide sufficient separation from impurities and deletion sequences. Typically 0.1% TFA is used as the aqueous and organic modifier, however, other modifiers such as ammonium acetate can also be used. After purification, the compound is collected, analyzed and fractions of sufficient purity are combined and lyophilized, providing the compound as a solid.

### Amino Acid Reagents

The following commercially available orthogonally protected amino acids used can be used in the synthesis of compounds of the invention: Fmoc-Tyr(tBu)-OH, Fmoc-Ala-OH*H₂O, Fmoc-Arg(Pbf)-OH, Fmoc, Asn(Trt)-OH, Fmoc-Asp(tBu), Fmoc-Cys(tBu)-OH, Fmoc-Glu(tBu)-OH, Fmoc-Glx(Pbf)-OH, Fmoc-Gly-OH, Fmoc-His(Trt)-OH, Fmoc-Leu-OH, Fmoc-Ile-OH, Fmoc, Lys(tBu)-OH, Fmoc-Met-OH, Fmoc-Phe-OH, Fmoc-Ser(tBu)-OH, Fmoc-Thr(tBu)-OH, Fmoc-Typ-OH, and Fmoc-Val-OH. Additional amino acids suitable for incorporation into the compounds of the invention (e.g., D amino acids, substituted amino acids and other protecting group variations) are also commercially available or synthesized by methods known in the art.

### Analytical Methods

The compounds of the invention are analyzed for purity by HPLC using the methods listed below. Purification is achieved by preparative HPLC.

Fast LC/MS Method

Column: Phenomenex Luna C-5 20×30 mm

Flow: 1.0 ml/min

Solvent A: 0.1% TFA in Type I water

Solvent B: 0.1% TFA in Acetonitrile

UV 220 nm

Injection: 20 ul

Gradient 5-95% B (7 minutes); 95-5% B (1 minute); 5% B (4 minutes)

Analytical Purity Method

Column: Phenomenex Luna C-5 20×30 mm

Flow: 1.0 ml/min

Solvent A: 0.1% TFA in Type I water

Solvent B: 0.1% TFA in Acetonitrile

UV: 220 nm

Injection: 20 ul

Gradient: 2-95% B (10 minutes); 95-2% B (2 minutes); 2% B (2 minutes)

Preparative LC/MS Method

Column: Phenomenex Luna C-5 250×150 mm

Flow: 5.0 ml/min

Solvent A: 0.1% TFA in Type I water

Solvent B: 0.1% TFA in Acetonitrile

UV: 220 nm

Injection: 900 ul

Gradient: 35% B (5 minutes); 35-85% B (13 minutes); 85-35% B (0.5 minutes); 35% B (1.5 minutes)

### Synthesis of Selected Compounds

| | |
|---|---|
| Compound 14 | Pal-HAVHAYRHRRLLS-amide |

Compound 14 was synthesized as described above on Rink amide resin at 0.1 mmol scale. Amino acids were coupled sequentially as described above. Following deprotection of the Fmoc group on the N-terminal residue serine, the N-terminal amine was capped with palmitic acid (10 eq.), HBTU (10 eq.) and DIEA (10 eq.) as described above. The compound was cleaved from the resin by TFA containing MS, TIS, DDT, and water (82: 4.5:4.5:4.5:4.5; 10 mL), filtered through a Medium frit Buchner full, triturated with ether and the resulting precipitate collected by centrifugation. Crude peptide was taken up in minimum amount of DMSO and purified by RP-HPLC as described previously. Fractions with correct MW were pooled and lyophilized and analyzed for purity using Method A. The yield of representative lots is illustrated in the following table.

| Lot # | Yield (mg) |
|---|---|
| 1 | 9 |
| 2 | 6.2 |
| 3 | 23 |
| 4 | 16.1 |
| 5 | 6.1 |

| | |
|---|---|
| Compound 62 | Pal-GVVHRLRQAQRRPQRQKAVRVAI-amide |

Compound 62 was synthesized as described for Compound 14. The yield representative lots is illustrated in the following table.

| Lot # | Yield (mg) |
|---|---|
| 1 | 7 |
| 2 | 5 |
| 3 | 5.6 |
| 4 | 12.6 |

| | |
|---|---|
| Compound 75 | Pal-RLRTATRRPTRTKAVRV-amide |

Compound 75 was synthesized as described for Compound 14. The yield of representative lots is illustrated in the following table.

| Lot # | Yield (mg) |
|---|---|
| 1 | 9 |

| | |
|---|---|
| Compound 94 | Pal-RQTRSSTETFL-amide |

Compound 94 was synthesized as described for Compound 14. The yield of representative lots is illustrated in the following table.

| Lot # | Yield (mg) |
|---|---|
| 1 | 5.4 |

### Methods of Screening

### Functional Assays

Functional assays suitable for use in detecting and characterizing GPCR signaling include Gene Reporter Assays and Calcium Flux assays, cAMP and kinase activation assays. Several suitable assays are described in detail below.

### Gene Reporter Assays

Cells expressing the GPCR of interest can be transiently or stably transfected with a reporter gene plasmid construct containing an enhancer element which responds to activation of a second messenger signaling pathway or pathways, thereby controlling transcription of a cDNA encoding a detectable reporter protein. GPCR expression can be the result of endogenous expression on a cell line or cell type or the result of stable or transient transfection of DNA encoding the receptor of interest into a cell line by means commonly, used in the art. Immortalized cell lines or primary cell cultures can be used.

If the activated pathway is stimulatory (e.g., Gs or Gq), agonist activity results in activation of transcription factors, in turn causing an increase in reporter gene transcription, detectable by an increase in reporter activity. To test for agonist or inverse agonist activity, cells expressing the GPCR and the reporter gene construct can be challenged by the test compound for a predetermined period of time (e.g., 2-12 hours, typically 4 hours). Cells can then be assessed for levels of reporter gene product. Inverse agonists will suppress levels of reporter to below basal levels in a dose dependent manner. To test for antagonist or inhibitory activity through a stimulatory pathway, cells expressing both the GPCR and the reporter gene construct can be activated by a receptor agonist to increase gene reporter product levels. Treatment with antagonists will counter the effect of agonist stimulation in a dose- and receptor-dependent manner.

To test for agonist activity on receptor signaling through an inhibitory pathway (e.g., Gi, which couples to CXCR5), cells can be treated with a systematic activator (e.g., forskolin) to increase levels of reporter gene product. Activation of Gi by treatment with receptor agonist will inhibit this expression by inhibiting adenylyl cyclase. To screen for antagonist activity, test compounds can be assessed for the ability to counter agonist inhibition of adenylyl cyclase, resulting in increase reporter transcription.

Alternatively, a plasmid construct expressing the promiscuous G-protein Ga16 can be used to obtain a positive signal from a GPCR which normally couples to an inhibitory G-protein. Co-expression of the chimeric G-protein Gaq/Gai5 (Coward et al. Analytical Biochemistry 270, 242-248 (1999)) allows coupling to Gi-coupled receptors and conversion of second messenger signaling from the inhibitory Gi pathway to the stimulatory Gq pathway. Agonist and antagonist assessment in these systems is the same as the stimulatory pathways. Well-to-well variation caused by such factors as transfection efficiency, unequal plating of cells, and cell survival rates can be normalized in transient transfection assays by co-transfecting a constitutively expressing reporter gene with a non-interfering signal independent of the regulated reporter.

### Chemotaxis Assay

Chemotaxis assays were utilized to determine the effect of compound on the directed migration of cells in response to chemokine. 300.19 cells that express CXCR5 were placed in the upper chamber of a Transwell chemotaxis plate (Corning) and allowed to migrate through a polycarbonate membrane to a lower chamber containing the appropriate receptor-specific ligand. To test for antagonist or potentiating activity, cells were mixed with the desired concentration of compound prior to addition to the upper chamber. Conversely, agonist activity was determined by adding compound in the bottom chamber only without endogenous chemokine. The effect of compound is quantified by several parameters, including the extent of maximum response, the shift of agonist dose-response curves, and the area under the curve.

To measure the CXCR5-dependent migration of cells, the appropriate concentration of CXCL13 or test compound was diluted in phenol red-free RMPI-1640/20 mM HEPES/0.5% BSA buffer and placed in the bottom chamber of a transwell apparatus. 300.19 cells, a mouse pre-B cell line stably overexpressing CXCR5, were washed twice in buffer and resuspended at 130,000 cells/ml. A 75 μl sample of this suspension was mixed with the test compound of interest and placed in the upper chamber of a 5-micron transwell apparatus.

To initiate cell migration, the assembled transwell plate was placed in a 37° C., 0.5% CO₂ incubator for a specified time interval, typically between 30 and 120 minutes. After incubation, the unit was disassembled and the lower chamber placed at −80° C. overnight to facilitate lysis of cells. To quantify migrated cells, plates were thawed at 37° C. in a humidified chamber, and then a sample volume was removed from each well and mixed with an equal volume of CyQuant (Invitrogen) working solution in opaque plates. The fluorescence intensity of each well represents the DNA content and is directly proportional to cell number. Each sample was typically run in duplicate or triplicate and each plate included two separate negative controls. The plate background control, which included no cells in the upper chamber, was subtracted from all values. The negative control had no agonist added in the lower chamber, and served to establish the baseline for random migration.

The CXCR5 GPCR mediates the directed migration of cells in response to a gradient of its cognate ligand, CXCL13. The in vitro chemotaxis assay employed herein is designed to quantify the extent of cell movement across a porous membrane, which can be measured in the absence or presence of test compound. In general, chemokines produce bell-shaped ligand dose-response chemotaxis curves indicative of homologous desensitization at high ligand concentrations. A leftward shift of the dose-response bell curve in the presence of compound may be interpreted as a positive modulation activity. Conversely, a rightward shift of the dose-response bell-curve may be indicative of inhibition and/or negative modulation activity. Area under the curve (AUC) is also employed to quantify the overall height of the bell-shaped dose-response curve. An increase in the AUC in the presence of compound may be interpreted as positive modulation.

For example, cells incubated in the presence of the CXCR 5 compound 14 produced a leftward shift of the bell-shaped ligand dose-response curve and an increase in AUC compared to the vehicle control. Thus, the likely interpretation of the mode of action of this compound is positive modulation.

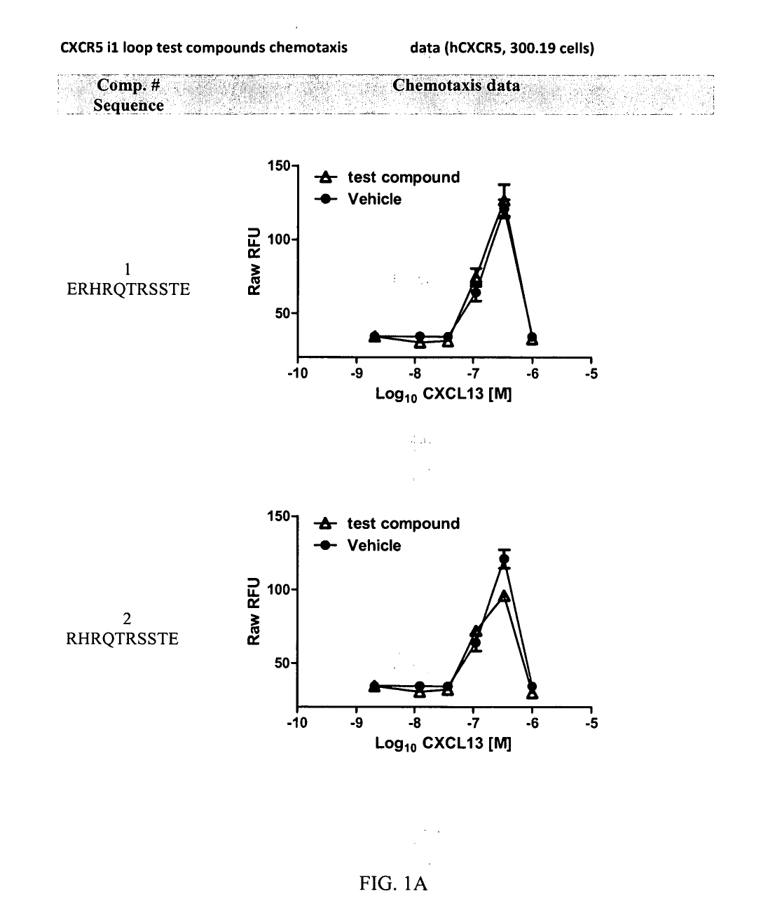

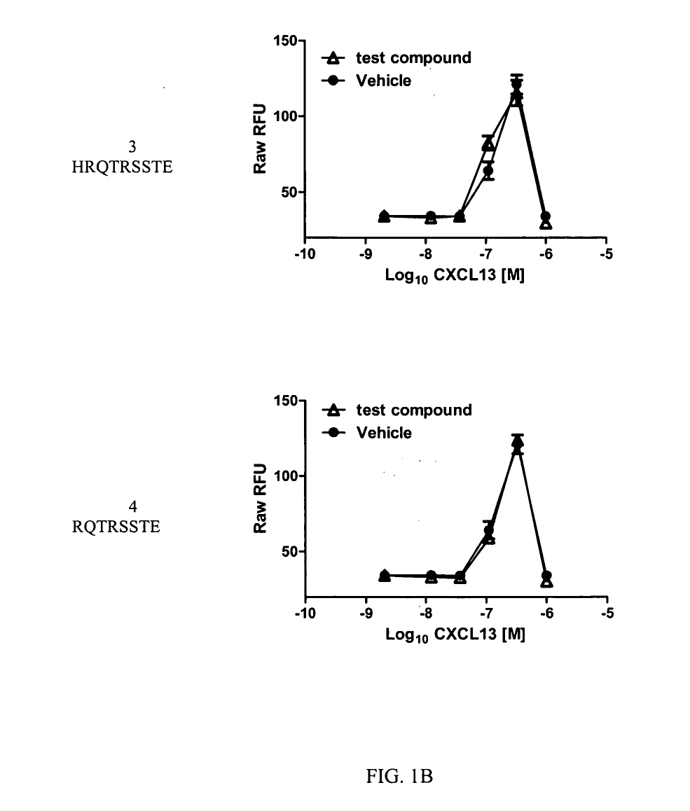

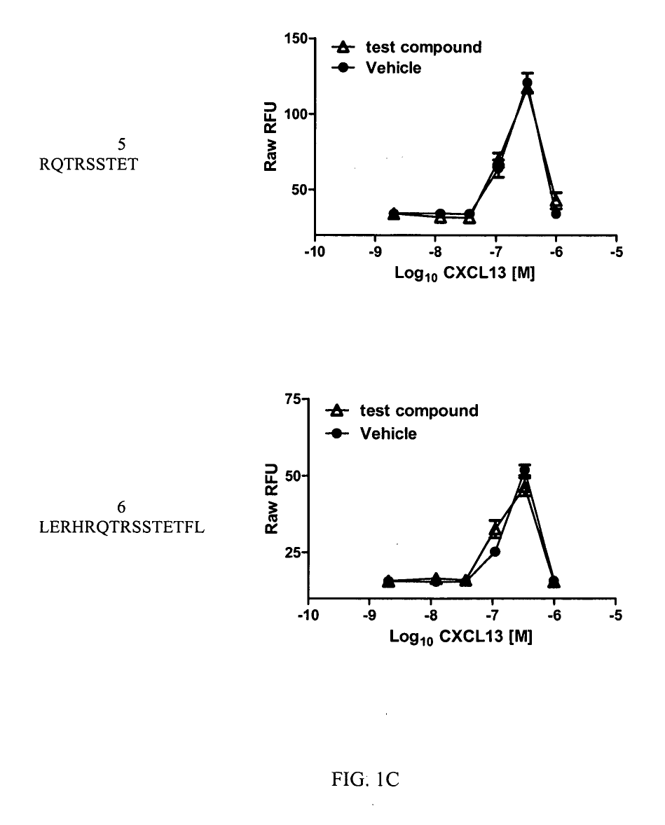

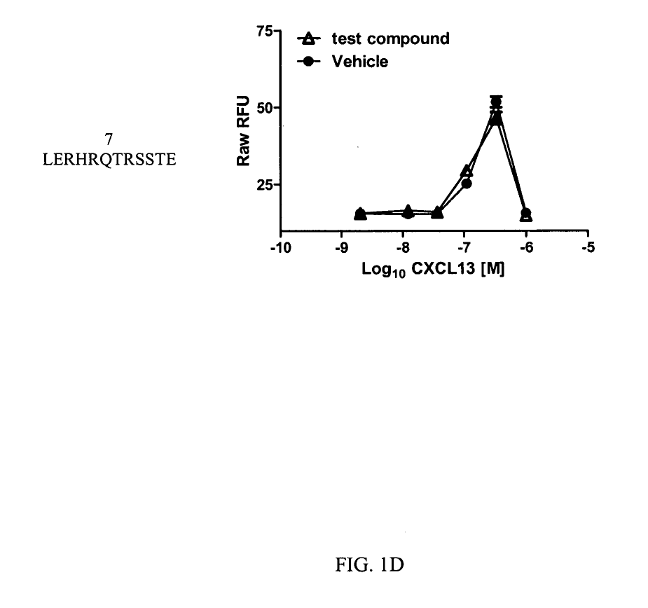

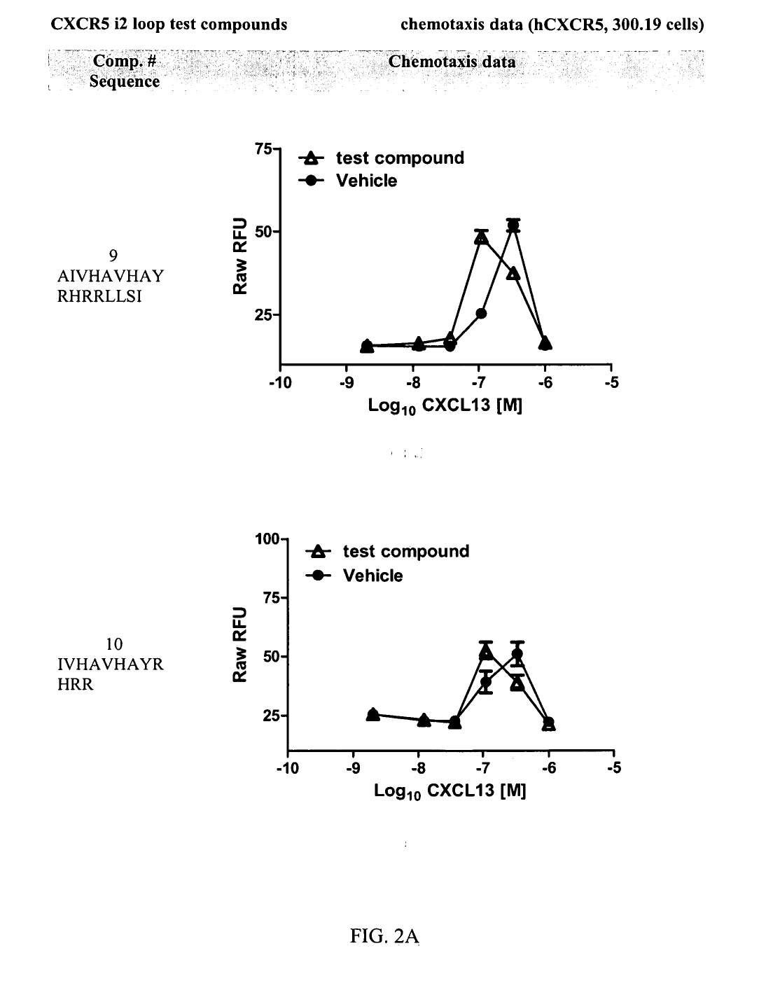

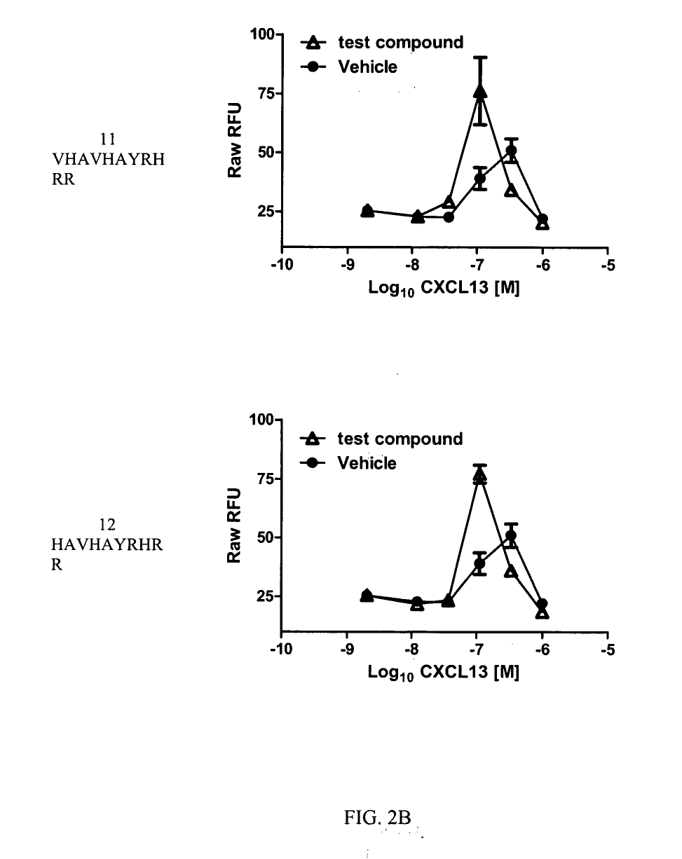

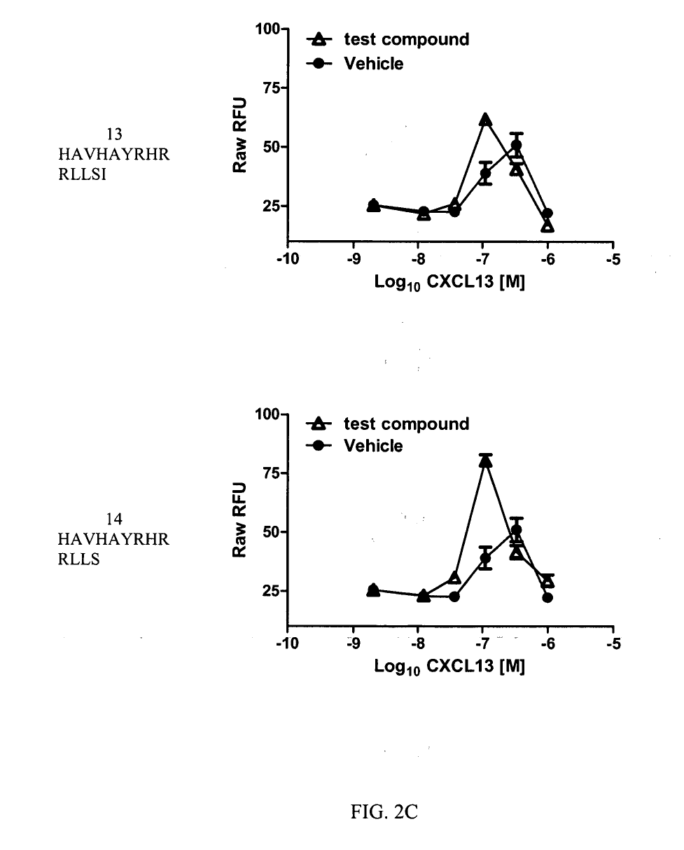

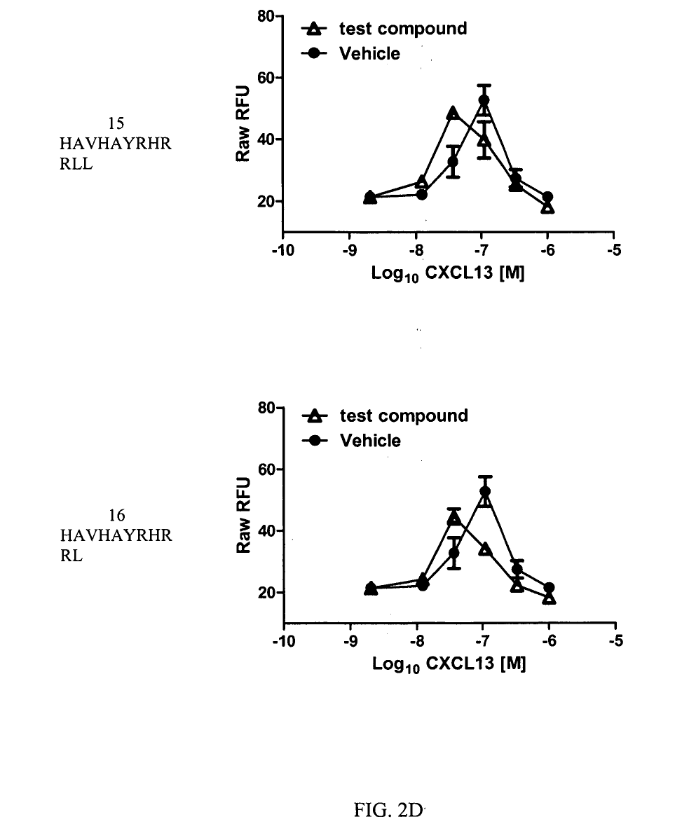

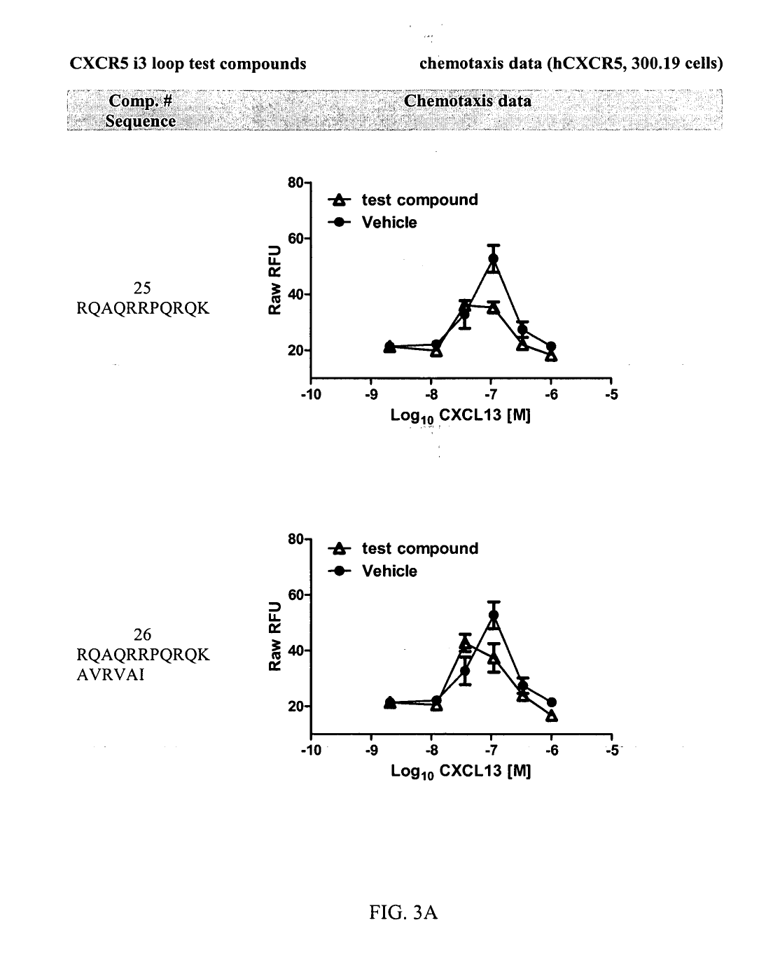

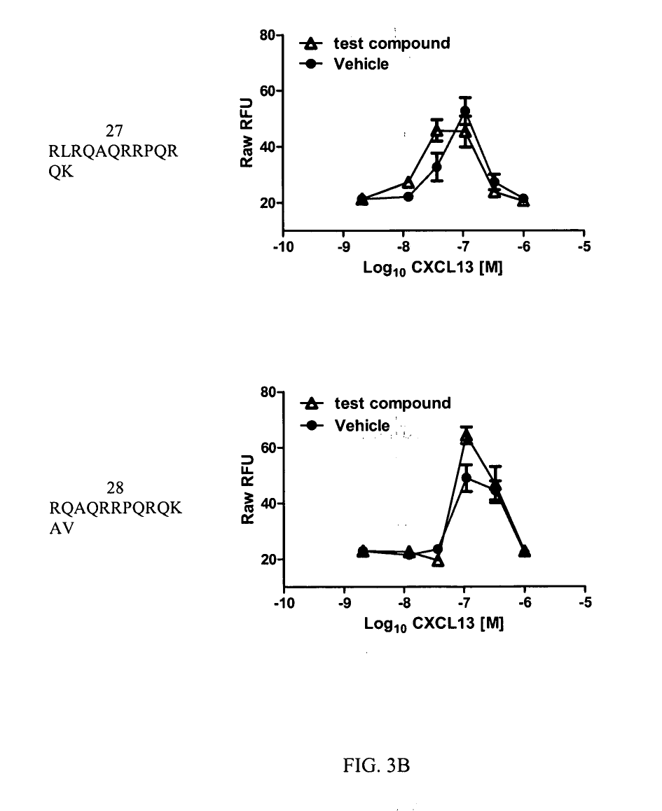

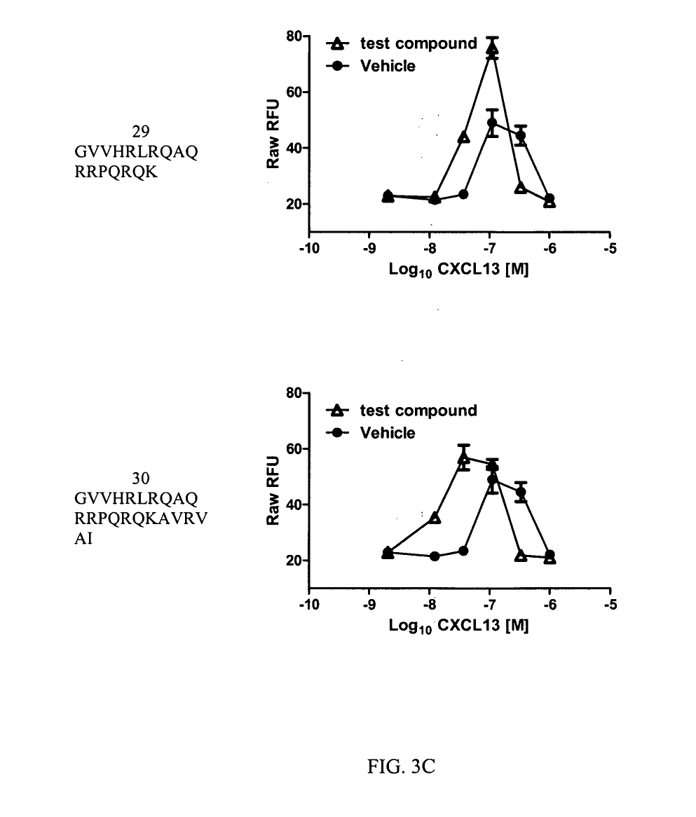

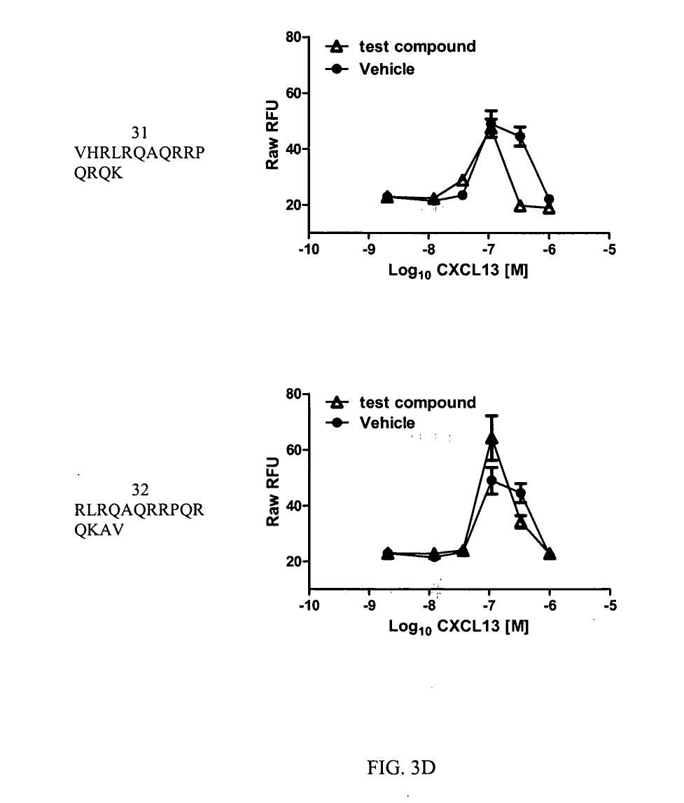

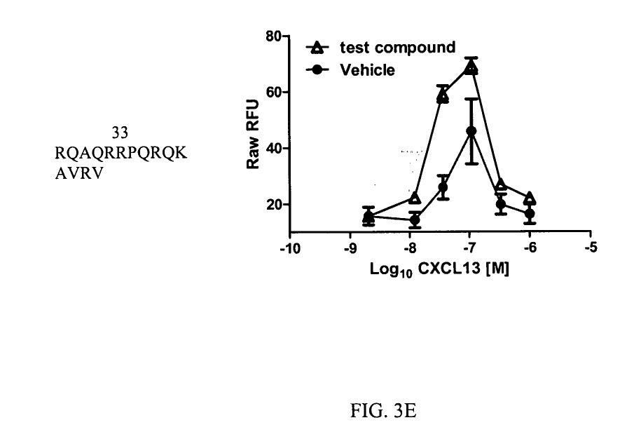

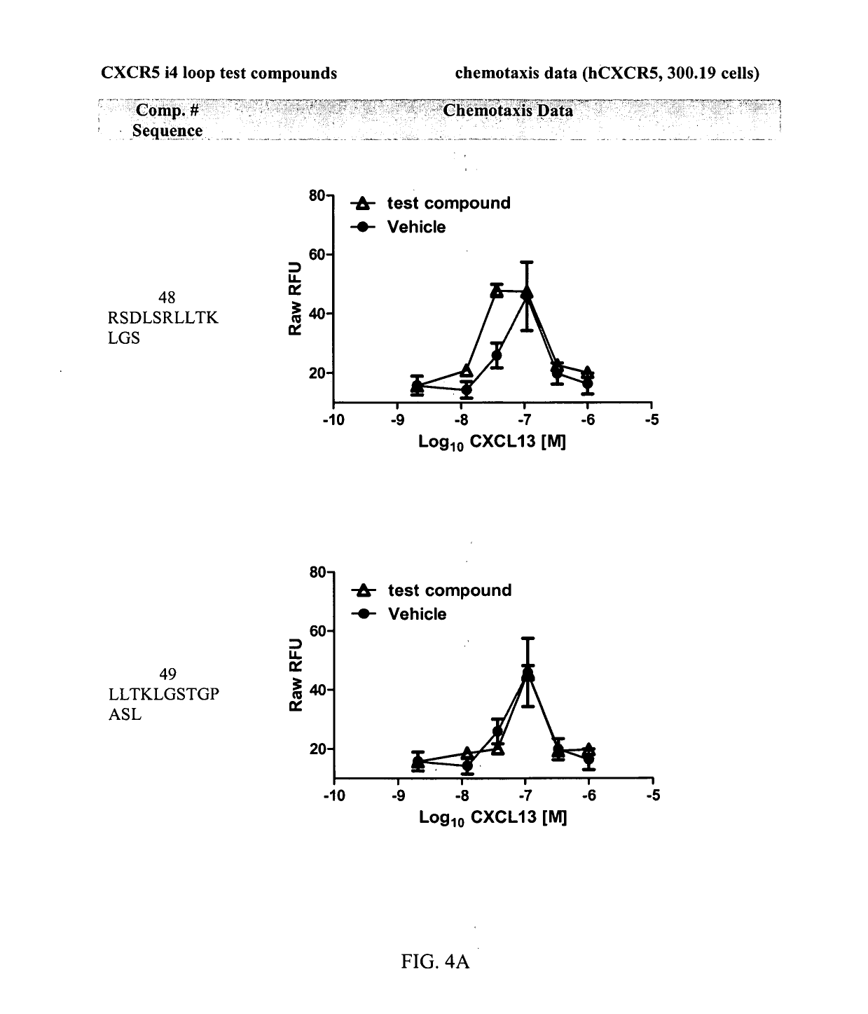

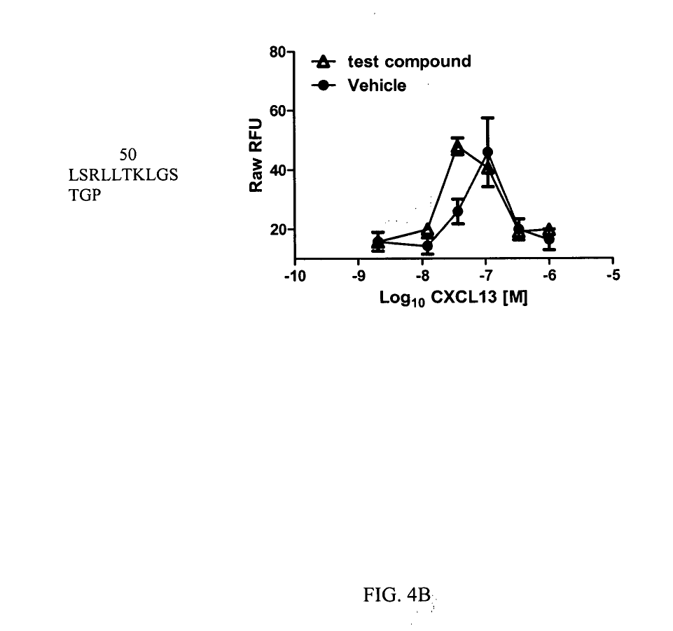

See FIGS. 1-4 for results.

### Calcium Flux Assay

The calcium flux assay is one of the most popular cell-based GPCR functional assays. It most often uses calcium sensing fluorescent dyes such as fura2 AM, fluo-4 and Calcium-4 to measure changes in intracellular calcium concentration. It is used mainly to detect GPCR signaling via the Gaq subunit. Activation of these Gq-coupled GPCRs leads to activation of phospholipase C, which subsequently leads to increase in inositol phosphate production. IP3 receptors on endoplasmic reticulum sense the change then release calcium into cytoplasm. Intracellular calcium binding to the fluorescent dyes can be detected by instruments that quantify fluorescent intensities, such as FLIPR Tetra, Flexstation (MDS) and FDSS (Hamamatsu). In addition to assessing Gq-coupled receptor signaling, calcium flux assays can also be used to study Gs and Gi coupled receptors by co-expressing CNG (cycic nucleotide gated calcium channel) or chimeric G-proteins (Gqi5, Gsi5 for example). Activation of some Gi-coupled receptors can also be detected by calcium flux assays via Gβγ mediated phospholipase C activation.

### CXCR5 Testing

An example of the calcium flux assay can be assessing CXCL13 activation of CXCR5 in stably transfected murine pre-B cells 300-19 cells cell line. Cells can be seeded into 96-well black plates with clear bottom at 200K/well in Hank's balanced salt solution with 20 mM HEPES, 0.1% BSA. After dye loading by incubating cells in Calcium-4 dye at room temperature for 1 hour, plates can be placed in aFlexstation 3 reader. The addition of test compound or reference antagonists can be done either by manual pipetting or by liquid handling on the Flexstation. The latter allows the assessment of agonist activity of the test compound. After incubation for 15 minutes at 37° C., CXCL13 can be added on the Flexstation and receptor activation can be assessed by measuring changes in fluorescent intensity.

### β-Arrestin Assay

The β-arrestin assays developed by DiscoverX employed herein detect binding of a ligand to the GPCR by directly measuring β-arrestin recruitment to the GPCR independent of G-protein coupling. In this system, β-arrestin is fused to an N-terminal deletion mutant of β-gal (termed the enzyme acceptor of EA) and the GPCR of interest is fused to a smaller (42 amino acids), weakly complementing fragment. In cells that stably express these fusion proteins, ligand stimulation results in the interaction of β-arrestin and the tagged GPCR, forcing the complementation of the two β-gal fragments and resulting in the formation of a functional enzyme that converts substrate to detectable signal. In practice, eXpress β-arrestin cells expressing the receptor of interest are incubated with either compound alone (agonist mode), or compound plus native ligand (allosteric mode). A chemiluminescent substrate is then added to determine the extent of enzyme activity, and by inference, arrestin recruitment to GPCR.

### Results

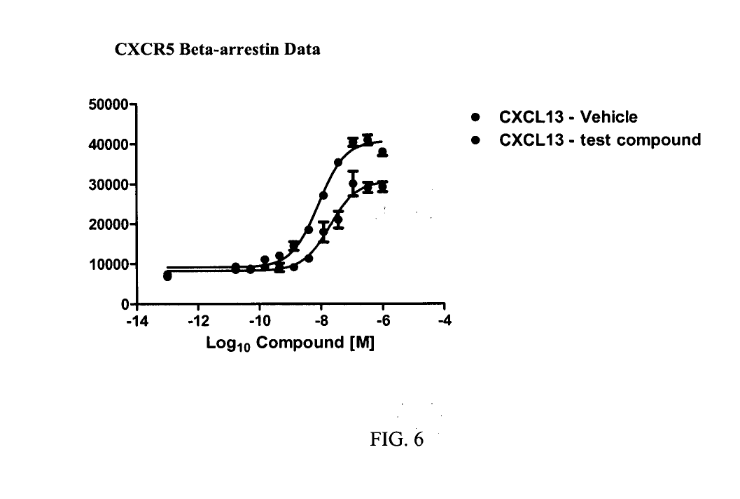

The β-arrestin assays for CXCR5 Compound 14 were run in both agonist and allosteric modes. In agonist mode, the CXCR5-expressing eXpress β-arrestin cells were incubated with a dose curve of CXCR5 Compound 14 in the absence of CXCL13. In this modality, any increase in GPCR activity is interpreted as agonism. The lack of response in this mode suggests that CXCR5 Compound 14 does not have agonist activity. In allosteric mode, Compound 14 is co-incubated with CXCL13, and the CXCR5 response of the eXpress cells is quantified. In this configuration, the addition of Compound 14 produced a left-shifted and higher maximal response curve, indicative of positive modulation. See FIG. 6.

HTRF cAMP Assay and IP-One Assay (Cisbio)

HTRF (homogeneous time resolved fluorescence) is a technology developed by Cisbio Bioassays based on TR-FRET (time-resolved fluorescence resonance energy transfer). Cisbio Bioassays has developed a wide selection of HTRF-based assays compatible with whole cells, thereby enabling functional assays run under more physiological conditions. cAMP kits are based on a competitive immunoassay using cryptate-labeled anti-cAMP antibody and d2-labeled cAMP. This assay allows the measurement of increase in intracellular cAMP upon Gs-coupled receptor activation as well as decrease in forskolin stimulated increase in cAMP upon Gi-coupled receptor activation. The IP-One assays are competitive immunoassays that use cryptate-labeled anti-IP1 monoclonal antibody and d2-labeled IP1. IP1 is a relatively stable downstream metabolite of IP3, and accumulates in cells following Gq receptor activation.

### Results

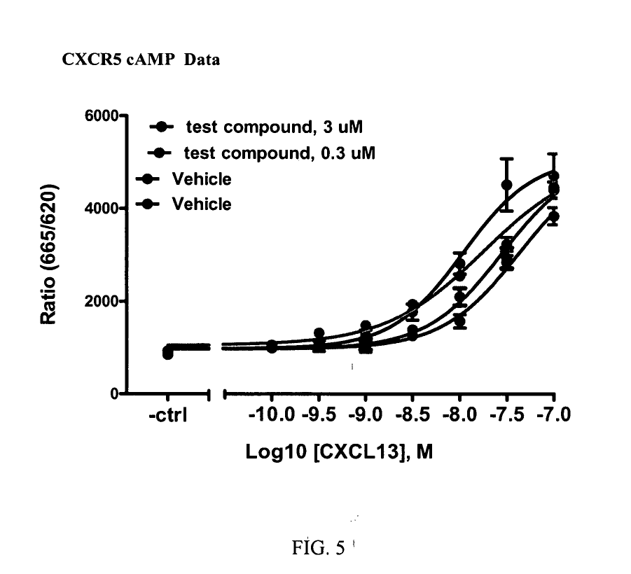

A cAMP assay was performed in the presence of Compound 14 to assess the activation of CXCR5 by the production of cAMP. HEK293 cells expressing hCXCR5 were incubated with CXCL13, and the inhibition of forskolin-stimulated cAMP production was quantified in cells in the absence and presence of Compound 14. Cells that were incubated with 2 different doses of Compound 14 (3 uM, 0.3 uM) produced cAMP responses that were shifted leftward in a dose-dependent manner. This response is indicative of a reduction of decrease in forskolin-stimulated cAMP due to enhanced CXCR5 (Gi) activation. This data suggests that Compound 14 acts as a positive modulator for CXCR5. See FIG. 5

### AlphaScreen Cellular Kinase Assays.

GPCR activation results in modulation of downstream kinase systems and is often used to probe GPCR function and regulation. TGR Bioscience and PerkinElmer have developed Surefire cellular kinase assay kits that are HTS capable and useful in screening kinase regulation. Such kits enable the monitoring of Gi regulated downstream kinases like ERK1/2. The assay allows the measurement of increases in ERK1/2 kinase phosphorylation upon Gi coupled receptor (e.g., CXCR5) activation and this signal in turn can be used to assay Gi coupled receptor modulator. Similar kits are also availibel to assay other pathway dependent signaling kinases such as MAP and BAD.

### In Vivo Assays

The G-protein coupled receptor CXCR5 is important in several therapeutic areas such as autoimmune diseases including Lupus, HIV and rheumatoid arthritis, chronic lymphocytic leukemia, tumor metastasis, and multiple sclerosis. CXCR5 receptor compounds of the present invention (agonists, antagonists, modulators) can be assessed using suitable in vivo models. Such in vivo models include mouse models of systemic lupus erythematosis (SLE) and mouse models of B-cell chronic lymohocytic leukemia. Further details regarding such models may be found in J. Exp. Med., June 2001: 193: 1393-1402.

The efficacy of CXCR5 receptor compounds of the invention on spleen tumor formation and liver foci formation liver intrasplenic tumor formation will be assayed using CT26 mouse colon carcinoma cells. Tumor CT26 (CRL-2639 from ATCC) CXCR5 expressing or CT26 (CRL-2639 from ATCC) control cells will be injected into the spleen (Meijer et al., 2006, Cancer Research, 66:9576-9582). Efficacy in spleen tumor formation and liver foci formation will be assessed as a function of CXCR5 derived compounds described herein. Animals will be sacrificed after day 14. Tumor burden will be analyzed using beta-galactosidase reporter gene.

In vivo assays for immunization will be performed by monitoring IgG production in an Immunization model. The effect of CXCR5 derived compounds described herein will be evaluated in mice in response to immunization delivered either by ip or in the footpads (tetanus toxin recommended; other options include SRBC, sheep erythrocytes, TNP-OVA etc). At the second immunization, a CXCR5 receptor compound having antagonist activity is administered and the effects on the IgG response measured by ELISA. These mice will be evaluated for serum IgG production as well as for T-cell B-cell ratios in peripheral blood, lymph nodes and spleen. A positive effect is suppression of the anti-tetanus IgG.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A compound represented by Formula A:
T-L-X₁-X₂-X₃-X₄-X₅-X₆-X₇-X₈-X₉-X₁₀-X₁₁-X₁₂-X₁₃-X₁₄-X₁₅-
X₁₆-X₁₇-X₁₈-X₁₉-X₂₀-R₁;
or a pharmaceutically acceptable salt thereof, wherein: L is a linking moiety represented by C(O) and bonded to the N terminal nitrogen of X₁ or the next present amino acid residue if X1 is not present; T is a lipophilic tether moiety bonded to L; and R₁ is OR₂ or N(R₂)₂, wherein each R₂ is independently H or alkyl, wherein at least three contiguous X₁-X₂₄ amino acid residues are present, and wherein:
X₁ is a leucine residue or absent,
X₂ is a valine residue or absent,
X₃ is isoleucine or absent,
X₄ is a leucine residue or absent,
X₅ is a glutamic acid residue or absent,
X₆ is a arginine residue or absent,
X₇ is a histidine residue or absent,
X₈ is a arginine residue,
X₉ is a glutamine residue,
X₁₀ is a threonine residue,
X₁₁ is a arginine residue,
X₁₂ is a serine residue,
X₁₃ is a serine residue,
X₁₄ is a threonine residue or absent,
X₁₅ is a glutamic acid residue or absent,
X₁₆ is a threonine residue or absent,
X₁₇ is a phenylalanine residue or absent,
X₁₈ is a leucine residue or absent,
X₁₉ is a phenylalanine residue or absent,
X₂₀ is a histidine residue or absent.

2. The compound of claim 1, wherein X₁₆-X₂₀ are absent and wherein
X₁₄ is a threonine residue, and
X₁₅ is a glutamic acid residue.

3. The compound of claim 1, wherein X₁-X₆, X₁₉ and X₂₀ are absent.

4. The compound of claim 1, wherein X₁-X₃ are absent.

5. The compound of claim 1, wherein:
X₁ is a leucine residue,
X₂ is a valine residue,
X₃ is isoleucine residue,
X₄ is a leucine residue,
X₅ is a glutamic acid residue,
X₆ is an arginine residue,
X₇ is a histidine residue,
X₁₄ is a threonine residue or absent,
X₁₅ is a glutamic acid residue or absent,
X₁₆ is a threonine residue or absent,
X₁₇ is a phenylalanine residue or absent,
X₁₈ is a leucine residue or absent,
X₁₉ is a phenylalanine residue or absent, and
X₂₀ is a histidine residue or absent.

6. The compound of claim 5, wherein X₁₄-X₂₀ is absent.

7. The compound of claim 1, selected from: 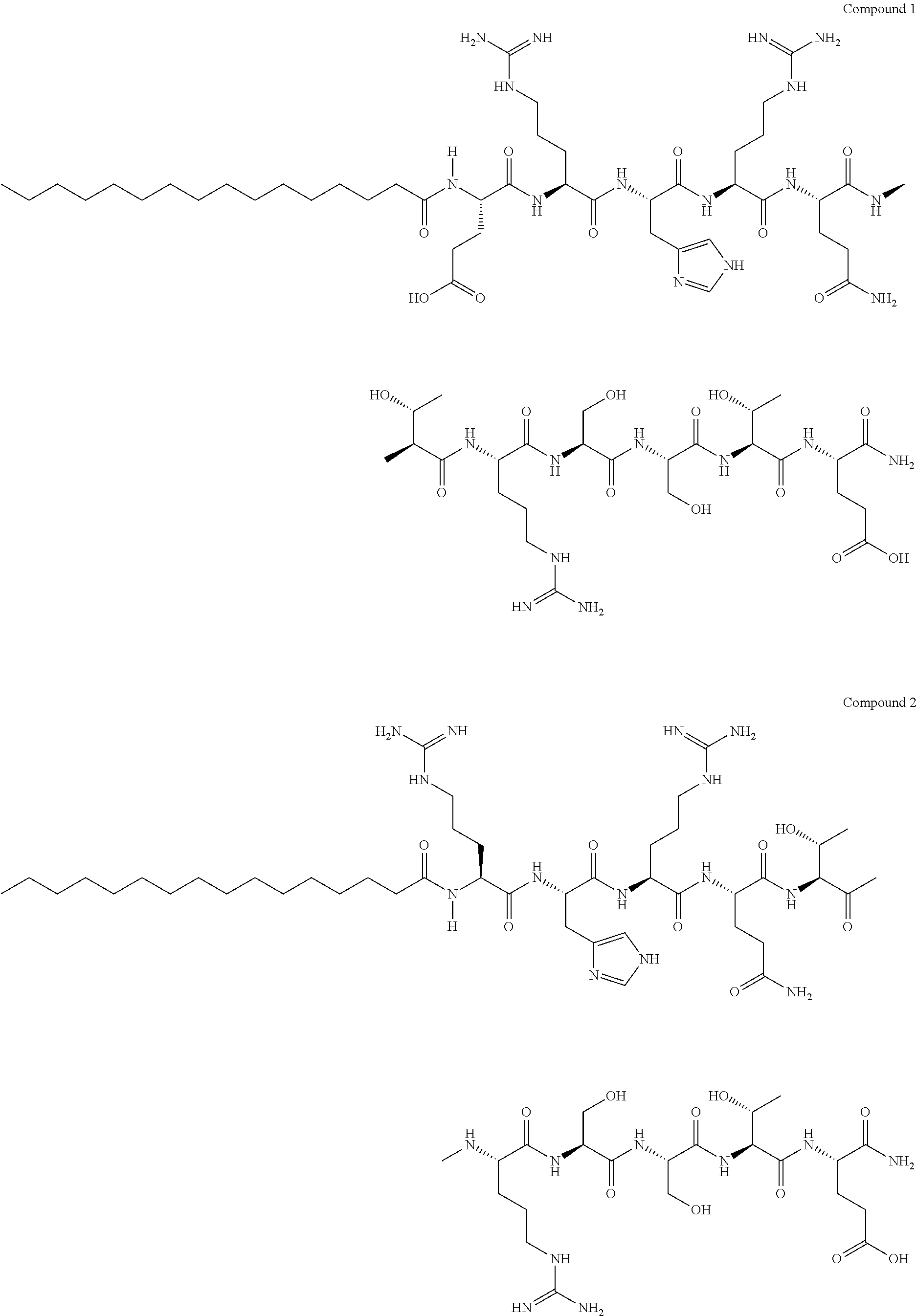 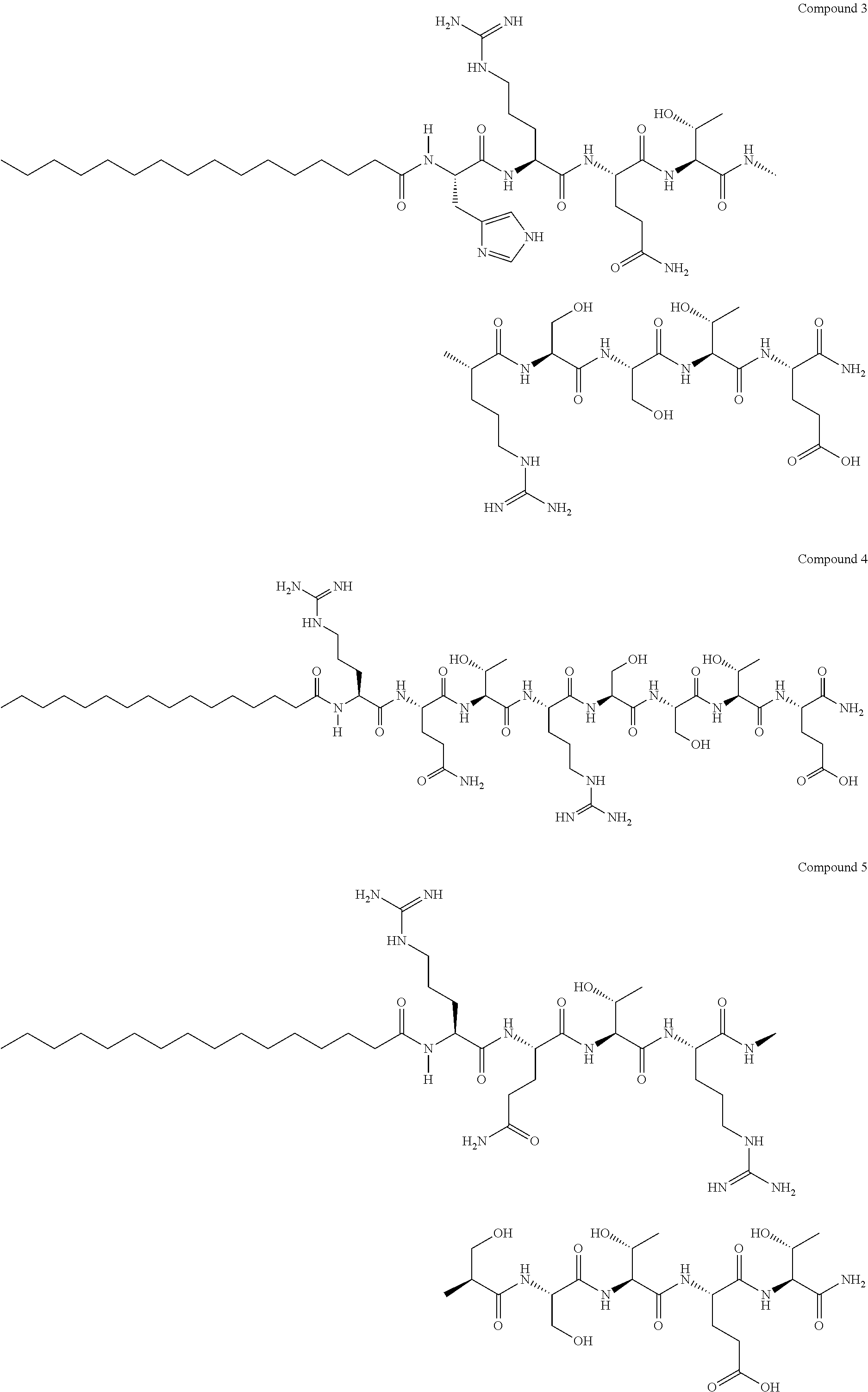 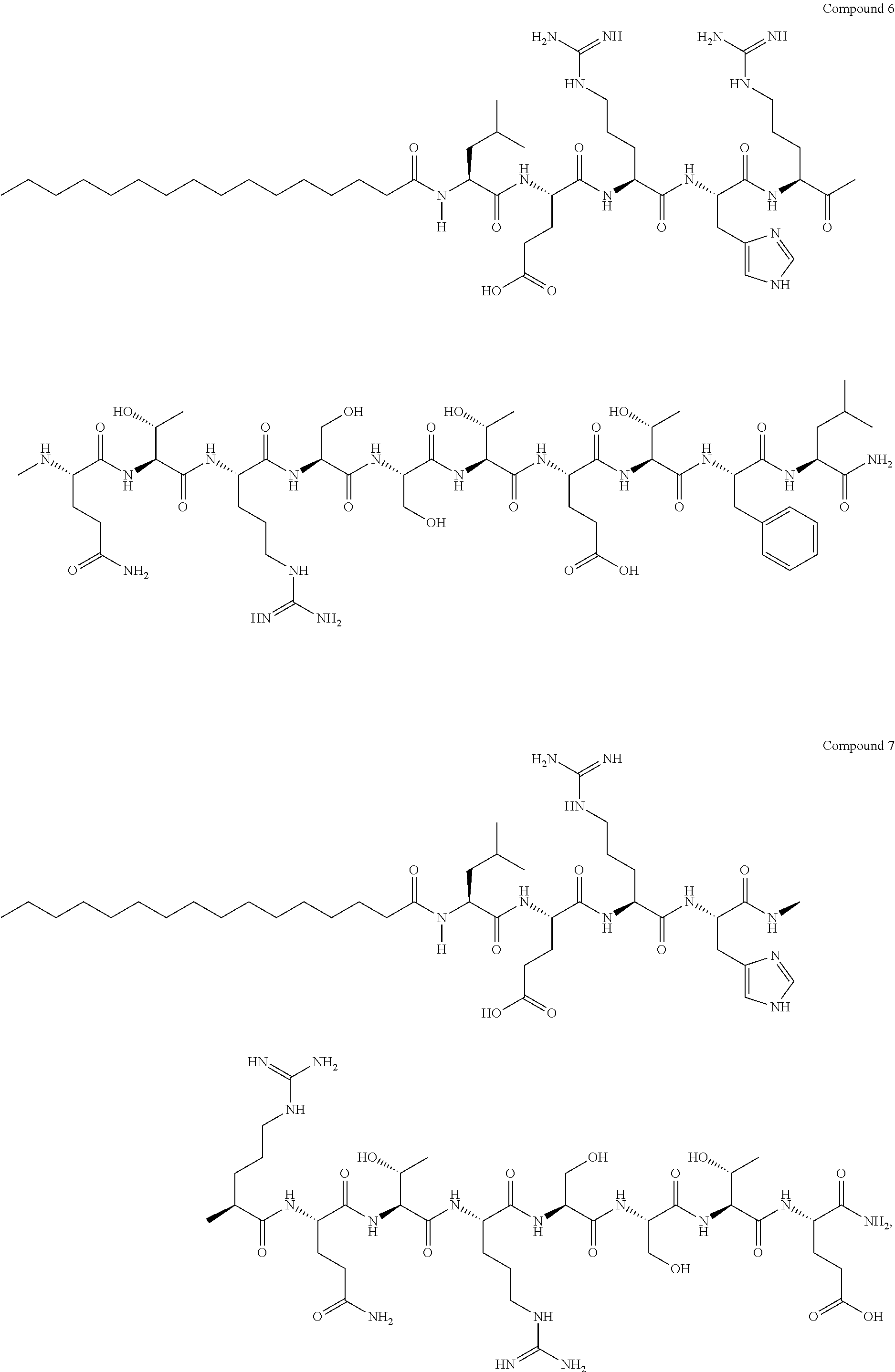 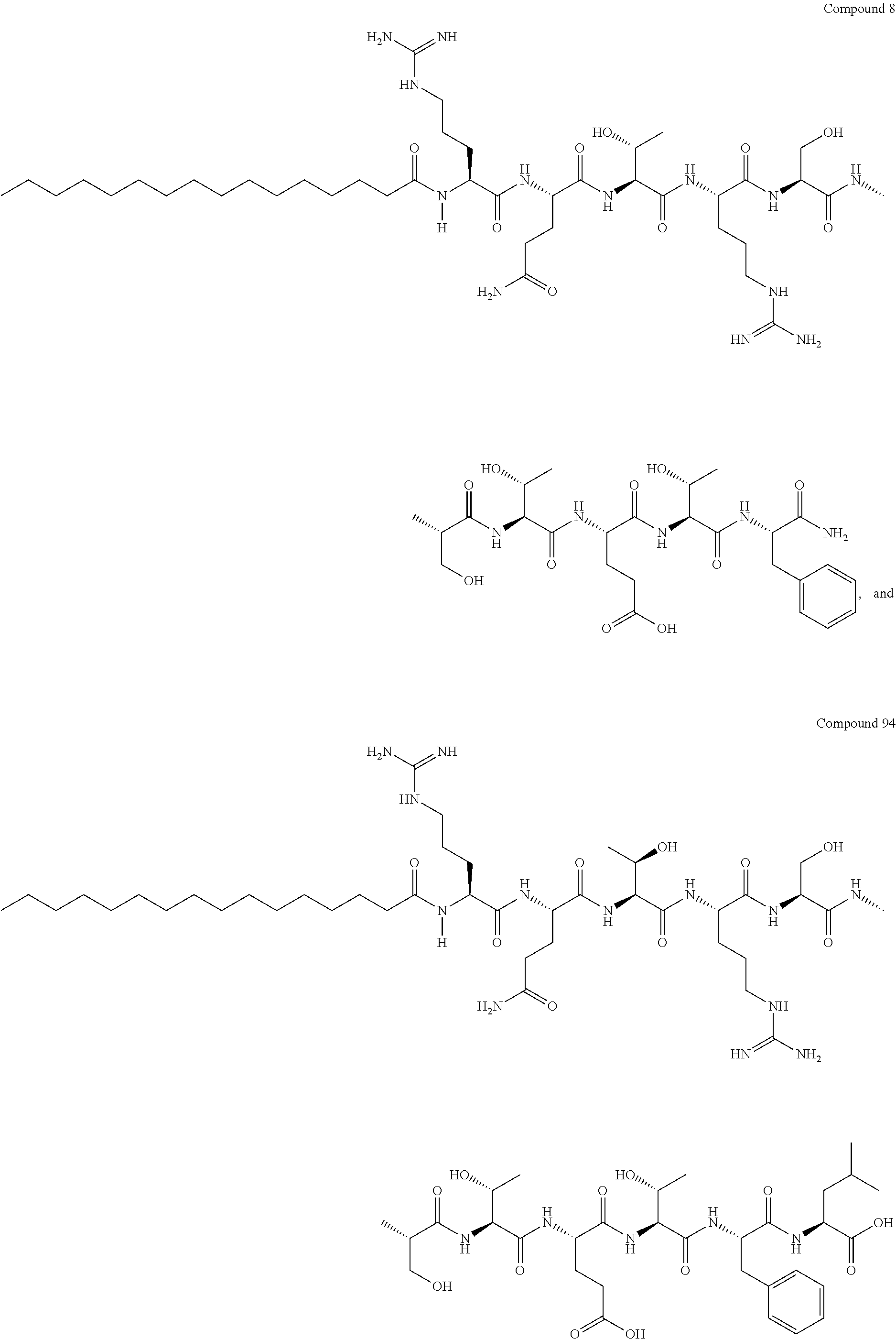
or a pharmaceutically acceptable salt of any of the foregoing compounds.

8. A compound represented by Formula B or a pharmaceutically acceptable salt thereof:
T-L-Y₁-Y₂-Y₃-Y₄-Y₅-Y₆-Y₇-Y₈-Y₉-Y₁₀-Y₁₁-Y₁₂-Y₁₃-Y₁₄-Y₁₅-
Y₁₆-Y₁₇-Y₁₈-Y₁₉-Y₂₀-Y₂₁-Y₂₂-Y₂₃-Y₂₄-Y₂₅-R1;
wherein L is a linking moiety represented by C(O) and bonded the N terminal nitrogen of Y₁ or the next present amino acid residue if Y₁ is absent, T is a lipophilic tether moiety bonded to L; and R₁ is OR₂ or N(R₂)₂, wherein each R₂ is independently H or alkyl, wherein at least three contiguous Y₁Y₂₅ amino acid residues are present, and wherein:
Yᵢ is a leucine residue or absent,
Y₂ is an alanine residue or absent,
Y₃ is an isoleucine residue or absent,
Y₄ is a valine residue or absent,
Y₅ is a histidine residue, alanine residue or absent,
Y₆ is an alanine residue or absent,
Y₇ is a valine residue or absent,
Y₈ is a histidine residue,
Y₉ is an alanine residue,
Y₁₀ is a tyrosine residue,
Y₁₁ is an arginine residue,
Y₁₂ is a histidine residue,
Y₁₃ is an arginine residue,
Y₁₄ is an arginine residue,
Yᵢₛ is a leucine residue or absent,
Y₁₆ is a leucine residue or absent,
Y₁₇ is a serine residue or absent,
Y₁₈ is an isoleucine residue or absent,
Y₁₉ is a histidine residue or absent,
Y₂₀ is an isoleucine residue or absent, and
Y₂₁ is a threonine or residue absent.

9. The compound of claim 8, wherein Y₁, Y₂, and Y₁₅-Y₂₁ are absent and Y₅ is histadine.

10. The compound of claim 8, wherein Y₁-Y₄ and Y₁₉-Y₂₁ are absent.

11. The compound of claim 10, wherein Y₅ is a histadine residue.

12. The compound of claim 10, wherein Y₅ is absent or an alanine residue.

13. The compound of claim 8, wherein Y₁-Y₂₁ are present.

14. The compound of claim 8, selected from: 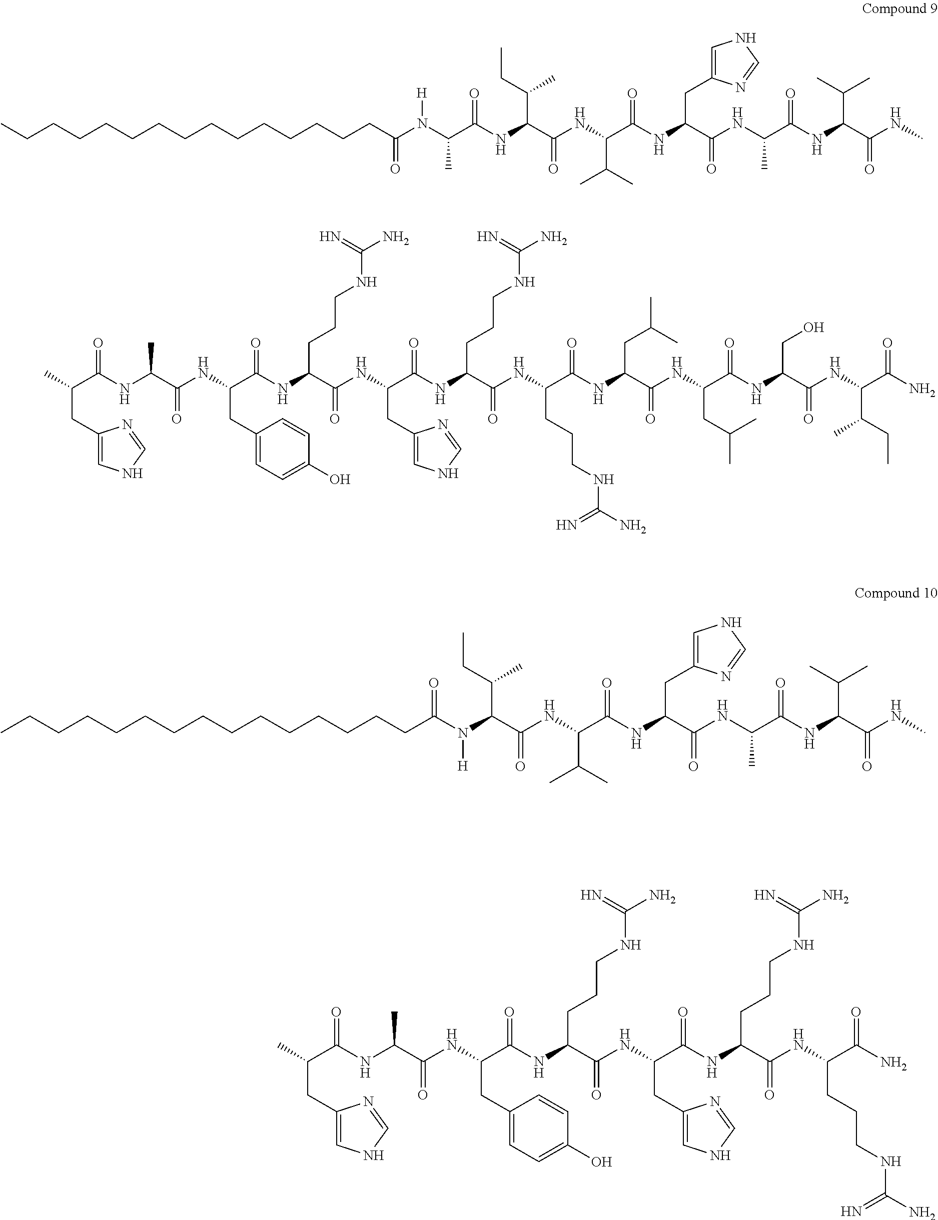 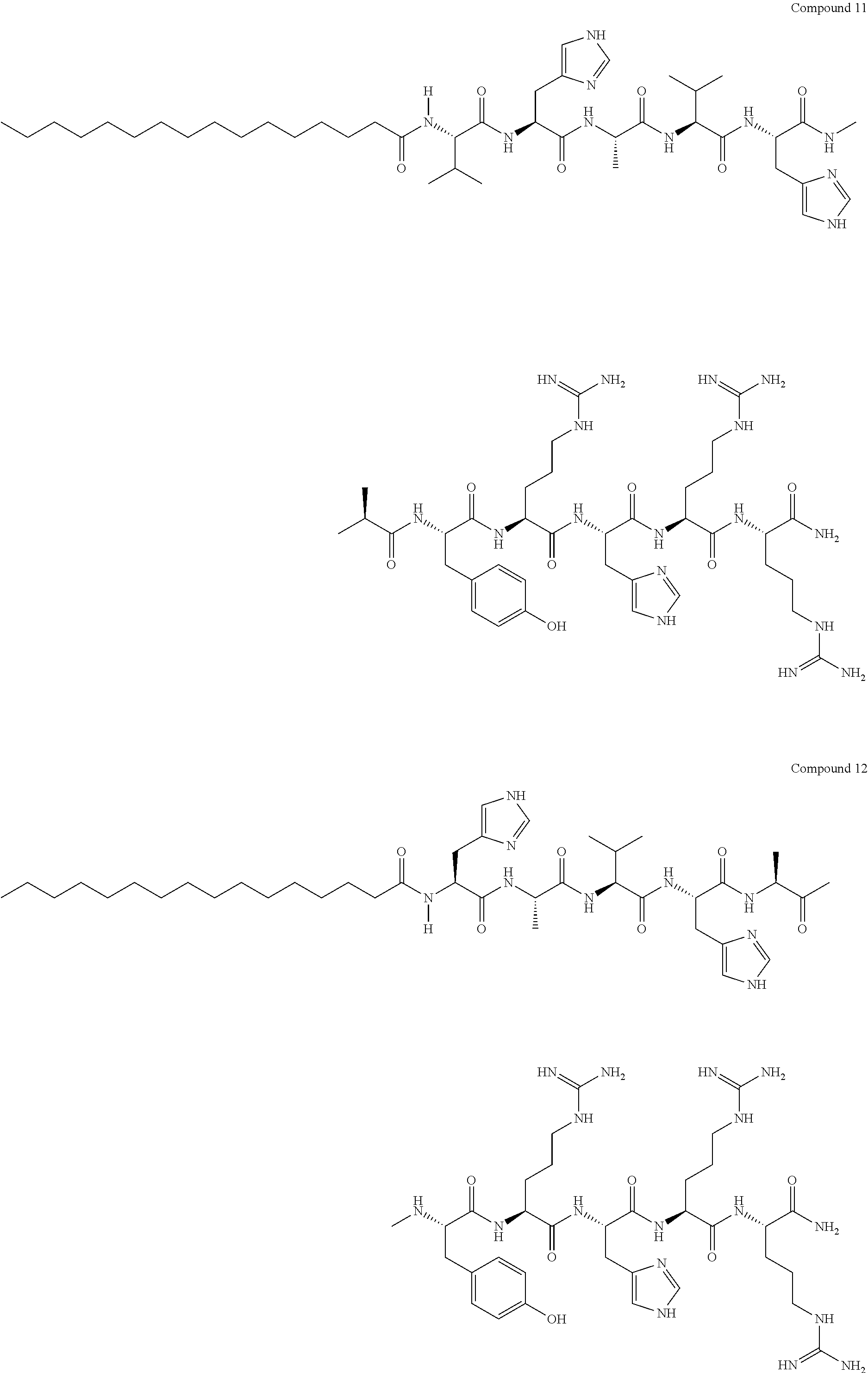 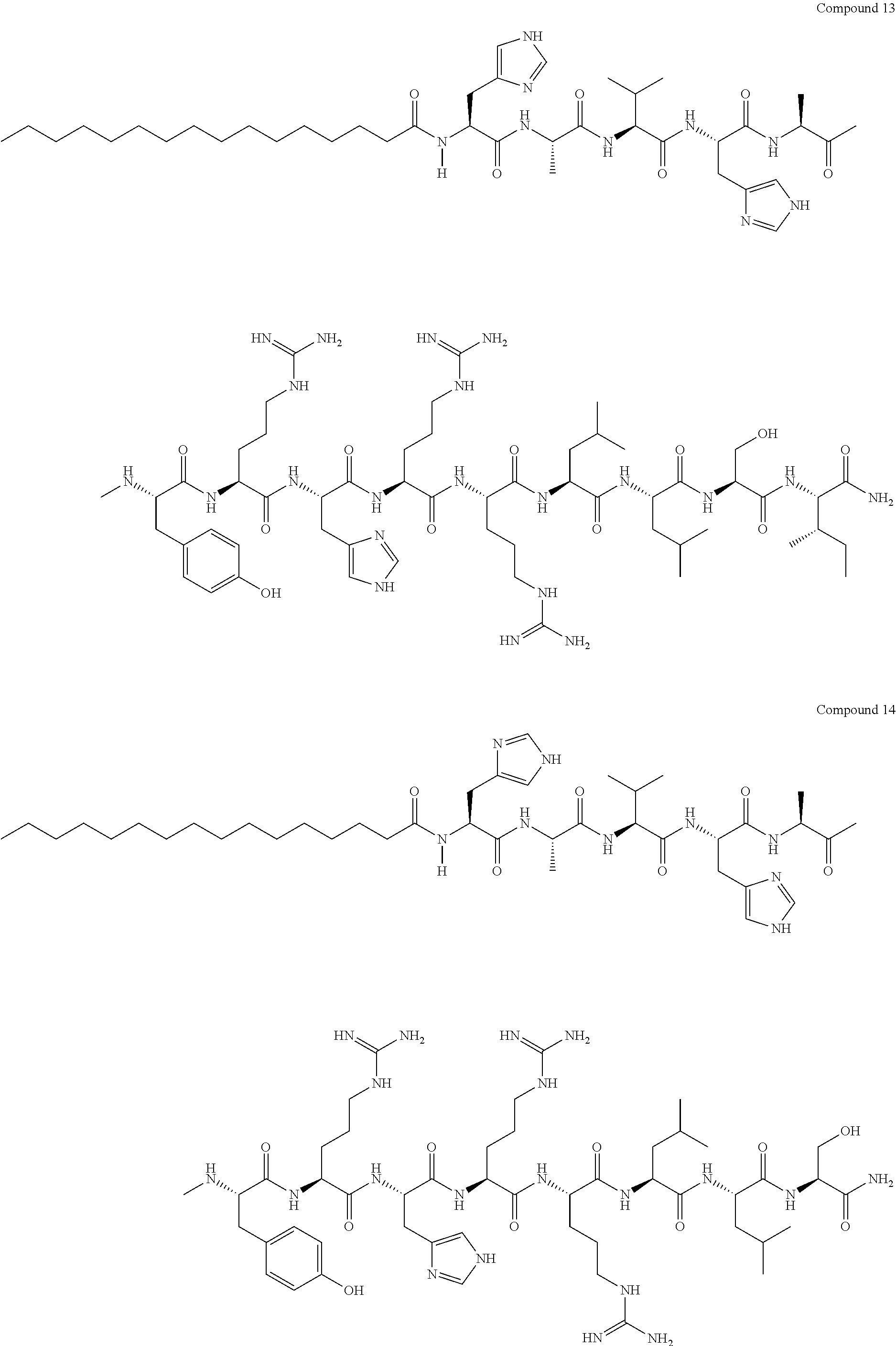 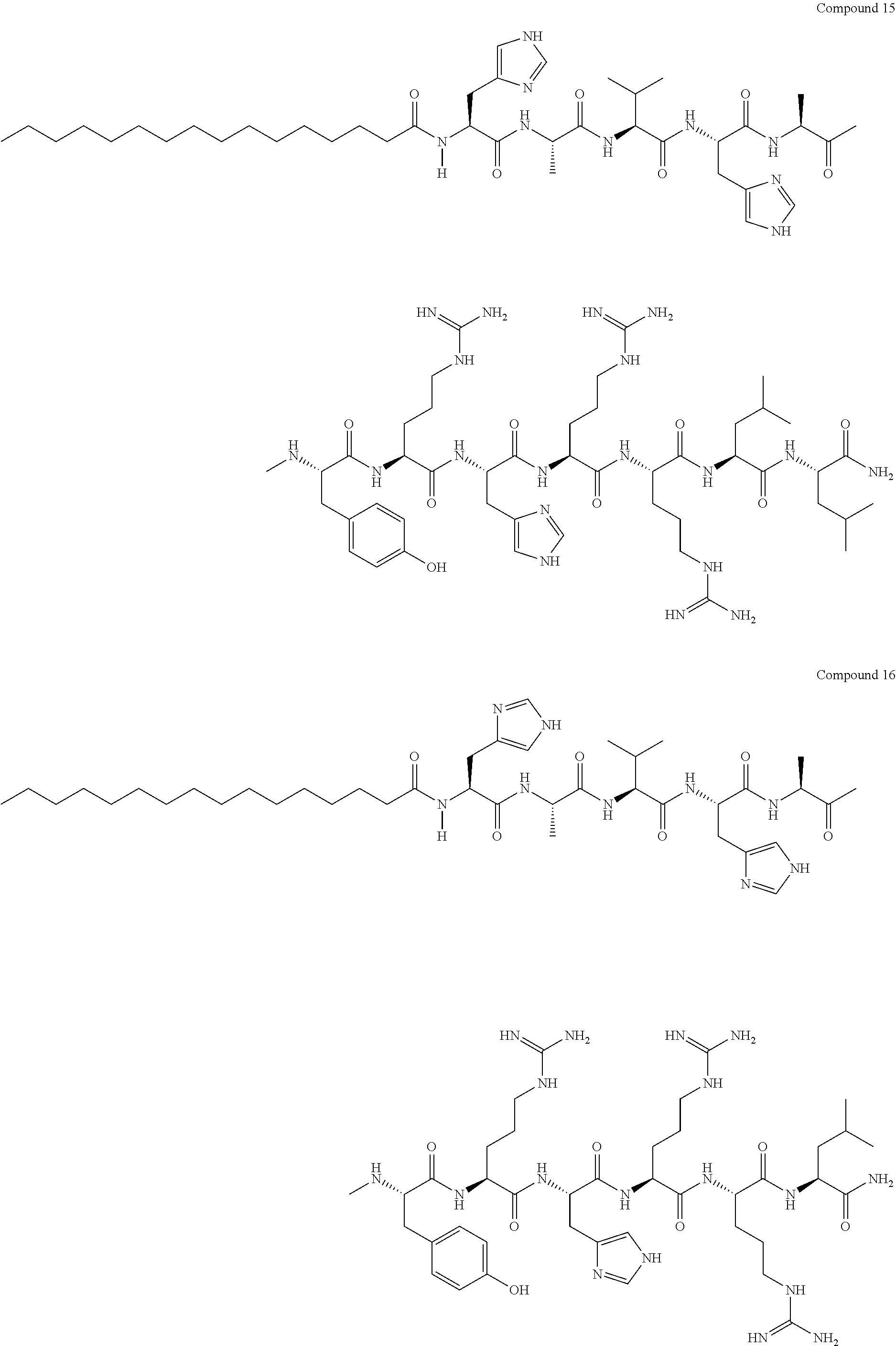 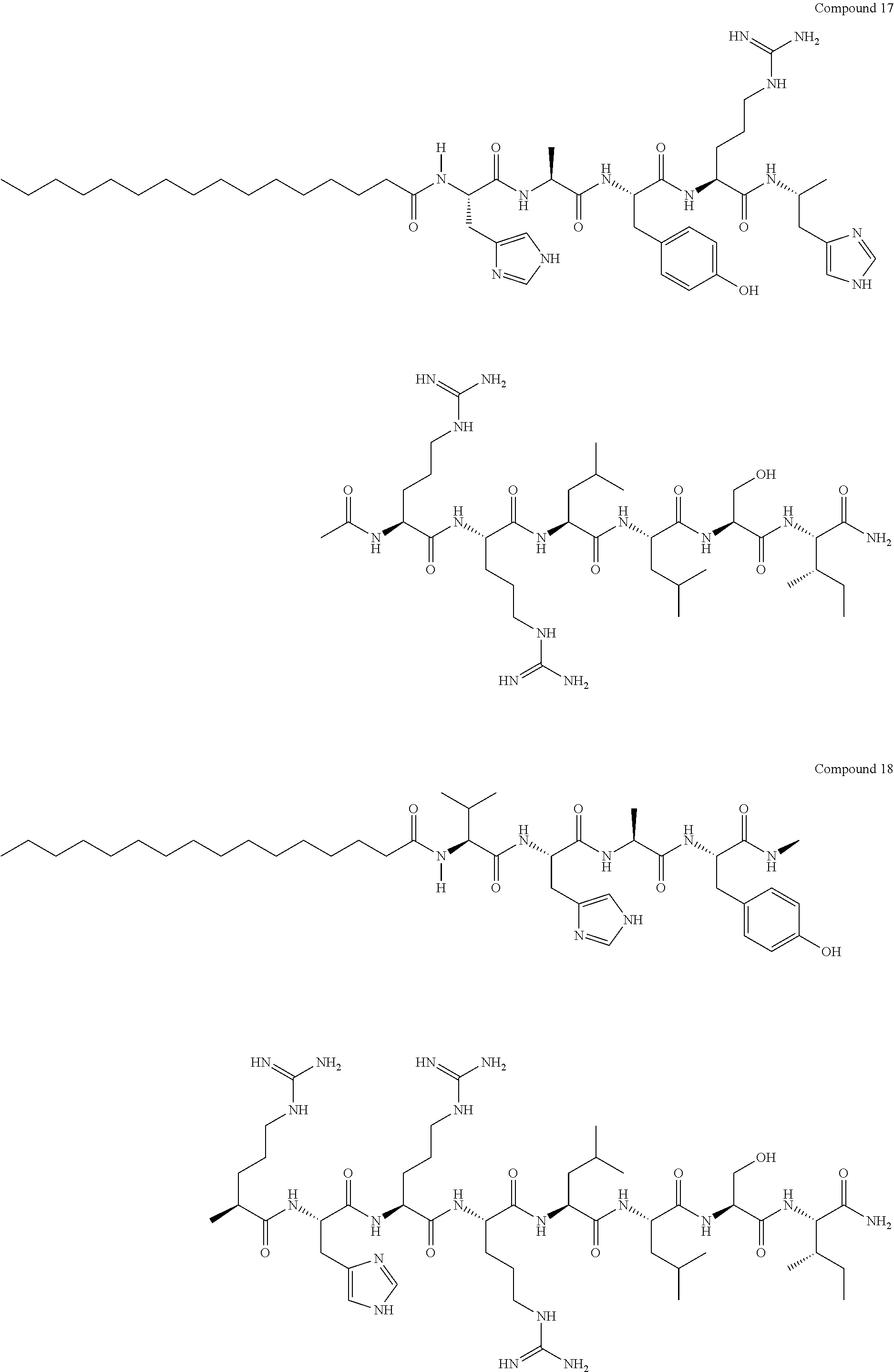 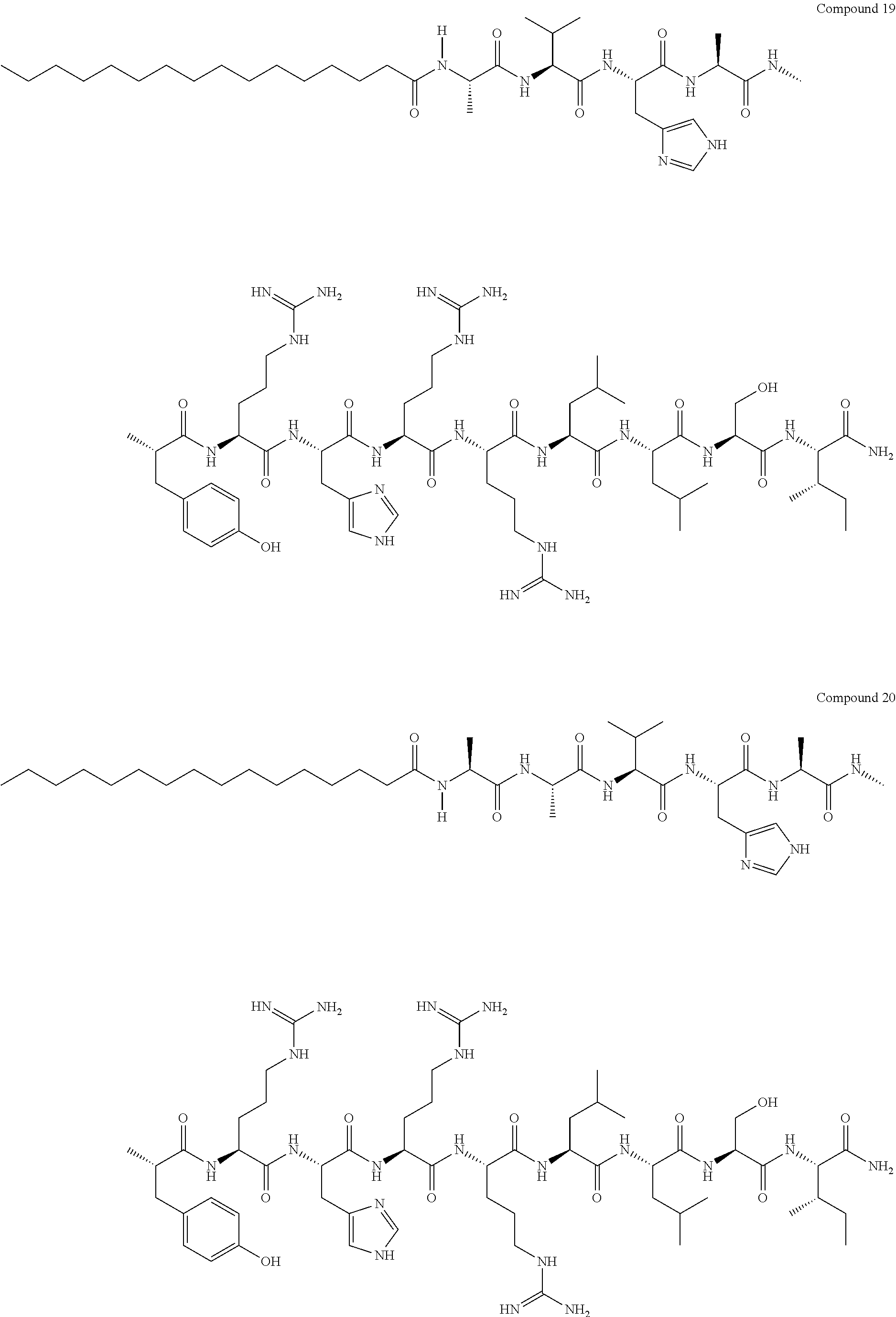 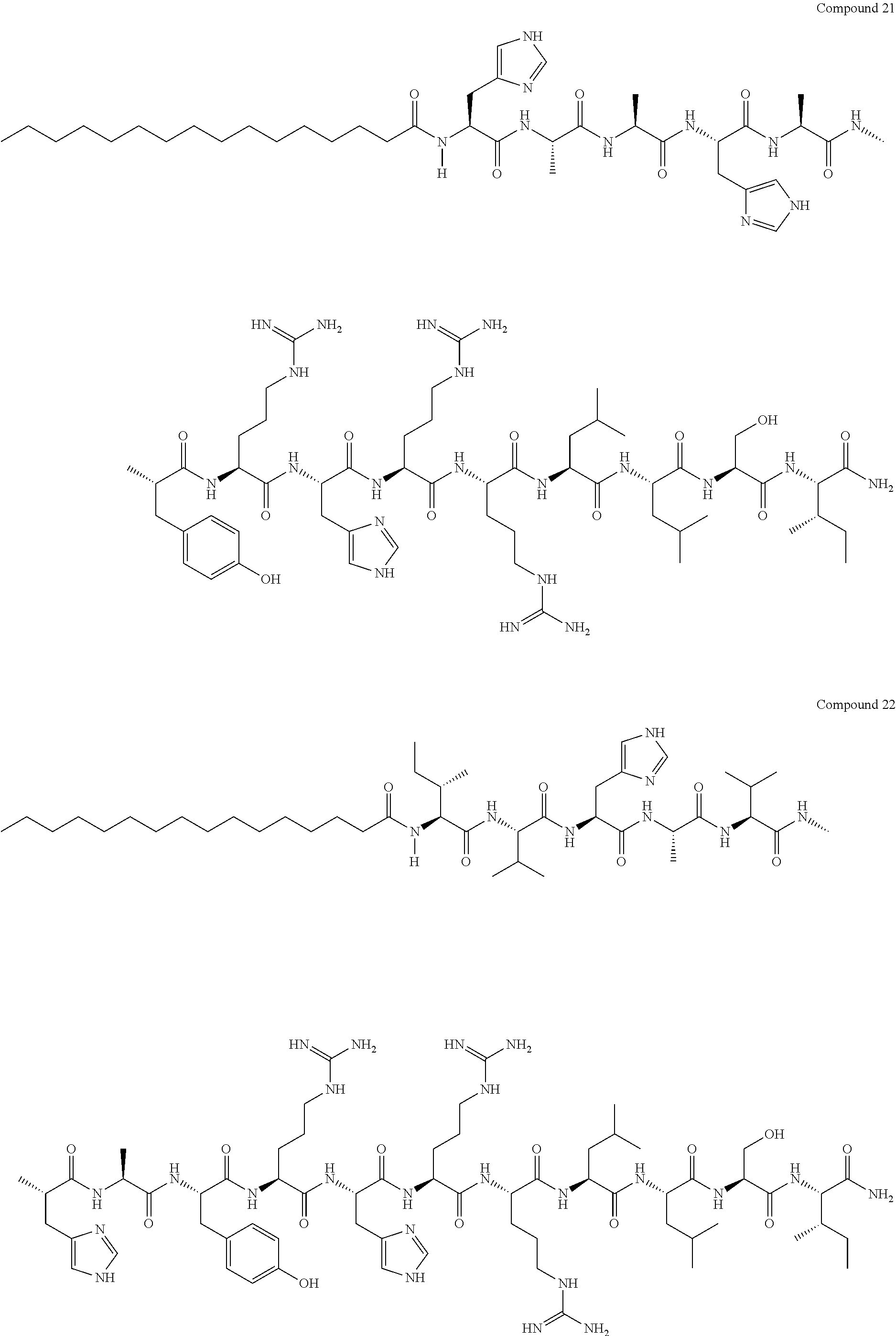 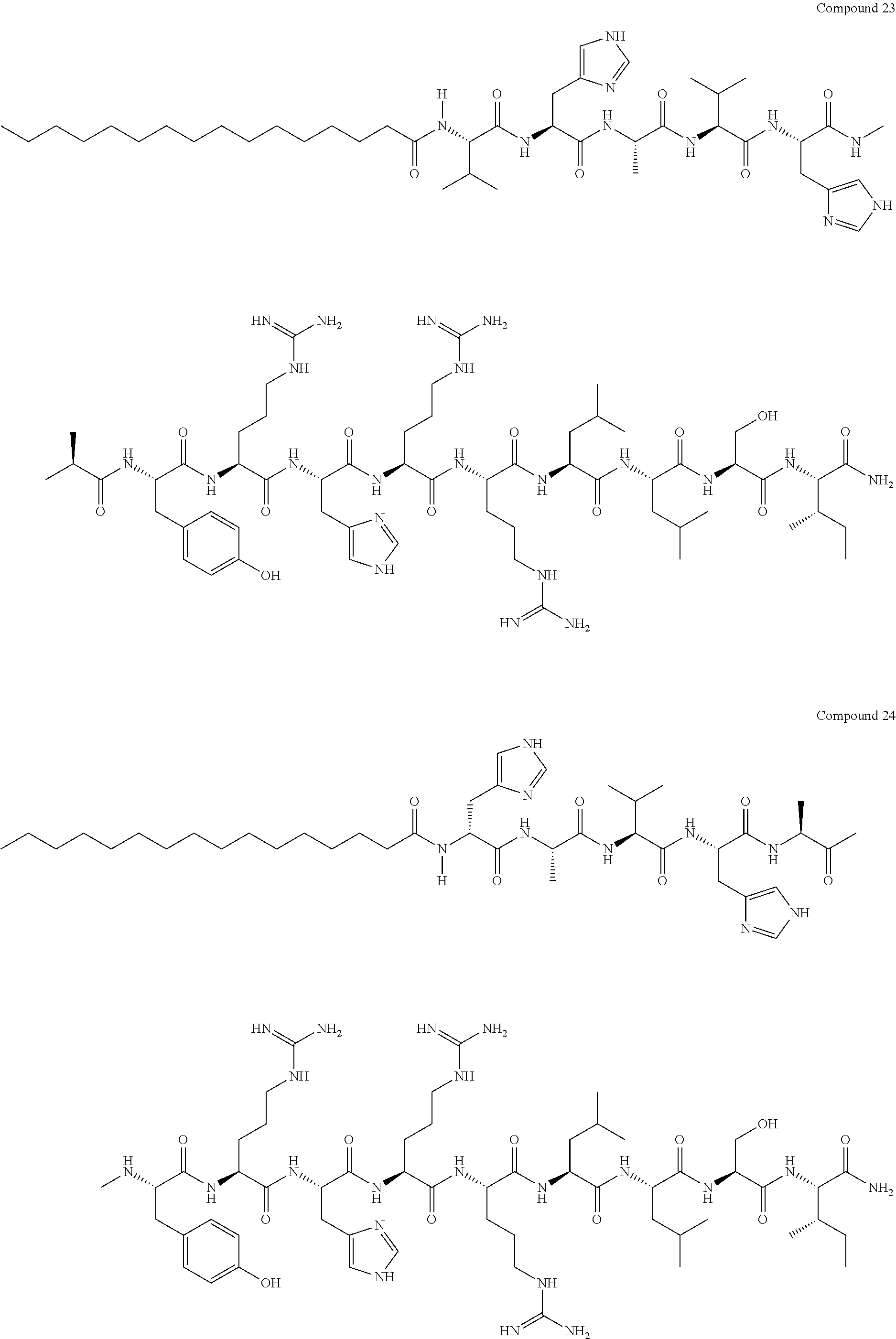 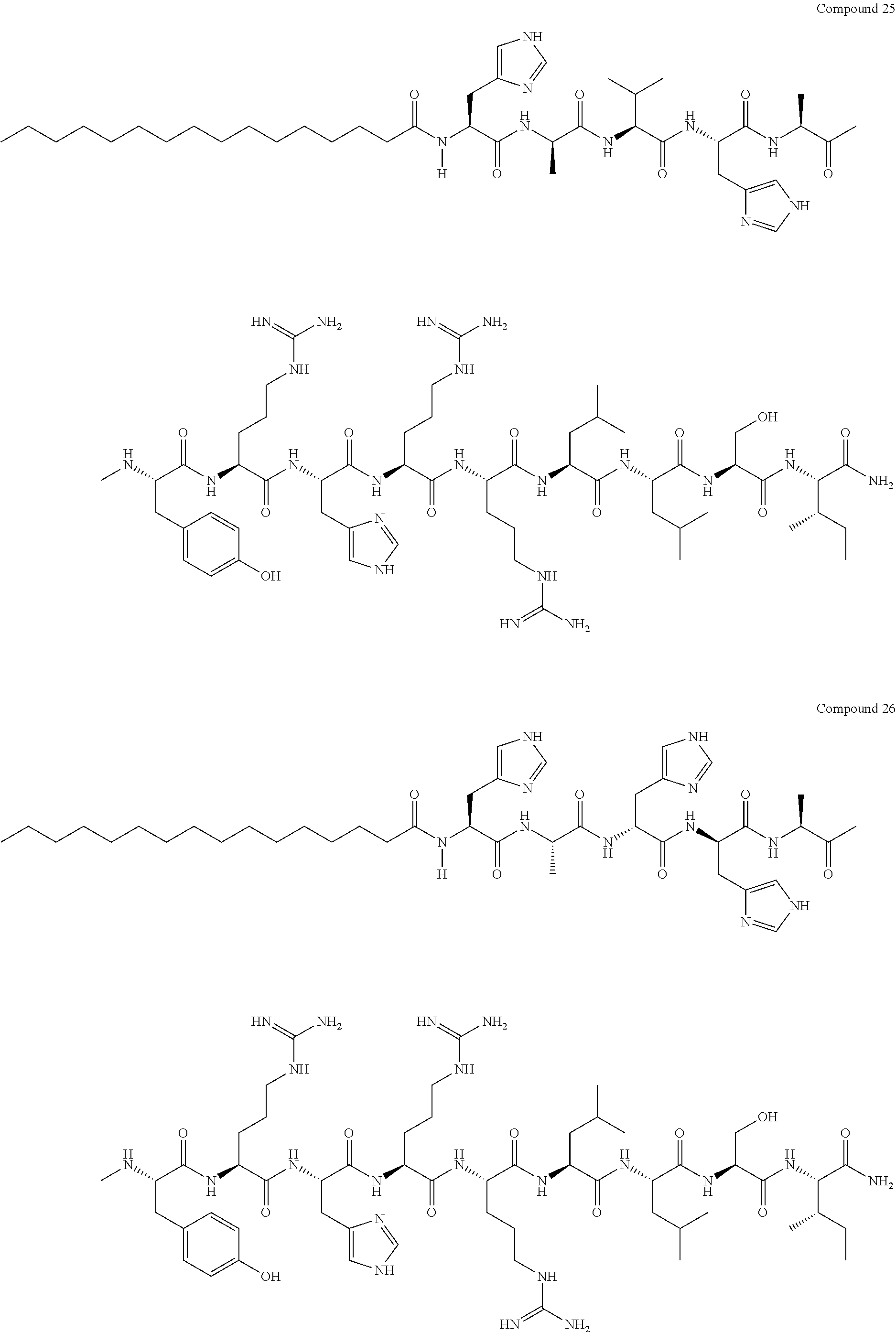 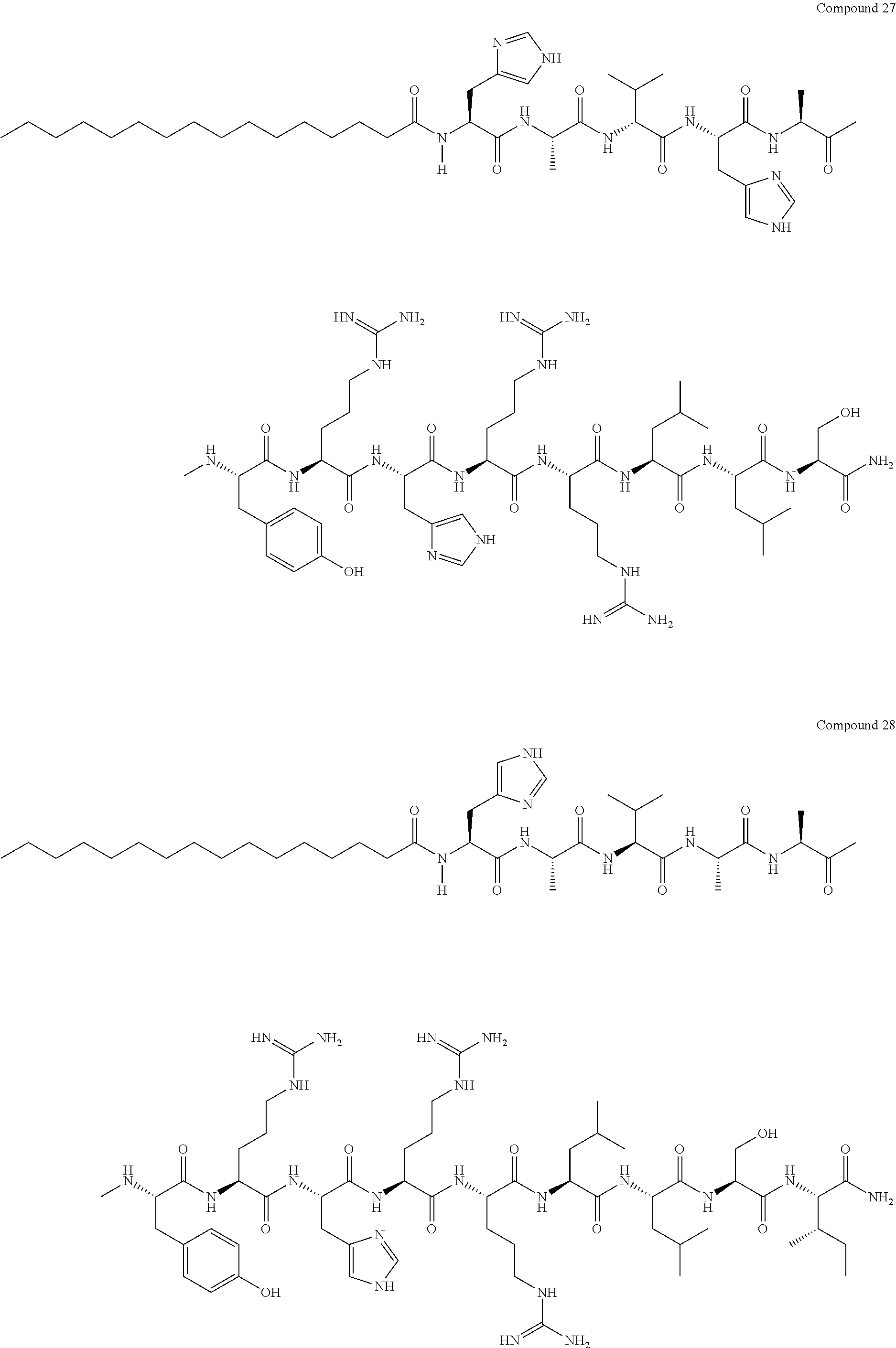 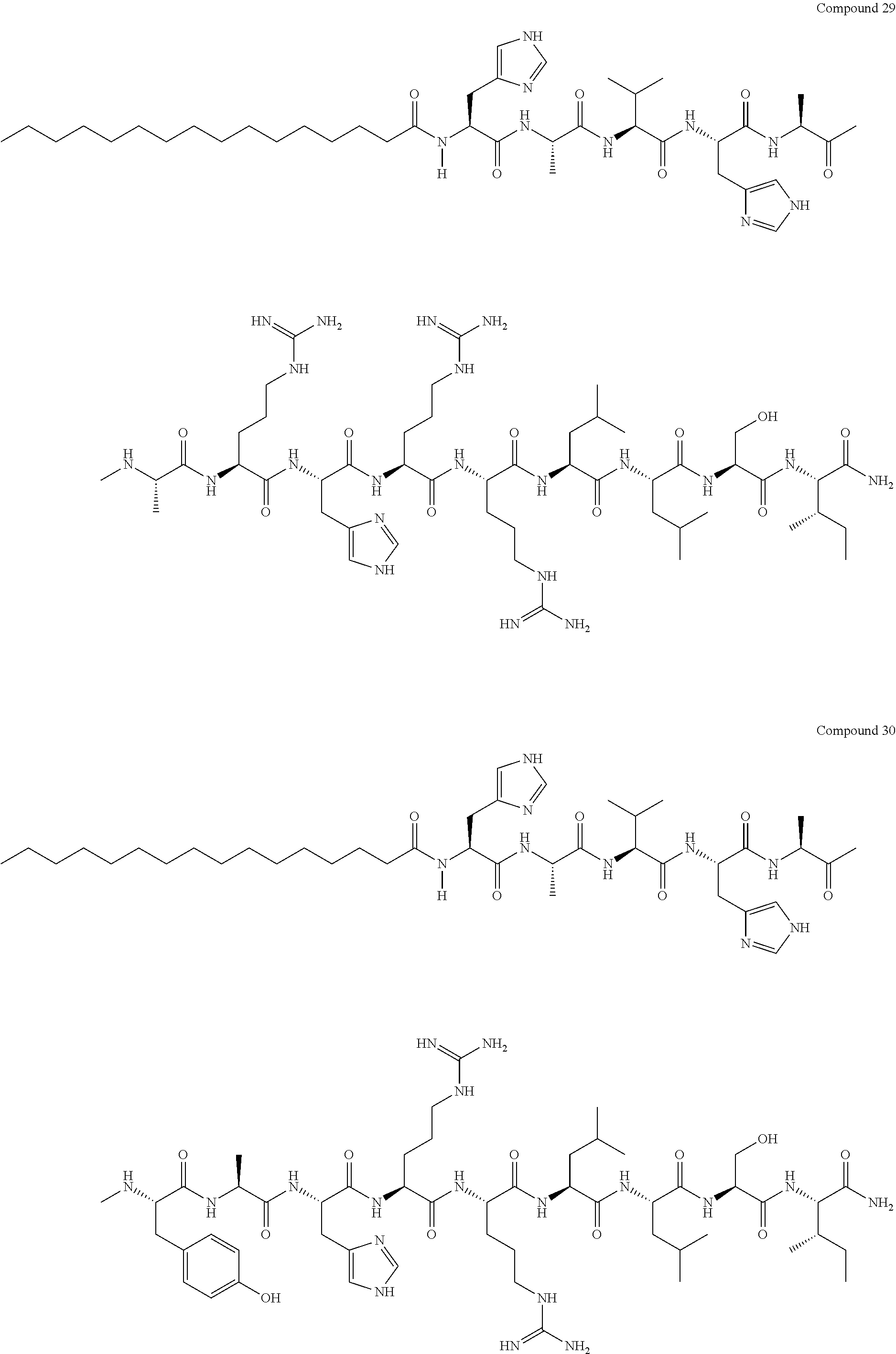 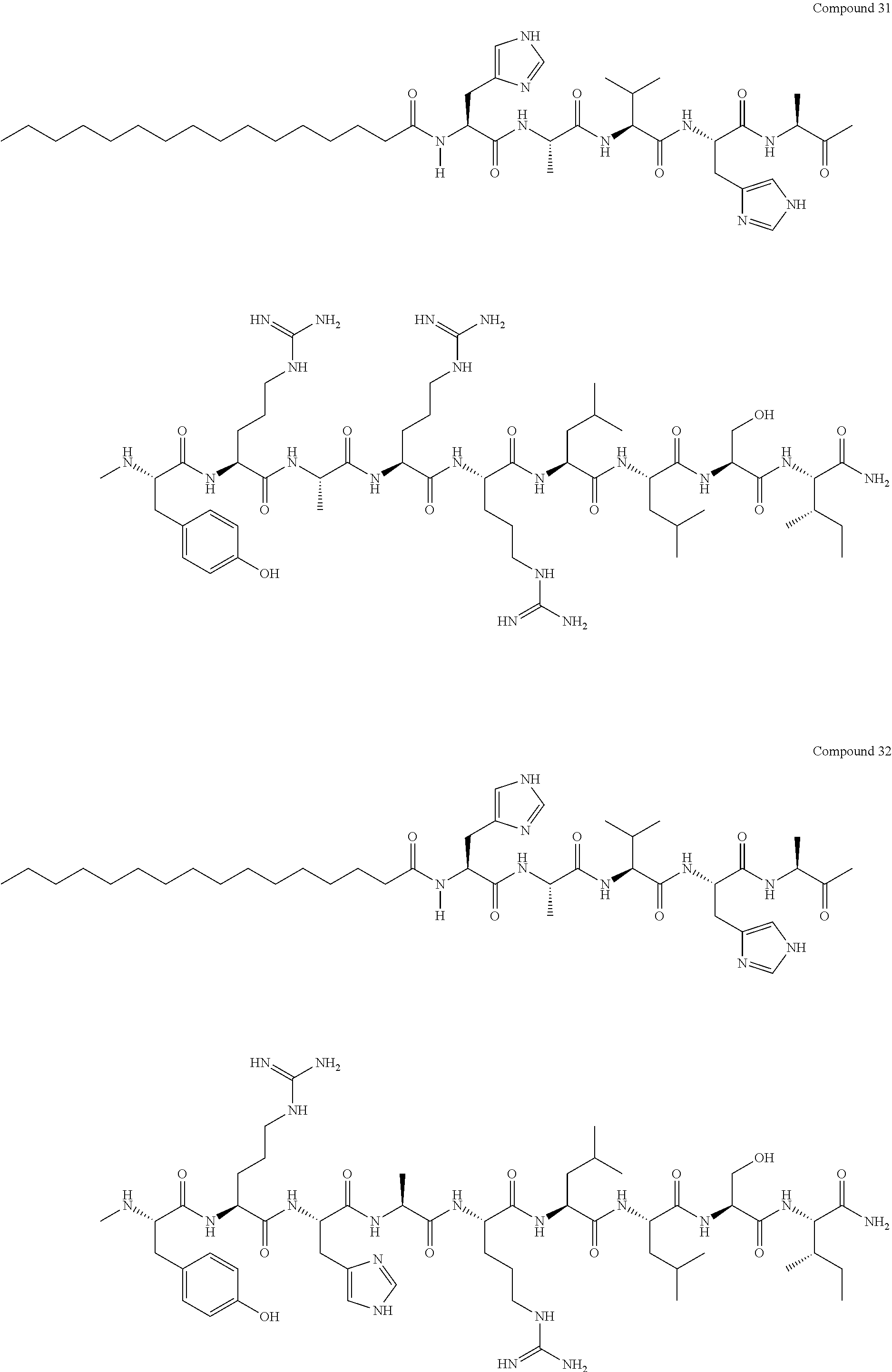 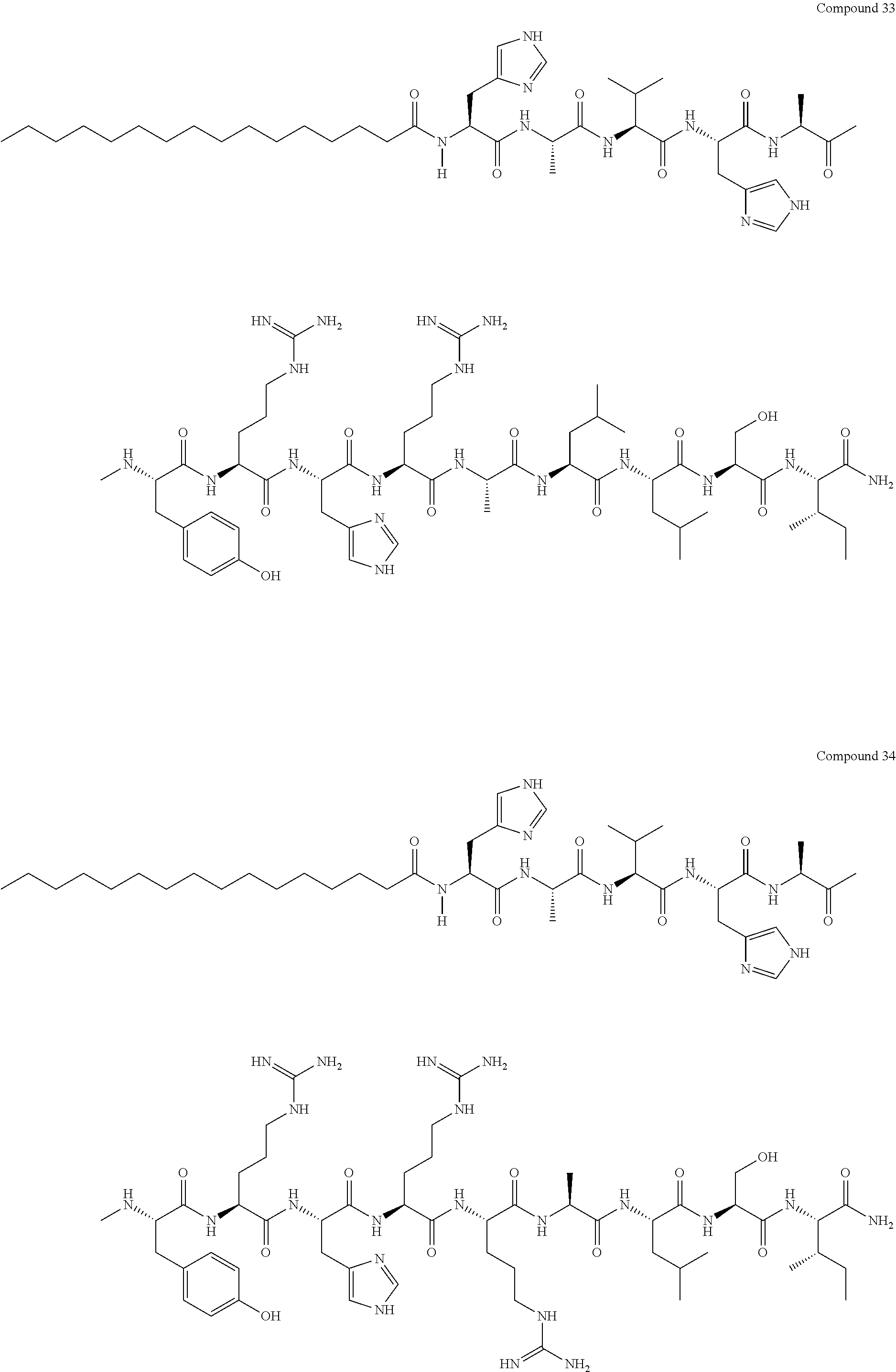 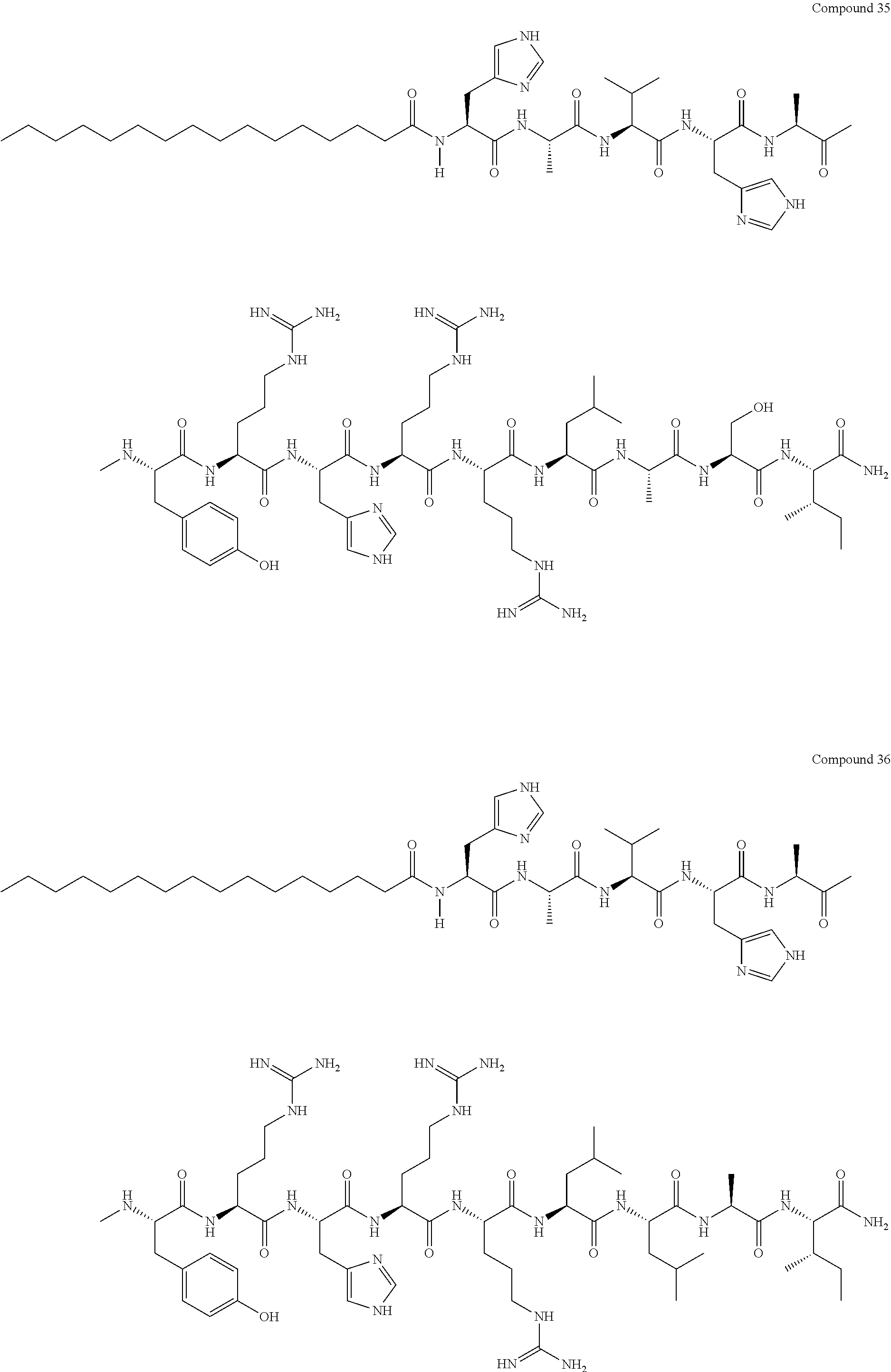 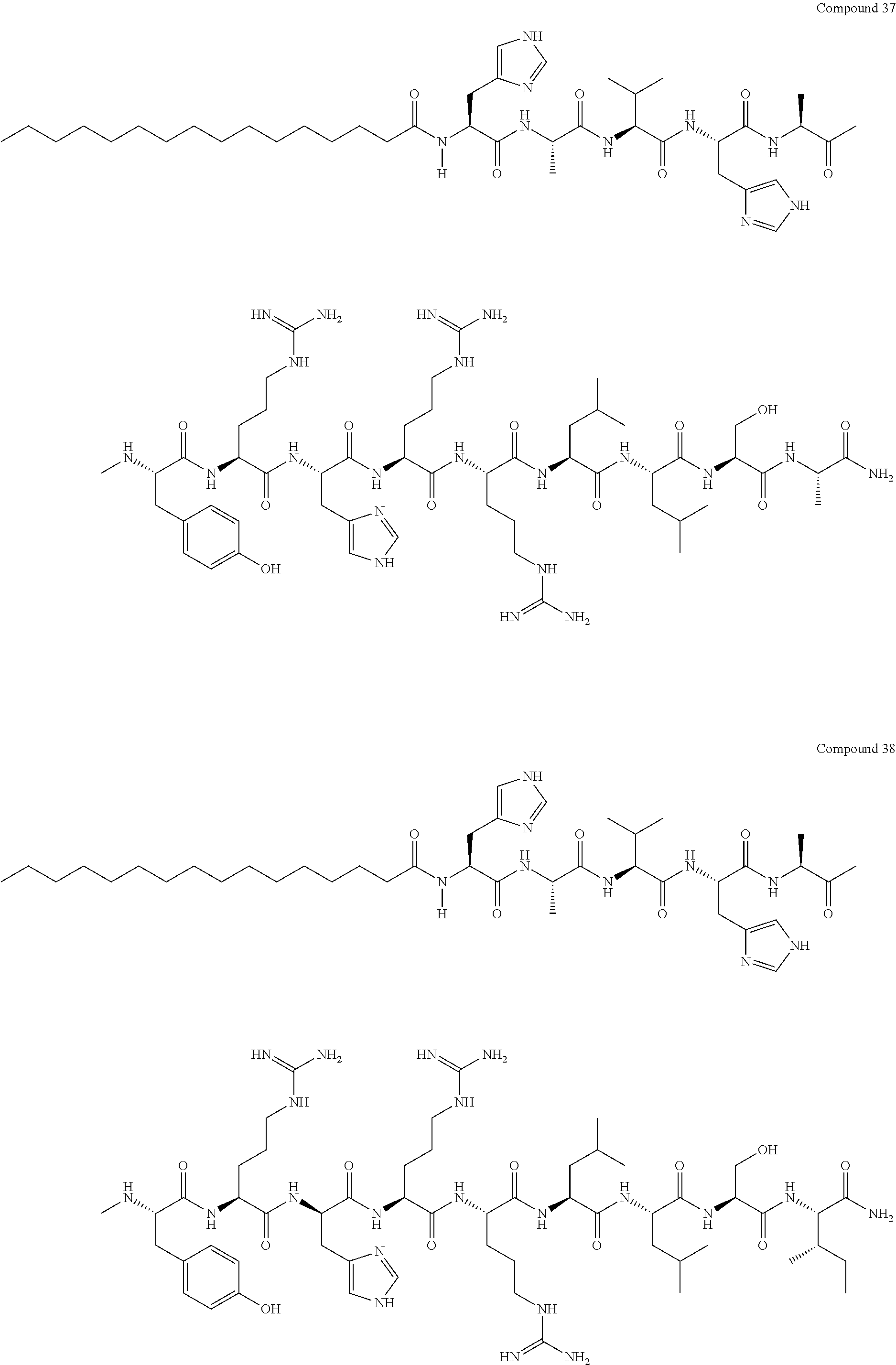 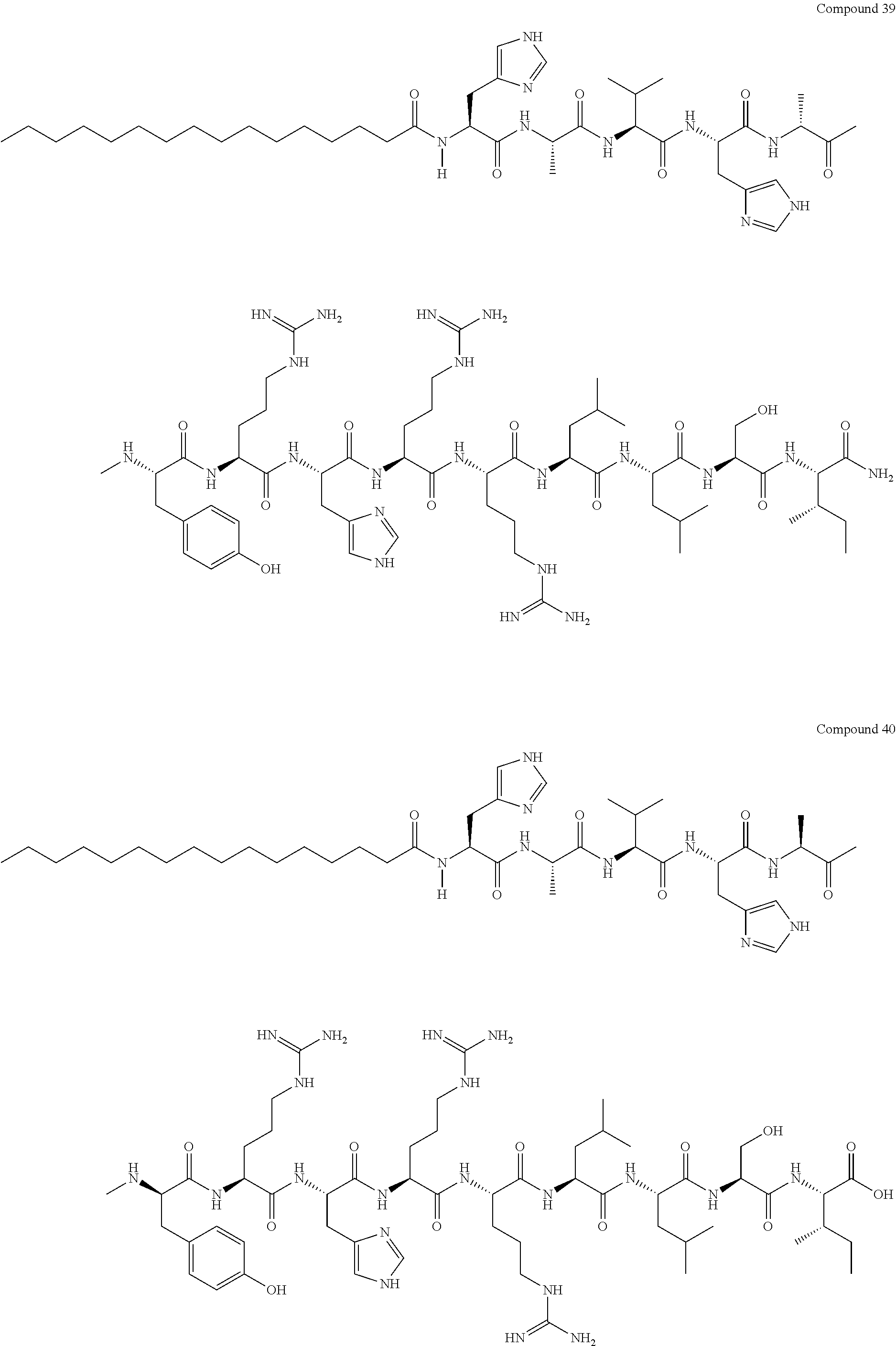 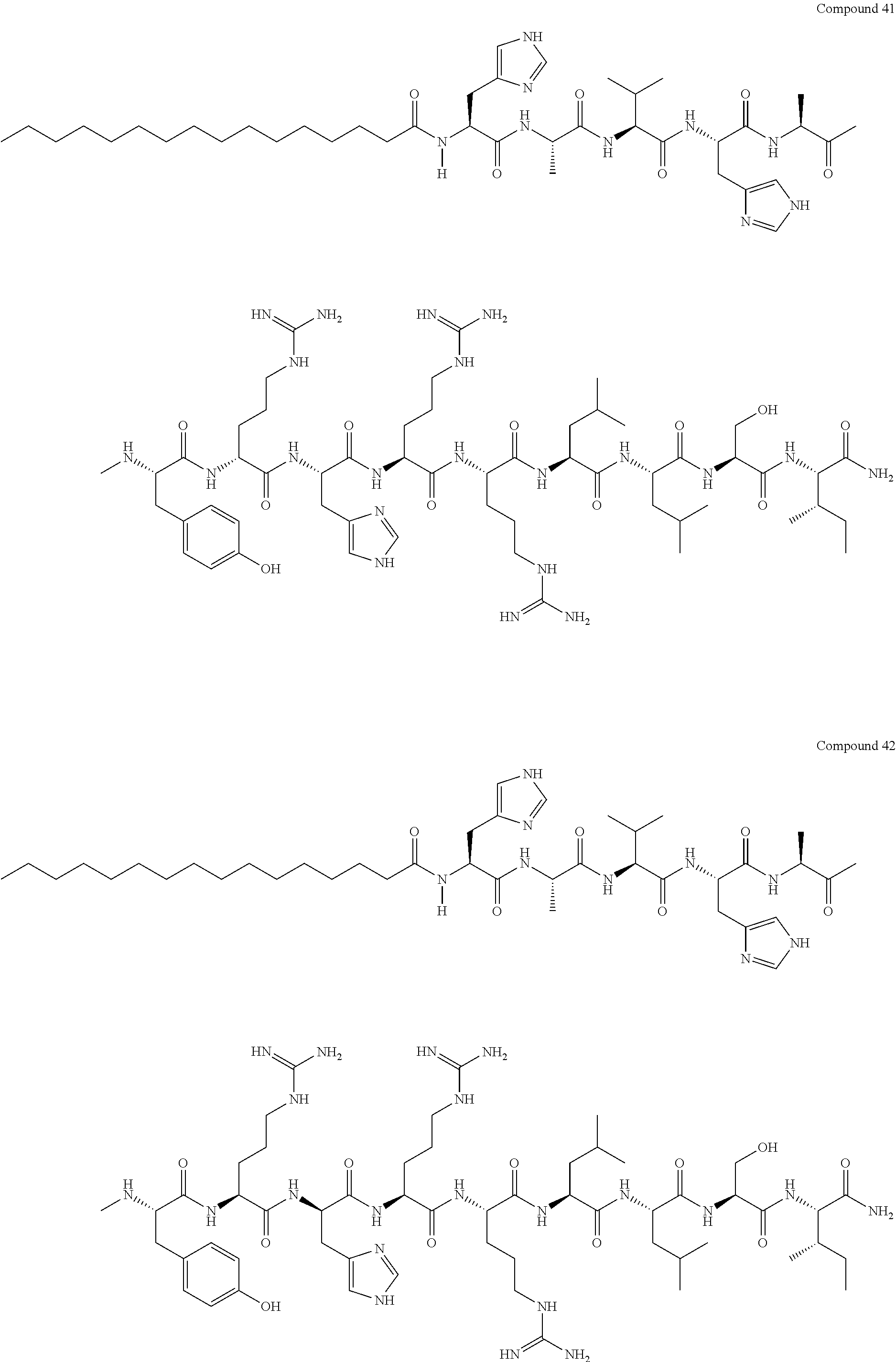 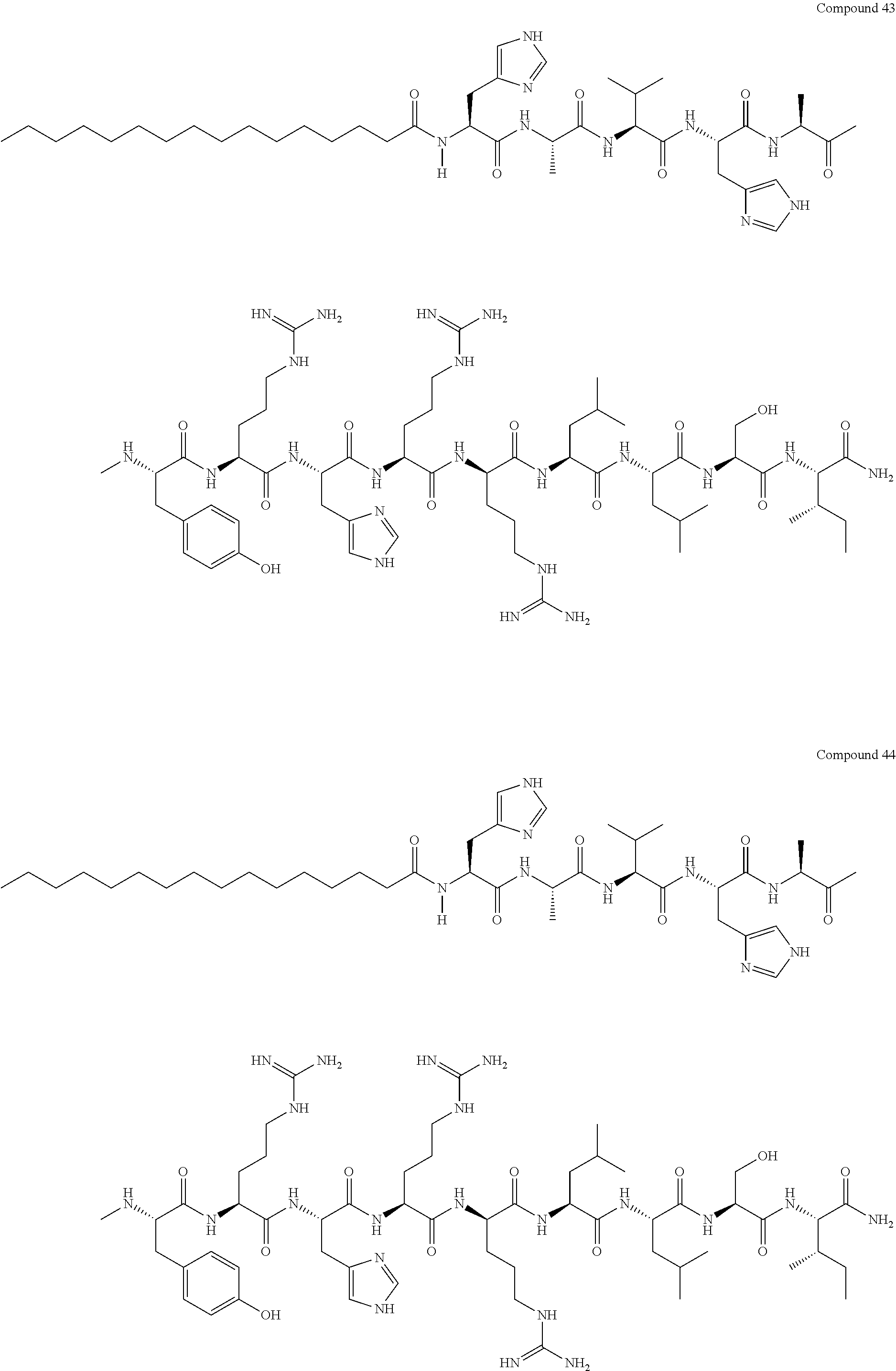 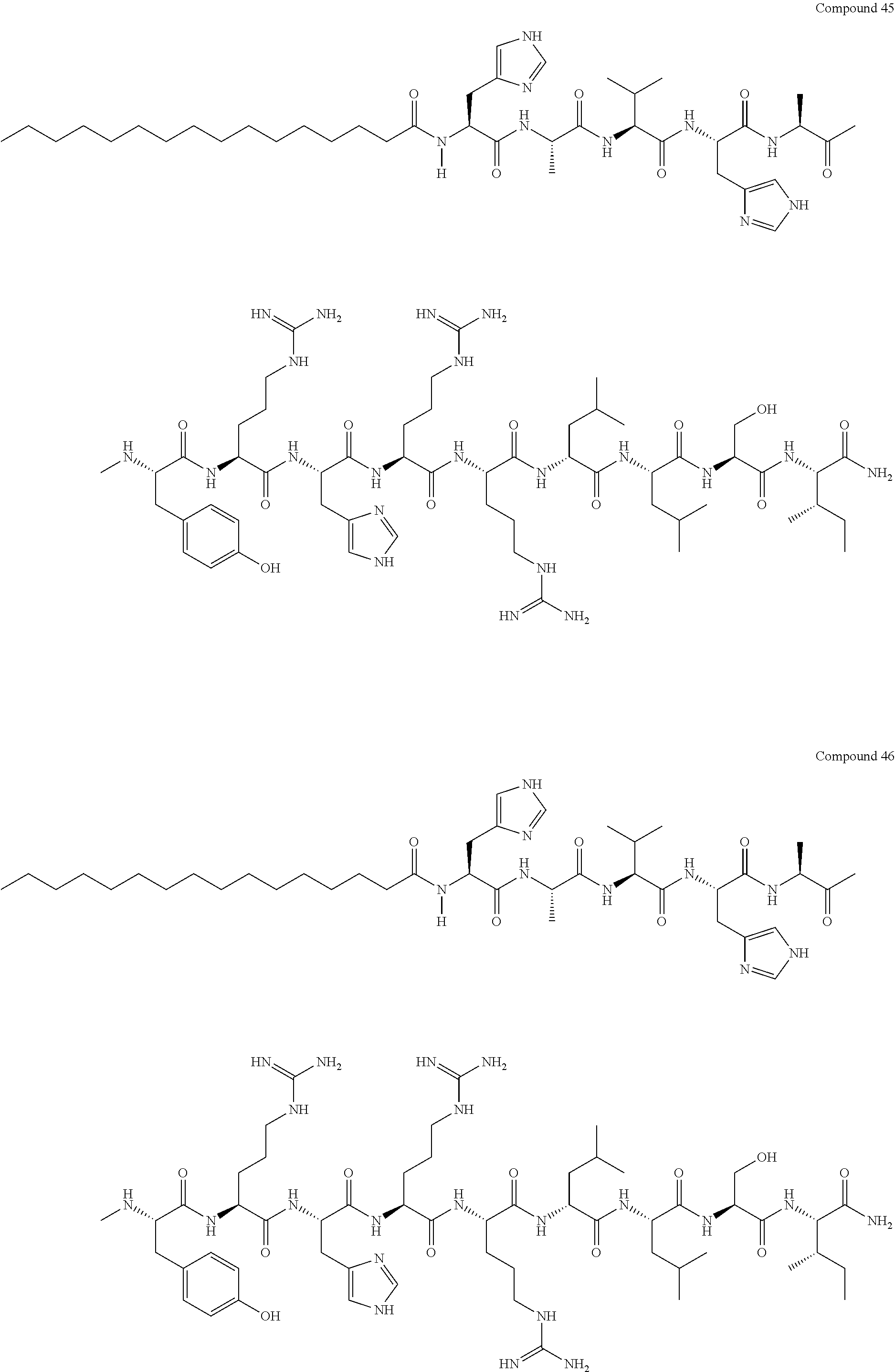 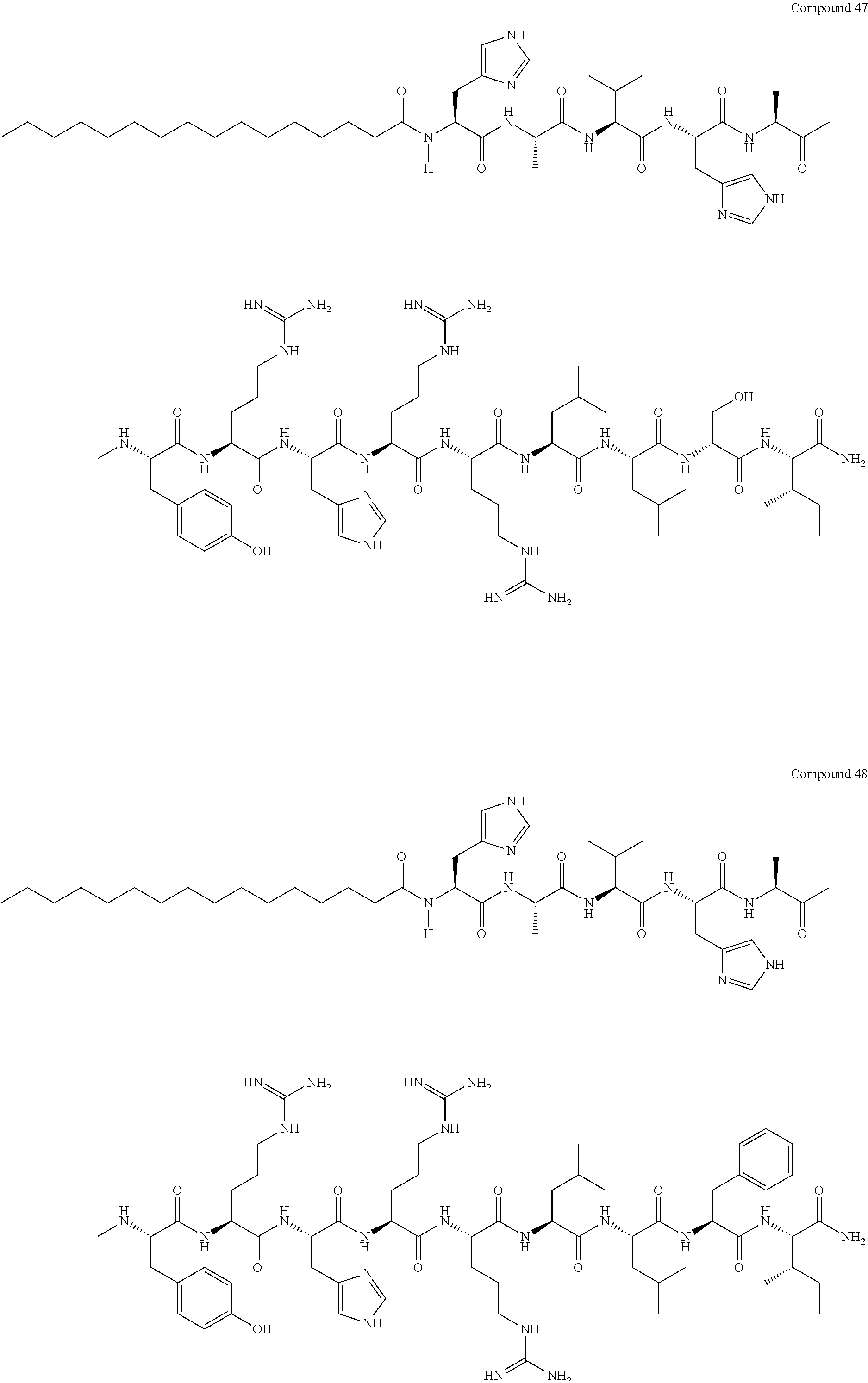 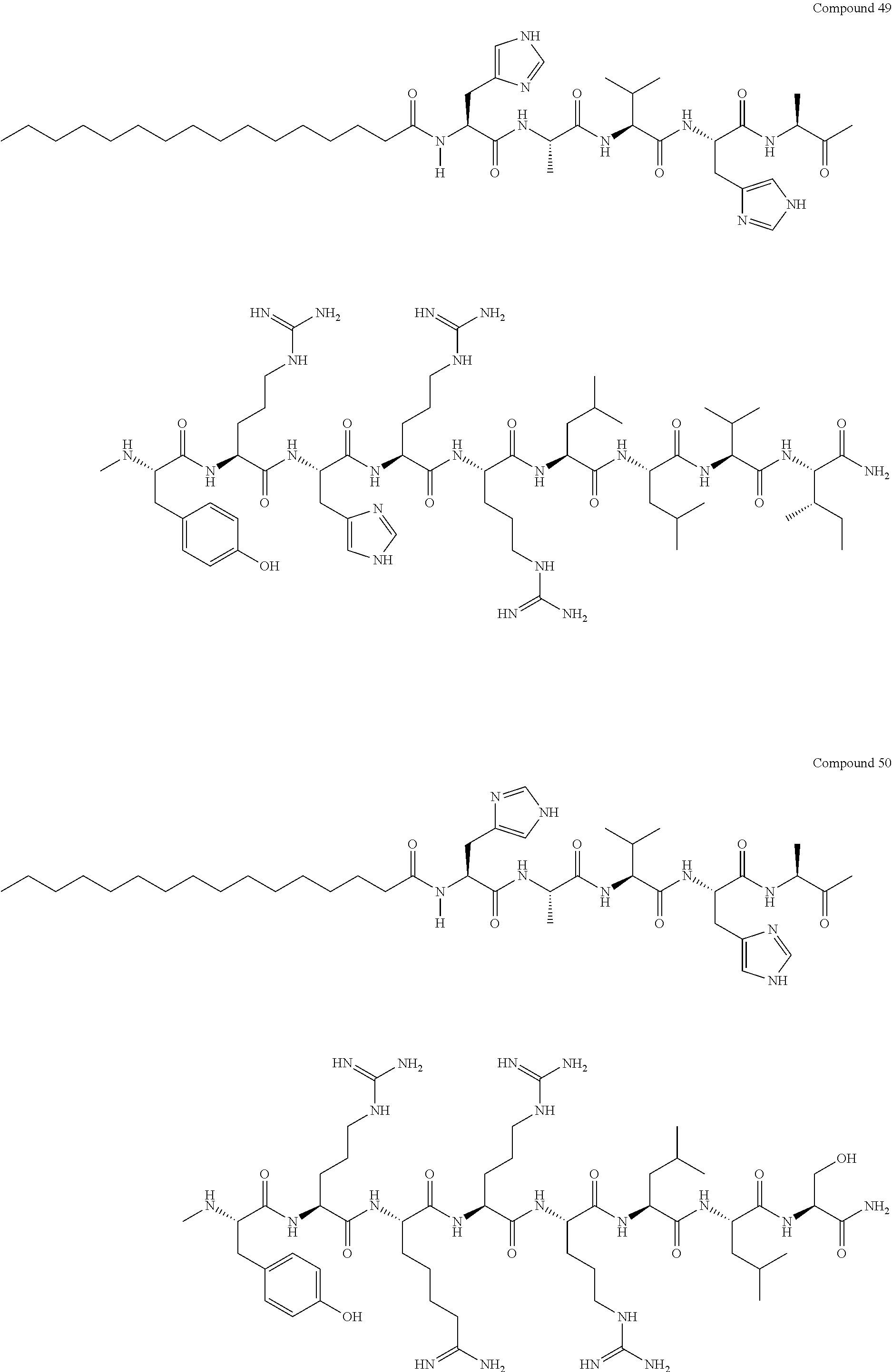 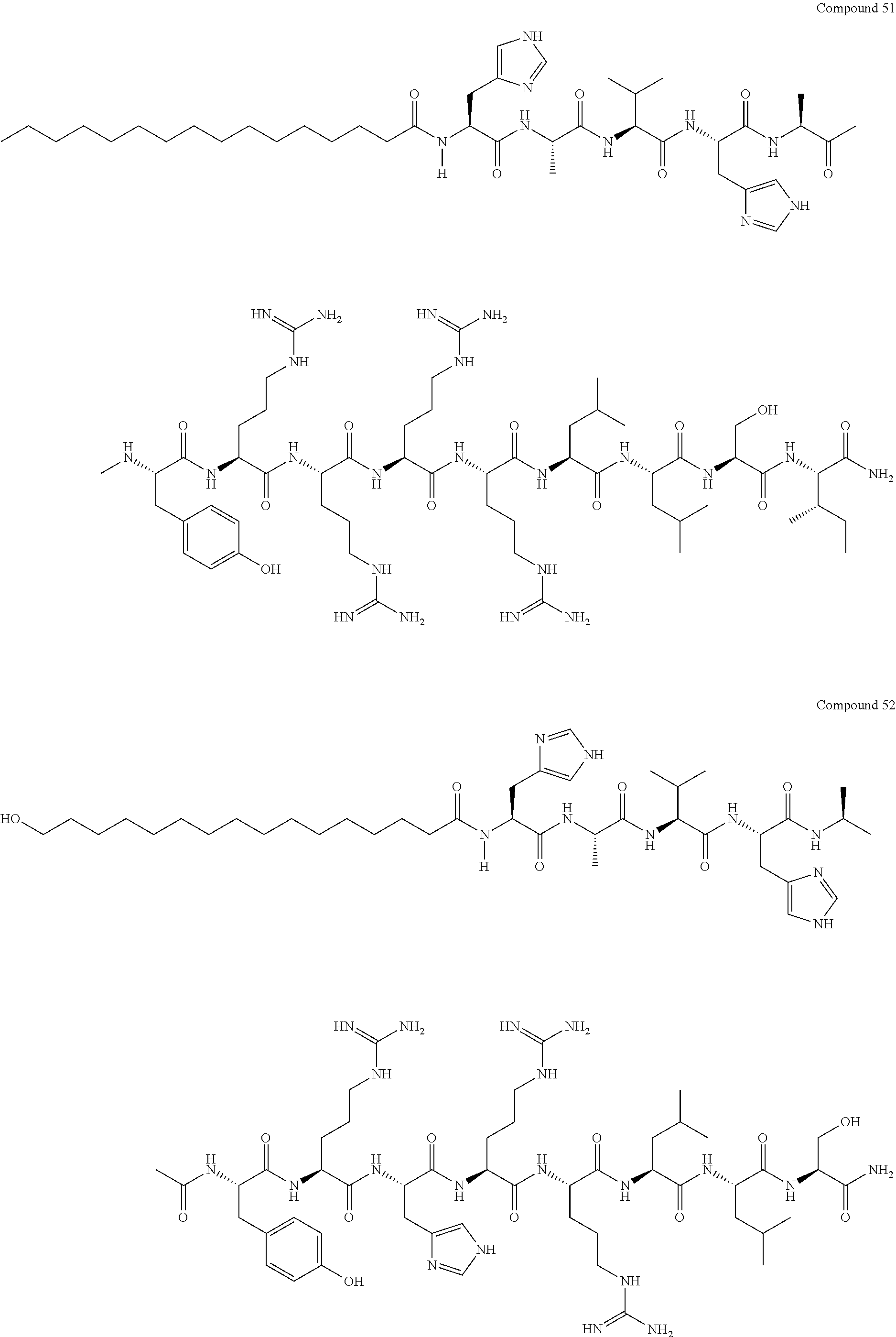 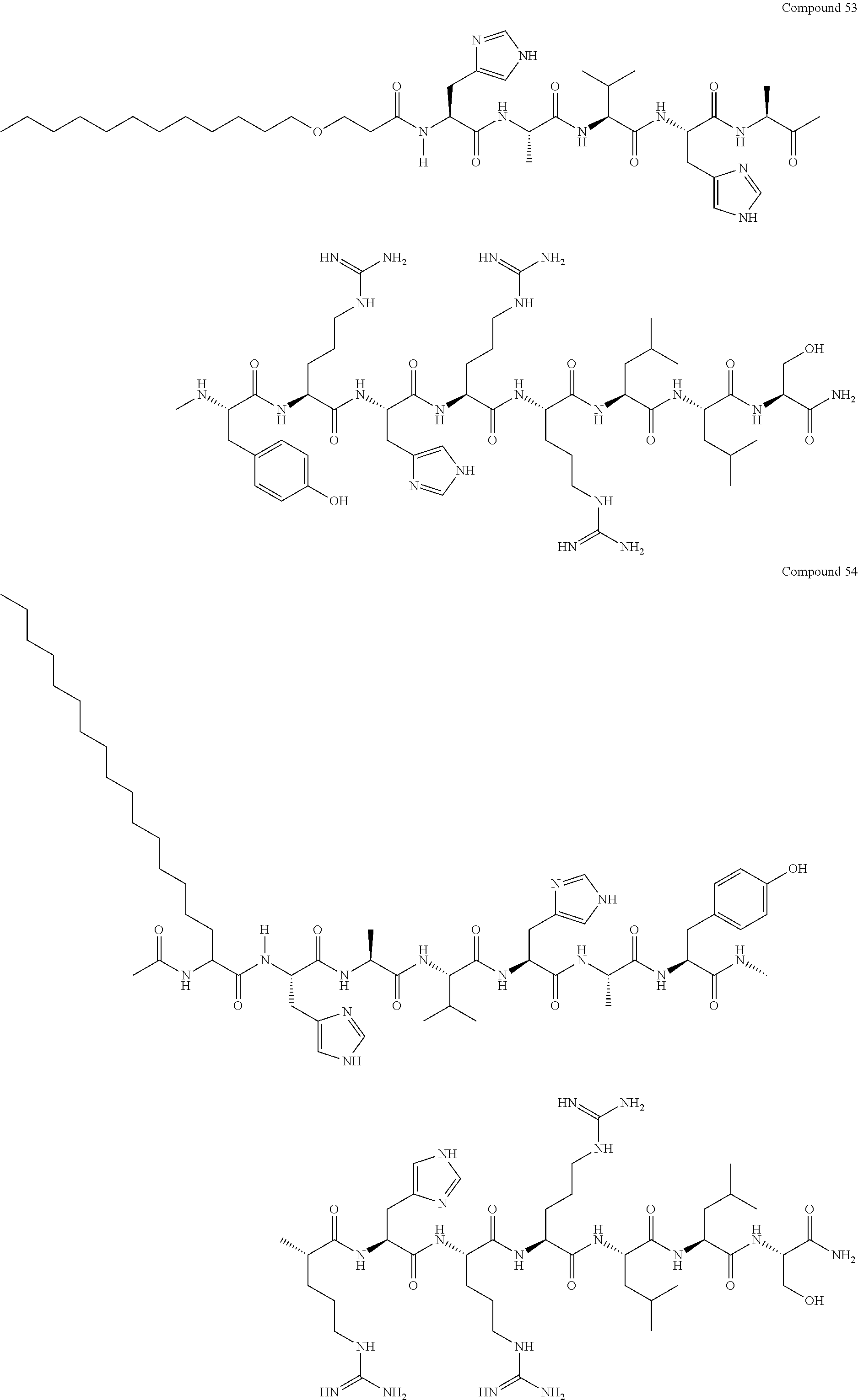 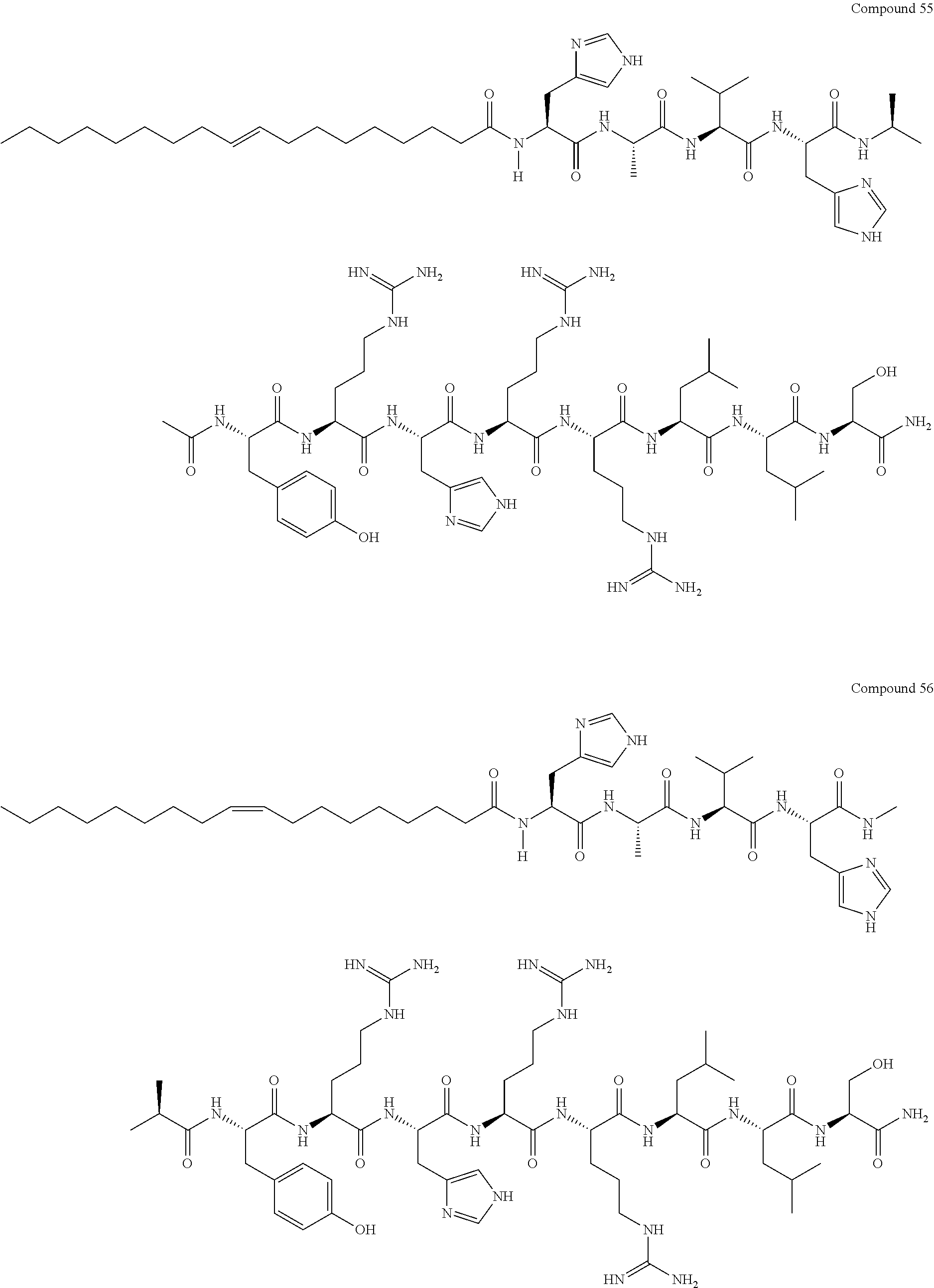
or a pharmaceutically acceptable salt thereof of any of the forgoing compounds.

15. A compound represented by Formula C or a pharmaceutically acceptable salt thereof, wherein:
T-L-W₁-W₂-W₃-W₄-W₅-W₆-W₇-W₈-W₉-W₁₀-W₁₁-W₁₂-W₁₃-W₁₄-W₁₅-
W₁₆-W₁₇-W₁₈-W₁₉-W₂₀-W₂₁-W₂₂-W₂₃-R₁;
wherein L is a linking moiety represented by C(O) and bonded to the N terminal nitrogen of W₁ or the next present amino acid residue if W1 is absent; T is a lipophilic tether moiety bonded to L; and R₁ is OR₂ or N(R₂)₂, wherein each R₂ is independently H or alkyl, wherein at least three contiguous W₁-W₂₃ amino acid residues are present and wherein:
W₁ is a glycine residue, a histidine residue or absent,
W₂ is a valine, a phenylalanine residue, a glycine residue or absent,
W₃ is a valine residue, an arginine residue, a serine residue or absent,
W₄ is a histidine residue, a lysine residue, a glycine residue or absent,
W₅ is an arginine residue, a glutamic residue acid or absent,
W₆ is a leucine residue, an arginine residue or absent,
W₇ is an arginine residue, an isoleucine residue or absent,
W₈ is a glutamine residue, a glutamic acid residue, an asparagine residue, a threonine residue or absent,
W₉ is an alanine residue, a glycine residue or absent,
W₁₀ is a glutamine residue, a leucine residue, an asparagine residue, a threonine residue or absent,
W₁₁ is an arginine residue or absent,
W₁₂ is an arginine residue or lysine,
W₁₃ is a proline residue or arginine,
W₁₄ is a glutamine residue, an arginine residue, an asparagine residue or a threonine residue,
W₁₅ is an arginine residue,
W₁₆ is a glutamine residue, a leucine residue, an asparagine residue, a threonine residue or absent,
W₁₇ is a lysine residue or absent,
W₁₈ is an alanine residue or absent,
W₁₉ is a valine residue or absent,
W₂₀ is an arginine residue or absent,
W₂₁ is a valine residue or absent,
W₂₂ is an alanine residue or absent, and
W₂₃ is an isoleucine residue or absent.

16. The compound of claim 15, wherein:
W₁ is a glycine residue or absent,
W₂ is a valine residue or absent,
W₃ is a valine residue or absent,
W₄ is a histidine residue or absent,
W₅ is an arginine residue or absent,
W₆ is a leucine residue or absent,
W₇ is an arginine residue, or absent,
W₈ is a glutamine residue, or absent,
W₉ is an alanine residue or absent,
W₁₀ is a glutamine residue or absent,
W₁₁ is an arginine residue or absent,
W₁₂ is an arginine residue,
W₁₃ is a proline residue,
W₁₄ is a glutamine residue,
W₁₅ is an arginine residue,
W₁₆ is a glutamine residue, or absent,
W₁₇ is a lysine residue or absent,
W₁₈ is an alanine residue or absent,
W₁₉ is a valine residue or absent,
W₂₀ is an arginine residue or absent,
W₂₁ is a valine residue or absent,
W₂₂ is an alanine residue or absent, and
W₂₃ is an isoleucine residue or absent.

17. The compound of claim 15, wherein W₁₈-W₂₃ are absent.

18. The compound of claim 15, wherein or W₁-W₄ are absent and W₅ is an arginine residue.

19. The compound of claim 15, wherein the compound is selected from compounds 57-79 or a pharmaceutically acceptable salt thereof.

20. A compound represented by Formula D or a pharmaceutically acceptable salt thereof:
T-L-Z₁-Z₂-Z₃-Z₄-Z₅-Z₆-Z₇-Z₈-Z₉-Z₁₀-Z₁₁-Z₁₂-Z₁₃-Z₁₄-Z₁₅-
Z₁₆-Z₁₇-Z₁₈-Z₁₉-Z₂₀-Z₂₁-Z₂₂-Z₂₃-Z₂₃-Z₂₄-Z₂₅-Z₂₆-Z₂₇-Z₂₈-
Z₂₉Z₃₀-Z₃₁Z₃₂-Z₃₃-Z₃₄-Z₃₅-Z₃₆-Z₃₇-Z₃₈-Z₃₉-Z₄₀-Z₄₁-Z₄₂-Z₄₃-
Z₄₄-Z₄₅-Z₄₆-Z₄₇-Z₄₈-R₁;
wherein L is a linking moiety represented by C(O) and bonded to the N terminal nitrogen of Z₁ or the next present amino acid if Z₁ is absent; T is a lipophilic tether moiety bonded to L; and R₁ is OR₂ or N(R₂)₂, wherein each R₂ is independently H or alkyl, wherein at least three contiguous Z₁-Z₂₃ amino acid residues are present and wherein:
Z₁ is an alanine residue or absent,
Z₂ is a glycine residue or absent,
Z₃ is a valine residue, or absent,
Z₄ is a lysine residue or absent,
Z₅ is a phenylalanine residue or absent,
Z₆ is an arginine residue or absent,
Z₇ is a serine residue or absent,
Z₈ is an aspartic acid residue or absent,
Z₉ is a leucine residue or absent,
Z₁₀ is a serine residue or absent,
Z₁₁ is an arginine residue or absent,
Z₁₂ is a leucine residue,
Z₁₃ is a leucine residue or arginine,
Z₁₄ is a threonine residue,
Z₁₅ is a lysine residue,
Z₁₆ is a leucine residue or absent,
Z₁₇ is a glycine residue or absent,
Z₁₈ is a cysteine residue, a serine residue or absent,
Z₁₉ is a threonine residue or absent,
Z₂₀ is a glycine or absent residue or absent,
Z₂₁ is a proline residue or absent,
Z₂₂ is an alanine residue or absent,
Z₂₃ is a serine residue or absent,
Z₂₄ is a leucine residue or absent,
Z₂₅ is a cysteine residue, a serine residue or absent,
Z₂₆ is a glutamine residue or absent,
Z₂₇ is a leucine residue or absent,
Z₂₈ is a phenylalanine residue or absent,
Z₂₉ is a proline residue or absent,
Z₃₀ is a serine residue or absent,
Z₃₁ is a tryptophan residue or absent,
Z₃₂ is an arginine residue or absent,
Z₃₃ is an arginine residue or absent,
Z₃₄ is a serine residue or absent,
Z₃₅ is a serine residue or absent,
Z₃₆ is a leucine residue or absent,
Z₃₇ is a serine residue or absent,
Z₃₈ is a glutamic residue acid or absent,
Z₃₉ is a serine residue or absent,
Z₄₀ is a glutamic residue acid or absent,
Z₄₁ is an asparagine residue or absent,
Z₄₂ is a alanine residue or absent,
Z₄₃ is a threonine residue or absent,
Z₄₄ is a serine residue or absent,
Z₄₅ is a leucine residue or absent,
Z₄₆ is a threonine residue or absent,
Z₄₇ is a threonine residue or absent, and
Z₄₈ is a phenylalanine residue or absent.

21. The compound of claim 20, wherein Z₂₆-Z₄₈ is absent or Z₁₈-Z₄₈ is absent or Z₁₆-Z₄₈ is absent.

22. The compound of claim 20, wherein Z₁-Z₁₁ is absent Z₁-Z₈ is absent or Z₁-Z₅ is absent.

23. The compound of claim 20, wherein the compound is selected from compounds 80-83 or a pharmaceutically acceptable salt thereof.

24. A compound represented by Formula I:
T-L-P,
or a pharmaceutically acceptable salt thereof, wherein:
P is a peptide sequence selected from: SEQ ID NOS: 1-93;
L is a linking moiety represented by C(O) and bonded to P at an N terminal nitrogen of an N-terminal amino-acid residue;
and T is a lipophilic tether moiety bonded to L.

25. The compound of claim 24, wherein P is selected from SEQ ID NOS: 1-13.

26. The compound of claim 24, wherein P is selected from SEQ ID NOS: 14-60.

27. The compound of claim 24, wherein P is selected from SEQ ID NOS: 61-87.

28. The compound of claim 24, wherein P is selected from SEQ ID NOS: 88-93.

29. The compound of claim 1, wherein T is an optionally substituted (C₆-C₃₀)alkyl, (C₆-C₃₀)alkenyl, (C₆-C₃₀)alkynyl, wherein 0-3 carbon atoms are replaced with oxygen, sulfur, nitrogen or a combination thereof.

30. The compound of claim 29, wherein T is selected from the group consisting of: CH₃(CH₂)₁₆, CH₃(CH₂)₁₅, CH₃(CH₂)₁₄, CH₃(CH₂)₁₃ , CH₃(CH₂)₁₂, CH₃(CH₂)₁₁, CH₃(CH₂)₁₀ , CH₃(CH₂)₉, CH₃(CH₂)₈, CH₃(CH₂)₉OPh-, CH₃(CH₂)₆C═C(CH₂)₆, CH₃(CH₂)₁₁O(CH₂)₃, and CH₃(CH₂)₉O(CH₂)₂.

31. The compound of claim 1, wherein T is a fatty acid derivative.

32. The compound of claim 31, wherein the fatty acid is selected from the group consisting of: butyric acid, caproic acid, caprylic acid, capric acid, lauric acid, myristic acid, palmitic acid, stearic acid, arachidic acid, behenic acid, lignoceric acid, myristoleic acid, palmitoleic acid, oleic acid, linoleic acid, α-linolenic acid, arachidonic acid, eicosapentaenoic acid, erucic acid, docosahexaenoic acid.

33. The compound of claim 1, wherein T is a bile acid derivative.

34. The compound of claim 33, wherein the bile acid is selected from the group consisting of: lithocholic acid, chenodeoxycholic acid, deoxycholic acid, cholanic acid, cholic acid, ursocholic acid, ursodeoxycholic acid, isoursodeoxycholic acid, lagodeoxycholic acid, dehydrocholic acid, hyocholic acid, and hyodeoxycholic acid.

35. The compound of claim 1, wherein T is selected from sterols; progestagens; glucocorticoids; mineralcorticoids; androgens; and estrogens.

36. The compound of claim 1, wherein TL is selected from:
CH₃(CH₂)₁₅—C(O);
CH₃(CH₂)₁₃—C(O);
CH₃(CH₂)₉O(CH₂)₂C(O);
CH₃(CH₂)₁₀O(CH₂)₂C(O);
CH₃(CH₂)₆C═C(CH₂)₆—C(O);
LCA-C(O); and
CH₃(CH₂)₉OPh-C(O) wherein

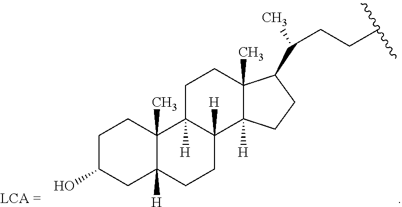

37. The compound of claim 1, wherein T is selected from:

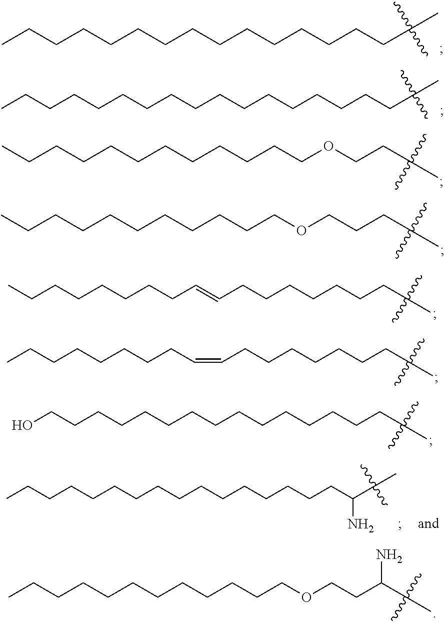

38. A method of treating diseases and conditions associated with CXCR5 receptor modulation in a patient in need thereof comprising administering to said patient and effective amount of a compound of claim 1.

39. The method of claim 38, wherein the disease or condition is selected from: autoimmune disease, Primary Sjögren's Syndrome, chronic lymphocytic leukemia, Burkitt Lymphoma, colon and breast cancer tumor metastasis, Multiple Sclerosis and compromised immune function.

40. The method of claim 39, wherein the autoimmune disease is selected from:
lupus, HIV and rheumatoid arthritis.

41. A pharmaceutical composition comprising a compound of claim 1, and a pharmaceutically acceptable carrier.
